SEQUENCE LISTING (76 sequences)

SEQ ID NO: 1 - protein, 10 aa, synthetic construct
WSDGLALNAL

SEQ ID NO: 2 - protein, 20 aa, synthetic construct
FDWNSVVCQQSATQRLEHAF

SEQ ID NO: 3 - protein, 9 aa, synthetic construct
WSDGLALNA

SEQ ID NO: 4 - protein, 9 aa, synthetic construct
WSNGLGLDA

SEQ ID NO: 5 - protein, 9 aa, synthetic construct
WSDGTGLNA

SEQ ID NO: 6 - protein, 9 aa, synthetic construct
LSDGTGLNA

SEQ ID NO: 7 - protein, 10 aa, synthetic construct
WSNGLGLDAL

SEQ ID NO: 8 - protein, 10 aa, synthetic construct
WSDGTGLNAL

SEQ ID NO: 9 - protein, 10 aa, synthetic construct
LSDGTGLNAL

SEQ ID NO: 10 - protein, 28 aa, synthetic construct
RPDLFDWNSVVSQQSATQRLEHAFNIAR

SEQ ID NO: 11 - protein, 28 aa, synthetic construct
RPDLFDWNSVVSQKSAKYRLEHAFNIAR

SEQ ID NO: 12 - protein, 28 aa, synthetic construct
RPDLFDWNSVVQQDSAKYRLQHAFNIAR

SEQ ID NO: 13 - protein, 28 aa, synthetic construct
RPDLFDWNSVVKQDSAKYRLKHAFNIAR

SEQ ID NO: 14 - protein, 28 aa, synthetic construct
RPDLFDWDSVVQQQSAKYRLQHAFNIAR

SEQ ID NO: 15 - protein, 28 aa, synthetic construct
RPDLFDWNSVVNQQSAQYRLKHAFNIAR

SEQ ID NO: 16 - protein, 20 aa, synthetic construct
FDWNSVVCQKSAKYRLEHAF

SEQ ID NO: 17 - protein, 20 aa, synthetic construct
FDWNSVVQQDSAKYRLQHAF

SEQ ID NO: 18 - protein, 20 aa, synthetic construct
FDWNSVVKQDSAKYRLKHAF

SEQ ID NO: 19 - protein, 20 aa, synthetic construct
FDWDSVVQQQSAKYRLQHAF

SEQ ID NO: 20 - protein, 20 aa, synthetic construct
FDWNSVVNQQSAQYRLKHAF

SEQ ID NO: 21 - DNA, 209 bp, synthetic construct
  source 1..209: mol_type = other DNA
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggtttttttt

SEQ ID NO: 22 - DNA, 752 bp, synthetic construct
  source 1..752: mol_type = other DNA
gctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaag

SEQ ID NO: 23 - DNA, 255 bp, synthetic construct
  source 1..255: mol_type = other DNA
tgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagaca

SEQ ID NO: 24 - DNA, 327 bp, synthetic construct
  source 1..327: mol_type = other DNA
agtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtag

SEQ ID NO: 25 - DNA, 333 bp, synthetic construct
  source 1..333: mol_type = other DNA
ctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacag

SEQ ID NO: 26 - DNA, 333 bp, synthetic construct
  source 1..333: mol_type = other DNA
aagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttg

SEQ ID NO: 27 - DNA, 147 bp, synthetic construct
  source 1..147: mol_type = other DNA
aaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctc

SEQ ID NO: 28 - DNA, 333 bp, synthetic construct
  source 1..333: mol_type = other DNA
cccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgc

SEQ ID NO: 29 - DNA, 327 bp, synthetic construct
  source 1..327: mol_type = other DNA
taagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggcca

SEQ ID NO: 30 - DNA, 327 bp, synthetic construct
  source 1..327: mol_type = other DNA
gagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctgg

SEQ ID NO: 31 - DNA, 327 bp, synthetic construct
  source 1..327: mol_type = other DNA
ttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtct

SEQ ID NO: 32 - DNA, 327 bp, synthetic construct
  source 1..327: mol_type = other DNA
atgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctga

SEQ ID NO: 33 - DNA, 312 bp, synthetic construct
  source 1..312: mol_type = other DNA
atgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctg

SEQ ID NO: 34 - DNA, 276 bp, synthetic construct
  source 1..276: mol_type = other DNA
taaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaat

SEQ ID NO: 35 - DNA, 327 bp, synthetic construct
  source 1..327: mol_type = other DNA
tacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaa

SEQ ID NO: 36 - DNA, 324 bp, synthetic construct
  source 1..324: mol_type = other DNA
cagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcag

SEQ ID NO: 37 - DNA, 212 bp, synthetic construct
  source 1..212: mol_type = other DNA
aagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagca

SEQ ID NO: 38 - DNA, 382 bp, synthetic construct
  source 1..382: mol_type = other DNA
gcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggt

SEQ ID NO: 39 - DNA, 294 bp, synthetic construct
  source 1..294: mol_type = other DNA
ctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttg

SEQ ID NO: 40 - DNA, 60 bp, synthetic construct
  source 1..60: mol_type = other DNA
ctcagtttcgaagactcaactttgcacaaattcacactgtccgtgaagaaacgatgatgg

SEQ ID NO: 41 - DNA, 327 bp, synthetic construct
  source 1..327: mol_type = other DNA
tgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtaca

SEQ ID NO: 42 - DNA, 321 bp, synthetic construct
  source 1..321: mol_type = other DNA
aggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgt

SEQ ID NO: 43 - DNA, 351 bp, synthetic construct
  source 1..351: mol_type = other DNA
cagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattg

SEQ ID NO: 44 - DNA, 303 bp, synthetic construct
  source 1..303: mol_type = other DNA
aagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttc

SEQ ID NO: 45 - DNA, 123 bp, synthetic construct
  source 1..123: mol_type = other DNA
aagagctgagggcaaagcagcctgacctagctcctggactgaccactattggagcctctcctactcagactgttactctggtgacacaacctgtggttactaaggaaactgccatctccaaac

SEQ ID NO: 46 - DNA, 330 bp, synthetic construct
  source 1..330: mol_type = other DNA
tagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttc

SEQ ID NO: 47 - DNA, 327 bp, synthetic construct
  source 1..327: mol_type = other DNA
agaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtga

SEQ ID NO: 48 - DNA, 348 bp, synthetic construct
  source 1..348: mol_type = other DNA
gtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctc

SEQ ID NO: 49 - DNA, 387 bp, synthetic construct
  source 1..387: mol_type = other DNA
tcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccg

SEQ ID NO: 50 - DNA, 324 bp, synthetic construct
  source 1..324: mol_type = other DNA
ctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccg

SEQ ID NO: 51 - DNA, 216 bp, synthetic construct
  source 1..216: mol_type = other DNA
tcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagat

SEQ ID NO: 52 - DNA, 887 bp, synthetic construct
  source 1..887: mol_type = other DNA
tctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcag

SEQ ID NO: 53 - DNA, 823 bp, synthetic construct
  source 1..823: mol_type = other DNA
actgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgt

SEQ ID NO: 54 - DNA, 12 bp, synthetic construct
  source 1..12: mol_type = other DNA
aggaagtctttt

SEQ ID NO: 55 - DNA, 2687 bp, synthetic construct
  source 1..2687: mol_type = other DNA
ccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttacaaaaaa

SEQ ID NO: 56 - DNA, 11058 bp, Homo sapiens
  source 1..11058: mol_type = unassigned DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagctgcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 57 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 58 - DNA, 11058 bp, Homo sapiens
  source 1..11058: mol_type = unassigned DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 59 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 60 - DNA, 8156 bp, synthetic construct
  source 1..8156: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacaaacgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgttaaccgtacgttttttttatcgctgccttgatatacactttccaccatgctgtggtgggaagaggtcgaagattgctacgaaagagaggacgtgcagaagaagacattcaccaagtgggtgaacgcccagttcagtaaatttggaaagcagcacatcgagaatctgttttcagacctgcaggatggccggagactgctggatctgctggaagggctgaccggacagaagctgcccaaagagaagggctctacacgcgtccatgcactgaacaatgtgaacaaagccctgcgagtcctgcagaacaataacgtggacctggtcaatatcgggagcactgacattgtggatggaaaccacaagctgaccctgggcctgatctggaatatcattctgcattggcaggtgaaaaatgtcatgaagaacatcatggccggcctgcagcagaccaacagcgagaaaattctgctgagctgggtgagacagtccacaaggaattacccccaagtgaatgtcatcaacttcaccacatcttggagtgacgggctggctctgaacgcactgattcacagccatagacctgacctgttcgattggaatagtgtggtctgccagcagtcagccacccagagactggagcacgcctttaacatcgctaggtatcagctgggcattgaaaagctgctggacccagaggacgtggatactacctaccccgacaagaaatccatcctgatgtatattacatctctgttccaggtcctgccacagcaggtgagcatcgaggccattcaggaagtggagatgctgcccaggccccctaaagtcaccaaggaggaacactttcagctgcaccatcagatgcattacagccagcagatcacagtgtccctggcacaggggtatgagcgaactagctccccaaaaccccggttcaagagctacgcctatactcaggccgcttacgtgacaacttccgatcccaccagaagccctttcccatcccagcacctggaagctcctgaggataagagctttggatctagtctgatggaatccgaggtgaacctggacaggtatcagactgccctggaggaagtcctgtcttggctgctgagtgcagaagacaccctgcaggcccagggcgaaatctccaacgatgtggaggtggtcaaggaccagtttcacacacatgaggggtacatgatggatctgactgcacaccagggccgcgtggggaatatcctgcagctgggctctaaactgattggaaccggcaagctgagtgaggacgaggaaacagaagtgcaggagcagatgaatctgctgaactctcgctgggaatgtctgcgagtggcctctatggagaagcagagtaatctgcaccgggtcctgatggatctgcagaatcagaaactgaaggagctgaacgactggctgaccaagacagaggaaagaaccaggaaaatggaggaagagcccctgggccctgacctggaagatctgaagcgacaggtccagcagcataaagtgctgcaggaggatctggaacaggagcaggtgcgggtcaacagcctgacacacatggtggtcgtggtcgatgaatcaagcggggaccatgccactgcagccctggaagagcagctgaaggtgctgggagacagatgggctaatatctgtcggtggaccgaggatagatgggtgctgctgcaggacattctgctgaaatggcagaggctgacagaagagcagtgcctgttctcagcctggctgagcgaaaaagaggatgctgtgaacaagatccacaccacagggtttaaggaccagaatgaaatgctgtcctctctgcagaaactggcagtgctgaaggccgatctggagaagaaaaagcagtctatgggaaaactgtacagtctgaagcaggacctgctgagcaccctgaaaaacaagagcgtgactcagaagaccgaagcttggctggataatttcgcaaggtgctgggacaacctggtgcagaaactggagaagtcaacagctcagatcagccaggctgcacctgggctgactaccattggagcaagcccaactcagaccgtgacactggtcactcagcccgtggtcacaaaagaaactgccatctccaagctggagatgcctagttcactgatgctggaactggagaggctgcaggaactgcaggaggcaaccgacgaactggatctgaagctgcgccaggccgaagtgatcaagggatcctggcagcccgtcggcgacctgctgattgattctctgcaggaccacctggagaaagtgaaggccctgagaggagaaatcgctcctctgaaggagaatgtgagccatgtcaacgatctggctaggcagctgacaactctgggcattcagctgtccccatataatctgtctactctggaggacctgaacacccggtggaaactgctgcaggtggccgtcgaagatcgcgtgcgacagctgcacgaggctcatagagacttcggcccagcatctcagcactttctgtcaacaagcgtgcaggggccctgggagagggctatcagtccaaataaggtgccctactatattaaccacgaaacacagaccacatgttgggatcatcctaaaatgactgagctgtaccagtcactggccgacctgaataacgtgaggtttagcgcttatcgcaccgcaatgaaactgaggcgcctgcagaaggccctgtgtctggatctgctgtccctgtctgccgcttgcgacgctctggatcagcacaatctgaagcagaacgatcagccaatggacatcctgcagatcattaactgcctgactaccatctacgaccgcctggaacaggagcataataacctggtgaatgtccccctgtgtgtggatatgtgcctgaattggctgctgaacgtctatgacaccggacgcacaggccggatcagagtgctgagtttcaagacaggcatcatttcactgtgtaaagcacacctggaggacaagtacagatacctgttcaagcaggtggcctccagcaccgggttttgcgatcagcgacggctgggactgctgctgcacgactccatccagattccccgccagctgggggaagtggcttccttcggcgggtctaacatcgagcctagtgtgcgatcatgtttccagtttgcaaataacaagccagaaattgaggcagccctgtttctggattggatgcggctggagcctcagagcatggtgtggctgccagtcctgcacagagtggctgcagccgaaaccgccaaacatcaggctaagtgcaacatctgtaaggagtgccctatcattggattcaggtaccgctccctgaaacatttcaactacgacatctgtcagagttgcttcttttcaggccgggtggctaaggggcacaagatgcattaccccatggtcgaatattgcactcctacaacttctggcgaggacgtgcgcgatttcgctaaggtcctgaaaaataagttccgaaccaaacggtactttgcaaagcatccacggatgggatacctgcccgtgcagacagtgctggagggggataatatggaaacagacacaatgtaagaagtcttttccacatggcagatgactcgagatcgatggcgcgccaccggtggctagtaataaaagatccttattttcattggatctgtgtgttggttttttgtgtgggtaccgtttgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 61 - protein, 1188 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAAPGLTTIGASPTQTVTLVTQPVVTKETAISKLEMPSSLMLELERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 62 - protein, 368 aa, synthetic construct
AHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 63 - DNA, 138 bp, synthetic construct
  source 1..138: mol_type = other DNA
atggccggcctgcagcagaccaacagcgagaaaattctgctgagctgggtgagacagtccacaaggaattacccccaagtgaatgtcatcaacttcaccacatcttggagtaatgggctgggactggacgcactgatt

SEQ ID NO: 64 - DNA, 138 bp, synthetic construct
  source 1..138: mol_type = other DNA
atggccggcctgcagcagaccaacagcgagaaaattctgctgagctgggtgagacagtccacaaggaattacccccaagtgaatgtcatcaacttcaccacatcttggagtgacgggacaggactgaacgcactgatt

SEQ ID NO: 65 - DNA, 138 bp, synthetic construct
  source 1..138: mol_type = other DNA
atggccggcctgcagcagaccaacagcgagaaaattctgctgagctgggtgagacagtccacaaggaattacccccaagtgaatgtcatcaacttcaccacatctctgagtgacgggacaggactgaacgcactgatt

SEQ ID NO: 66 - DNA, 8156 bp, synthetic construct; identical to SEQ ID NO: 60
  source 1..8156: mol_type = other DNA

SEQ ID NO: 67 - protein, 238 aa, synthetic construct
DSYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIE

SEQ ID NO: 68 - protein, 246 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIE

SEQ ID NO: 69 - protein, 20 aa, synthetic construct
FDWNSVVSQQSATQRLEHAF

SEQ ID NO: 70 - DNA, 60 bp, synthetic construct
  source 1..60: mol_type = other DNA
ttcgattggaatagtgtggtctgccagcagtcagccacccagagactggagcacgccttt

SEQ ID NO: 71 - DNA, 60 bp, synthetic construct
  source 1..60: mol_type = other DNA
ttcgattggaatagtgtggtctgccagaagtcagccaagtacagactggagcacgccttt

SEQ ID NO: 72 - DNA, 60 bp, synthetic construct
  source 1..60: mol_type = other DNA
ttcgattggaatagtgtggtccagcaggactcagccaagtacagactgcagcacgccttt

SEQ ID NO: 73 - DNA, 60 bp, synthetic construct
  source 1..60: mol_type = other DNA
ttcgattggaatagtgtggtcaagcaggactcagccaagtacagactgaagcacgccttt

SEQ ID NO: 74 - DNA, 60 bp, synthetic construct
  source 1..60: mol_type = other DNA
ttcgattgggacagtgtggtccagcagcagtcagccaagtacagactgcagcacgccttt

SEQ ID NO: 75 - DNA, 60 bp, synthetic construct
  source 1..60: mol_type = other DNA
ttcgattggaatagtgtggtcaaccagcagtcagcccagtacagactgaagcacgccttt

SEQ ID NO: 76 - DNA, 774 bp, synthetic construct
  source 1..774: mol_type = other DNA
acgcgtccatgcactgaacaatgtgaacaaagccctgcgagtcctgcagaacaataacgtggacctggtcaatatcgggagcactgacattgtggatggaaaccacaagctgaccctgggcctgatctggaatatcattctgcattggcaggtgaaaaatgtcatgaagaacatcatggccggcctgcagcagaccaacagcgagaaaattctgctgagctgggtgagacagtccacaaggaattacccccaagtgaatgtcatcaacttcaccacatcttggagtgacgggctggctctgaacgcactgattcacagccatagacctgacctgttcgattggaatagtgtggtctgccagcagtcagccacccagagactggagcacgcctttaacatcgctaggtatcagctgggcattgaaaagctgctggacccagaggacgtggatactacctaccccgacaagaaatccatcctgatgtatattacatctctgttccaggtcctgccacagcaggtgagcatcgaggccattcaggaagtggagatgctgcccaggccccctaaagtcaccaaggaggaacactttcagctgcaccatcagatgcattacagccagcagatcacagtgtccctggcacaggggtatgagcgaactagctccccaaaaccccggttcaagagctacgcctatactcaggccgcttacgtgacaacttccgatcccaccagaagccctttcccatcccagcacctggaagctcctgagga

(19) United States
(12) Patent Application Publication
(10) Pub. No.: US 2026/0226120 A1
(43) Pub. Date: Aug. 6, 2026

(54) ENGINEERED DYSTROPHIN PROTEINS WITH REDUCED IMMUNOGENICITY

(71) Applicant: UNIVERSITY OF WASHINGTON, Seattle, WA (US)

(72) Inventors: Jeffrey S. CHAMBERLAIN, Seattle, WA (US); Guy L. ODOM, Seattle, WA (US); Stephen D. HAUSCHKA, Seattle, WA (US); David BAKER, Seattle, WA (US); Indigo KING, Seattle, WA (US)

(73) Assignee: UNIVERSITY OF WASHINGTON, Seattle, WA (US)

(21) Appl. No.: 19/152,726

(22) PCT Filed: Feb. 15, 2024
(86) PCT No.: PCT/US2024/015971

§ 371 Date: Jul. 31, 2025

Related U.S. Application Data
(60) Provisional application No. 63/446,680, filed on Feb. 17, 2023.

(51) Int. Cl. C07K 14/47; A61K 38/00; A61P 21/00

(52) U.S. Cl. CPC C07K 14/4708; A61P 21/00; A61K 38/00

(57) ABSTRACT

The technology described herein relates to compositions and methods of treating a subject having muscular dystrophy.

### CROSS-REFERENCE TO RELATED APPLICATIONS

This application claims benefit under 35 U.S.C. § 119 (e) of U.S. Provisional Application No. 63/446,680 filed Feb. 17, 2023, the contents of which are incorporated herein by reference in their entirety.

### SEQUENCE LISTING

The instant application contains a Sequence Listing which has been submitted in XML format via Patent Center and is hereby incorporated by reference in its entirety. Said XML copy, created on Feb. 15, 2024, is named 034186-000100WOPT_SL.xml and is 129,900 bytes in size.

### TECHNICAL FIELD

The technology disclosed herein relates to therapeutic gene replacement.

### BACKGROUND

Duchenne muscular dystrophy (DMD) is a recessively-inherited muscle wasting disorder that affects approximately 1 in 3500 males. DMD patients carry a mutation in the dystrophin gene that causes aberrant expression or loss of expression of the dystrophin protein. DMD patients experience progressive wasting of skeletal muscles and cardiac dysfunction, which leads to loss of ambulation and premature death, primarily due to cardiac or respiratory failure.

Some patients with DMD have pre-existing anti-dystrophin T cells. Anti-dystrophin T cell immune responses have also been identified in the clinic following AAV-mediated gene therapy. Accordingly, there is an urgent need for compositions and methods for limiting or avoiding immune response to therapeutic dystrophin polypeptides in order to improve compositions and methods for treating DMD.

### SUMMARY

A problem in gene replacement approaches for muscular dystrophies is that the replacement dystrophin proteins tend to be recognized as non-self and provoke an immune response that limits therapeutic efficacy, duration or both. The technology described herein demonstrates that it is possible to significantly reduce immunogenicity of replacement dystrophin proteins using site-directed mutagenesis, while also maintaining efficient dystrophin activity.

In one aspect, described herein are recombinant human dystrophin polypeptides in which one or more T cell epitopes are modified to limit T cell recognition, while maintaining dystrophin function. In one embodiment of this or any other aspect, described herein is a recombinant human dystrophin polypeptide comprising one or more mutations in one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, wherein the recombinant polypeptide retains dystrophin activity.

In one embodiment of this or any other aspect described herein, the polypeptide is a full-length dystrophin polypeptide, a mini-dystrophin polypeptide, or a microdystrophin polypeptide.

In another embodiment of this or any other aspect described herein, the polypeptide comprises one or more mutations in Sequence Motif A that blunt T cell activity against a T cell epitope in Sequence Motif A.

In another embodiment of this or any other aspect described herein, the polypeptide comprises one or more mutations in Sequence Motif B that blunt T cell activity against a T cell epitope in Sequence Motif B.

In another embodiment of this or any other aspect described herein, the polypeptide comprises one or more mutations in Sequence Motif A and Sequence Motif B that blunt T cell activity against T cell epitopes in Sequence Motifs A and B.

In another embodiment of this or any other aspect described herein, the one or more mutations is/are substitution mutations.

In another embodiment of this or any other aspect described herein, the mutation in Sequence Motif A comprises amino acid changes at positions 167 and 168.

In another embodiment of this or any other aspect described herein, the mutation in Sequence Motif A consists of amino acid changes at positions 167 and 168.

In another embodiment of this or any other aspect described herein, the mutation at position 167 is L167T.

In another embodiment of this or any other aspect described herein, the mutation at position 168 is A168G.

In another embodiment of this or any other aspect described herein, the mutation at position 167 is L167T, and the mutation at position 168 is A168G.

In another embodiment of this or any other aspect described herein, the mutation in Sequence Motif A comprises amino acid changes at positions 163, 167 and 168.

In another embodiment of this or any other aspect described herein, the mutation in Sequence Motif A consists of amino acid changes at positions 163, 167 and 168.

In another embodiment of this or any other aspect described herein, the mutation at position 163 is W163L.

In another embodiment of this or any other aspect described herein, the mutation at position 167 is L167T.

In another embodiment of this or any other aspect described herein, the mutation at position 168 is A168G.

In another embodiment of this or any other aspect described herein, the mutation at position 163 is W163L, the mutation at position 167 is L167T, and the mutation at position 168 is A168G.

In another embodiment of this or any other aspect described herein, the mutation in Sequence Motif B comprises amino acid changes at positions 188, 190, 193, 194 and 197.

In another embodiment of this or any other aspect described herein, the polypeptide further comprises an amino acid change at position 184.

In another embodiment of this or any other aspect described herein, the mutation in Sequence Motif B consists of amino acid changes at positions 188, 190, 193, 194 and 197.

In another embodiment of this or any other aspect described herein, the mutation in Sequence Motif B consists of amino acid changes at positions 184, 188, 190, 193, 194 and 197.

In another embodiment of this or any other aspect described herein, the amino acid change at position 184 is N184D.

In another embodiment of this or any other aspect described herein, the amino acid change at position 188 is C188Q, C188K or C188N.

In another embodiment of this or any other aspect described herein, the amino acid change at position 190 is Q190D.

In another embodiment of this or any other aspect described herein, the amino acid change at position 193 is T193K.

In another embodiment of this or any other aspect described herein, the amino acid change at position 194 is Q194Y.

In another embodiment of this or any other aspect described herein, the amino acid change at position 197 is E197Q or E197K.

In another aspect, described herein is a recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9).

In another aspect, described herein is a recombinant human dystrophin polypeptide in which the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).

In another aspect, described herein is a recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9), and the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).

In one embodiment of this or any other aspect as described herein, the dystrophin polypeptide retains dystrophin activity.

In another embodiment of this or any other aspect described herein, the dystrophin polypeptide exhibits blunted T cell activity against the polypeptide relative to wild-type dystrophin.

In another aspect, described herein is a method of reducing T cell activity against a recombinant human dystrophin polypeptide, the method comprising mutating one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, and wherein the recombinant polypeptide retains dystrophin activity.

In one embodiment of this or any other aspect described herein, the recombinant human dystrophin polypeptide is a full-length dystrophin polypeptide, a mini-dystrophin polypeptide, or a microdystrophin polypeptide.

In another aspect, described herein is a nucleic acid encoding a human recombinant dystrophin polypeptide of any one of the described embodiments.

In another aspect, described herein is a vector comprising a nucleic acid encoding a human recombinant dystrophin polypeptide of any one of the embodiments described herein.

In one embodiment of this or any other aspect described herein, the vector is a plasmid or a viral vector.

In another embodiment of this or any other aspect described herein, the vector is a lentiviral vector, foamy viral vector, or an adeno-associated virus (AAV) vector.

In another aspect, described herein is a pharmaceutical formulation comprising a nucleic acid or a vector and a pharmaceutically acceptable carrier of any of the embodiments as described herein.

In another aspect, described herein is a cell comprising a recombinant human dystrophin polypeptide of any one of the embodiments, or a nucleic acid or vector encoding such polypeptide.

In another aspect, described herein is a method of increasing dystrophin activity in a cell, the method comprising introducing a nucleic acid or a vector of any one of the embodiments described herein to a cell.

In one embodiment of this or any other aspect described herein, the cell is a muscle cell.

In another aspect, described herein is a method of treating a muscular dystrophy in a subject in need thereof, the method comprising introducing a nucleic acid or a vector of any of the embodiments as described herein to a muscle cell in the subject.

In another embodiment of this or any other aspect described herein, the muscle cell is a skeletal muscle cell or a cardiac muscle cell.

In another embodiment of this or any other aspect described herein, the muscular dystrophy is myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, or an X-linked dilated cardiomyopathy.

In another aspect, described herein is a method of treating a muscular dystrophy in a subject in need thereof, the metho comprising administering a pharmaceutical formulation as described herein to the subject, whereby a nucleic acid encoding a recombinant human dystrophin polypeptide as described herein is introduced to a muscle cell of the subject.

In one embodiment of this or any other aspect described herein, the muscular dystrophy is myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, or an X-linked dilated cardiomyopathy.

### BRIEF DESCRIPTION OF THE DRAWINGS

FIG. 1A-1D shows a model of dystrophin indicating immunogenic epitopes of patient 5. FIG. 1A shows a computational generated illustration depicting exons 1-11 and its major functional domains. At left is the amino-terminal actin-binding domain (ABD) containing 3 actin-binding sites (ABS 1-3), formed within 2 adjacent calponin homology domains followed by hinge-1 which transitions into spectrin-like repeat 1 of the central rod domain. Within the ABD are the previously identified patient 5 dystrophin-specific epitopes found within exons 6-8 previously referred to as P17, P19 and P23. FIG. 1B depicts a schematic revealing a summarized view of protein domains, relative dystrophin-specific epitopes including amino acid positions, and characterized MHC haplotype for each epitope found to be immunoreactive within patient 5. FIG. 1C provides an overview of patient dosage information and relative immune response detected from the previous microdystrophin clinical trial (NCT00428935). FIG. 1D depicts domain structures of microdystrophins used in the current study (H3μDys) in addition to those being utilized in ongoing clinical trials of DMD delivered by rAAV.

FIGS. 2A-2K examines the redesign of the P17 epitope maintains structure and rescues pathophysiological indices in mdx^{4cv} mice. FIG. 2A shows a zoom view of human x-ray diffraction-based atomic data (1DXX), with a portion of the calponin-homology domain displayed with redesign structural predictions and modified amino acids shown as designs (Des-1,-2,-3). The redesigned P17 epitope is predicted not to bind to HLA-DQA10505. FIG. 2B depicts embedded table showing redesign(s) of the native core epitope sequence with predicted IC50 (concentration for 50% MHC binding) and ROSETTA energy unit (REU) predictions for WSDGLALNA (native HLA-DQ7 epitope, SEQ ID NO: 3), WSNGLGLDA (Design-1, SEQ ID NO: 4), WSDGTGLNA (Design-2, SEQ ID NO: 5), and LSDGTGLNA (Design-3, SEQ ID NO: 6). FIG. 2C shows a western blot analysis of clonally derived mdx myotube cultures demonstrate robust expression at 4-days post-differentiation for Des-2 and Des-3 while Des-1 yields an ⅔ reduction relative to native μDys. FIG. 2D examines immunostaining of gastrocnemius muscle for μDys and adjacent H&E staining revealing central nucleation and inflammatory infiltrate in untreated and treated mice. Determination of physiological performance from gastrocnemius muscle 8 months post-treatment with specific force generation (FIG. 2E), maximum tetanic force (FIG. 2F), resistance to contraction induced injury (FIG. 2G), and recovery force 1 minute following injury assay (FIG. 2H). Whole diaphragm contractile function was assessed noninvasively by high resolution sonography (FIG. 2I), while at end point specific force was determined using in vitro diaphragm strip contractile assays (FIG. 2J). FIG. 2K shows western blot results of gastrocnemius tissue lysates indicating detection of μDys protein with GAPDH as the loading control.

FIGS. 3A-3E shows in vitro and in vivo assessment of rAAV6 vectors encoding P19 redesigns improve hindlimb function in mdx^{4cv} mice. FIG. 3A shows native Dys and corresponding P19 epitope redesign mutations are displayed. FIG. 3B depicts a display of relative mutation positions for the P19 redesigns tested: RPDLFDWNSVVSQQSATQRLEHAFNIAR (SEQ ID NO: 10), RPDLFDWNSVVSQKSAKYRLEHAFNIAR (SEQ ID NO: 11), RPDLFDWNSVVQQDSAKYRLQHAFNIAR (SEQ ID NO: 12), RPDLFDWNSVVKQDSAKYRLKHAFNIAR (SEQ ID NO: 13), RPDLFDWDSVVQQQSAKYRLQHAFNIAR (SEQ ID NO: 14), RPDLFDWNSVVNQQSAQYRLKHAFNIAR (SEQ ID NO: 15). FIG. 3C shows a western blot assay of P19 redesigns following transduction with rAAV-CK8e-Dys mutants (m1-m5). Displayed below, mdx myotubes transduced with rAAV6-CK8e-mCherry control vector as imaged just prior to harvest (4-day). FIG. 3D displays hindlimb torque assays reveal distinct differences in the restoration of force production. FIG. 3E examines representative immunofluorescent imaging of gastrocnemius cryosections labeled with dystrophin, B2 laminin antibodies, followed by DAPI to label nuclei.

FIGS. 4A-4B examines the deimmunization of P17 and P19 epitopes demonstrated by interferon-gamma enzyme-linked immunosorbent spot (ELISpot) assays. FIG. 4A shows native peptide P17 (amino acids 161 through 180, exon 6) in parallel to incubation (1 μg/ml/2 hrs) with redesign variants (Des-1-3) were used to evaluate epitope presentation, T cell activation and resultant detected production of inf-γ in the form of counted spots. As autologous APC's, a transformed B lymphoblastoid cell line (BLCL) was used. Each of the P17 redesigns found to be deimmunized or a lack of inf-γ production from patient 5 T cells. Background levels (dashed line) varied based on the nature of the response established from the patients' T cells. A positive response being defined as 3-fold above background (solid line). FIG. 4B depicts in a similar fashion, inf-γ ELISpot was performed using native peptide P19 (amino acids 181-200, exon 7) in parallel with 5 (P19) redesign candidates (M1-M5). Here, 4 of 5 redesign candidates were effectively deimmunized or resulted in inf-γ spot counts below background.

FIGS. 5A-5E shows combined dual epitope (P17/P19) redesigns improves cardio-respiratory performance in mdx^{4cv} mice. FIG. 5A shows following systemic administration of vectors, global diaphragm function was assessed by high resolution sonography at 2, 4,8 months post-treatment. FIG. 5B depicts cryosections processed for immunofluorescent imaging demonstrating the localization of dystrophin, laminin-2, and nuclei within cross sections of diaphragm and heart (size bar=20 μm). FIG. 5C-5D displays cardiac MRI determination of ejection fraction (FIG. 5C) and stroke volume (FIG. 5D) in 4 cohorts of aged mice following treatment at 2-weeks of age: C57BL/6, untreated mdx^{4cv} mice and mdx^{4cv} mice treated dual epitope redesign (D2M1 or D2M2) vectors. FIG. 5E examines extracellular volume (ECV), linearly related with fibrosis volume in myocardium, estimated by Tl mapping with Gd-DTPA injections.

FIG. 6 is a schematic showing exemplary microdystrophins used in the clinic.

FIG. 7 is a series of blots and images showing testing of Hinge3 μDys P17 redesigns.

FIG. 8 is a series of blots and images showing testing of Hinge3 μDys P19 redesigns.

FIG. 9 is a series of graphs showing testing of the Hinge3 μDys P19 redesign #1; the redesigns retained functionality in assessments of specific force and force injury.

FIG. 10 is a series of images showing testing of the combination of P17 #2 and P19 #1, with 9 amino acids between the P17 and P19 epitopes.

FIG. 11 is a series of graphs showing testing of the combination of P17 #2 and P19 #1, which retained functionality in assessments of specific force and force injury.

FIG. 12 is a series of graphs showing testing of P17/P19 combinations; the P17 #2/P19 #1 combination retained functionality in cardiac assessments.

FIG. 13 is a bar graph showing testing of Hinge3 μDys P17 redesigns for immune cell responses.

FIG. 14 is a bar graph showing testing of Hinge3 μDys P19 redesigns for immune cell responses.

### DETAILED DESCRIPTION

The technology described herein relates to compositions and methods for muscular dystrophy gene replacement therapy, and include administering a modified dystrophin (whether full length, mini- or micro-dystrophin) gene and a delivery vehicle to a subject. The coding sequence for dystrophin polypeptides are modified to reduce immune recognition of the therapeutic polypeptides while maintaining dystrophin function. The following provides considerations for one of ordinary skill in the art to practice the technology disclosed herein.

### Dystrophin

Dystrophin is a 427 kDa cytoskeletal protein and is a member of the spectrin/a-actinin superfamily (See e.g., Blake et al., Brain Pathology, 6:37 (1996); Winder, J. Muscle Res. Cell. Motil., 18:617 (1997); and Tinsley et al., PNAS, 91:8307 (1994)). The N-terminus of dystrophin binds to actin, having a higher affinity for non-muscle actin than for sarcomeric actin. Dystrophin is involved in the submembranous network of non-muscle actin underlying the plasma membrane. Dystrophin is associated with an oligomeric, membrane spanning complex of proteins and glycoproteins, the dystrophin-associated protein complex (DGC). The C-terminus of dystrophin binds to the cytoplasmic tail of B-dystroglycan, and in concert with actin, anchors dystrophin to the sarcolemma. Also bound to the C-terminus of dystrophin are the cytoplasmic members of the DGC. Dystrophin thereby provides a link between the actin-based cytoskeleton of the muscle fiber and the extracellular matrix. It is this link that is disrupted in muscular dystrophy.

The central rod domain of dystrophin is composed of a series of 24 weakly repeating units of approximately 110 amino acids, similar to those found in spectrin (i.e., spectrin-like repeats). This domain constitutes the majority of dystrophin and gives dystrophin a flexible rod-like structure. The rod-domain is interrupted by four hinge regions that are rich in proline. It is contemplated that the rod-domain provides a structural link between members of the DGC.

TABLE 1
Exemplary Approximate Domains of Full-length dystrophin - nucleic acid (see
e.g., NCBI Accession No. XM_006724470.4)

| Exemplary | | | |
|---|---|---|---|
| Approximate | | SEQ ID | |
| Nucleotides | Domain | NO: | Exemplary Sequence |
| 1-208 | 5′ | SEQ ID | atcagttact gtgttgactc actcagtgtt gggatcactc actttccccc |
| | untranslated | NO: | tacaggactcagatctggga ggcaattacc ttcggagaaa |
| | region | 21 | aacgaatagg aaaaactgaa gtgttactttttttaaagct |
| | | | gctgaagttt gttggtttct cattgttttt aagcctactg |
| | | | gagcaataaagtttgaagaa cttttaccag gtttttttt |
| 209-211 | Start codon | | — |
| | (e.g., ATC, | | |
| | ATG) | | |
| 209-964 | N terminus | SEQ ID | gctgcctt gatatacact tttcaaaatgctttcgtggg aagaagtaga |
| | (e.g., ABD) | NO: | ggactgttat gaaagagaag atgttcaaaa |
| | | 22 | gaaaacattcacaaaatggg taaatgcaca attttctaag |
| | | | tttgggaagc agcatattga gaacctcttcagtgacctac |
| | | | aggatgggag gcgcctccta gacctcctcg aaggcctgac |
| | | | agggcaaaaactgccaaaag aaaaaggatc cacaagagtt |
| | | | catgccctga acaatgtcaa caaggcactgcgggttttgc |
| | | | agaacaataa tgttgattta gtgaatattg gaagtactga |
| | | | catcgtagatggaaatcata aactgactct tggtttgatt |
| | | | tggaatataa tcctccactg gcaggtcaaaaatgtaatga |
| | | | aaaatatcat ggctggattg caacaaacca acagtgaaaa |
| | | | gattctcctgagctgggtcc gacaatcaac tcgtaattat |
| | | | ccacaggtta atgtaatcaa cttcaccaccagctggtctg |
| | | | atggcctggc tttgaatgct ctcatccata gtcataggcc |
| | | | agacctatttgactggaata gtgtggtttg ccagcagtca |
| | | | gccacacaac gactggaaca tgcattcaacatcgccagat |
| | | | atcaattagg catagagaaa ctactcgatc ctgaagatgt |
| | | | tgataccacctatccagata agaagtccat cttaatgtac |
| | | | atcacatcac tcttccaagt tttgcctcaacaag |
| 965-1219 | Hinge 1 | SEQ ID | tgagca ttgaagccat ccaggaagtg gaaatgttgc |
| | | NO: | caaggccacc taaagtgactaaagaagaac attttcagtt |
| | | 23 | acatcatcaa atgcactatt ctcaacagat |
| | | | cacggtcagtctagcacagg gatatgagag aacttcttcc |
| | | | cctaagcctc gattcaagag ctatgcctacacacaggctg |
| | | | cttatgtcac cacctctgac cctacacgga gcccatttcc |
| | | | ttcacagcatttggaagctc ctgaagaca |
| 1220-1546 | Spectrin-like | SEQ ID | a gtcatttggc agttcattga tggagagtga |
| | repeat No. 1 | NO: | agtaaacctggaccgttatc aaacagcttt agaagaagta |
| | | 24 | ttatcgtggc ttctttctgc tgaggacacattgcaagcac |
| | | | aaggagagat ttctaatgat gtggaagtgg tgaaagacca |
| | | | gtttcatactcatgaggggt acatgatgga tttgacagcc |
| | | | catcagggcc gggttggtaa tattctacaattgggaagta |
| | | | agctgattgg aacaggaaaa ttatcagaag atgaagaaac |
| | | | tgaagtacaagagcagatga atctcctaaa ttcaagatgg |
| | | | gaatgcctca gggtag |
| 1547-1879 | Spectrin-like | SEQ ID | ctag catggaaaaacaaagcaatt tacatagagt tttaatggat |
| | repeat No. 2 | NO: | ctccagaatc agaaactgaa agagttgaat gactggctaa |
| | | 25 | caaaaacaga agaaagaaca aggaaaatgg aggaagagcc |
| | | | tcttggacctgatcttgaag acctaaaacg ccaagtacaa |
| | | | caacataagg tgcttcaaga agatctagaacaagaacaag |
| | | | tcagggtcaa ttctctcact cacatcgtcg tggtagttga |
| | | | tgaatctagtggagatcacg caactgctgc tttggaagaa |
| | | | caacttaagg tattgggaga tcgatgggcaaacatctgta |
| | | | gatggacag |
| 1880-2212 | Spectrin-like | SEQ ID | a agaccgctgg gttcttttac aagacatcct |
| | repeat No. 3 | NO: 26 | tctcaaatggcaacgtctta ctgaagaaca gtgccttttt |
| | | | agtgcatggc tttcagaaaa agaagatgcagtgaacaaga |
| | | | ttcacacaac tggctttaaa gatcaaaatg aaatgttatc |
| | | | aagtcttcaaaaactggccg ttttaaaagc ggatctagaa |
| | | | aagaaaaagc aatccatggg caaactgtattcactcaaac |
| | | | aagatcttct ttcaacactg aagaataagt cagtgaccca |
| | | | gaagacggaagcatggctgg ataactttgc ccggtgttgg |
| | | | gataatttag tccaaaaact tg |
| 2213-2359 | Hinge 2 | SEQ ID | aaaagagtacagcacaga tttcacaggc tgtcaccacc |
| | | NO: 27 | actcagccat cactaacaca gacaactgtaatggaaacag |
| | | | taactacggt gaccacaagg gaacagatcc tggtaaagca |
| | | | tgctcaagaggaacttccac caccacctc |
| 2360-2692 | Spectrin-like | SEQ ID | c ccaaaagaag aggcagatta ctgtggattc |
| | repeat No. 4 | NO: 28 | tgaaattaggaaaaggttgg atgttgatat aactgaactt |
| | | | cacagctgga tactcgctc agaagctgtgttgcagagtc |
| | | | ctgaatttgc aatctttcgg aaggaaggca acttctcaga |
| | | | cttaaaagaaaaagtcaatg ccatagagcg agaaaaagct |
| | | | gagaagttca gaaaactgca agatgccagcagatcagctc |
| | | | aggccctggt ggaacagatg gtgaatgagg gtgttaatgc |
| | | | agatagcatcaaacaagcct cagaacaact gaacagccgg |
| | | | tggatcgaat tctgccagtt gc |
| 2693-3019 | Spectrin-like | SEQ ID | taagtgagagacttaact ggctggagta tcagaacaac |
| | repeat No. 5 | NO: 29 | atcatcgctt tctataatca gctacaacaattggagcaga |
| | | | tgacaactac tgctgaaaac tcgttgaaaa tccaacccac |
| | | | caccccatcagagccaacag caattaaaag tcagttaaaa |
| | | | atttgtaagg atgaagtcaa ccggctatcagatcttcaac |
| | | | ctcaaattga acgattaaaa attcaaagca tagccctgaa |
| | | | agagaaaggacaaggaccca tgttcctgga tgcagacttt |
| | | | gtggccttta caaatcattt taagcaagtcttttctgatg tgcaggcca |
| 3020-3346 | Spectrin-like | SEQ ID | g agagaaagag ctacagacaa tttttgacac |
| | repeat No. 6 | NO: 30 | tttgccaccaatggctatc aggagaccat gagtgccatc |
| | | | aggacatggg tccagcagtc agaaaccaaactctccatac |
| | | | ctcaacttag tgtcaccgac tatgaaatca tggagcagag |
| | | | actcggggaattgcaggctt tacaaagttc tctgcaagag |
| | | | caacaaagtg gcctatacta tctcagcaccactgtgaaag |
| | | | agatgtcgaa gaaagcgccc tctgaaatta gccggaaata |
| | | | tcaatcagaatttgaagaaa ttgagggacg ctggaagaag |
| | | | ctctcctccc agctgg |
| 3347-3673 | Spectrin-like | SEQ ID | ttga gcattgtcaaaagctagagg agcaaatgaa taaactccga |
| | repeat No. 7 | NO: 31 | aaaattcaga atcacataca aaccctgaagaaatggatgg |
| | | | ctgaagttga tgtttttctg aaggaggaat ggcctgccct |
| | | | tggggattcagaaattctaa aaaagcagct gaaacagtgc |
| | | | agacttttag tcagtgatat tcagacaattcagcccagtc |
| | | | taaacagtgt caatgaaggt gggcagaaga taaagaatga |
| | | | agcagagccagagtttgctt cgagacttga gacagaactc |
| | | | aaagaactta acactcagtg ggatcacatgtgccaacagg tct |
| 3674-4000 | Spectrin-like | SEQ ID | atgccag aaaggaggcc ttgaagggag gtttggagaa |
| | repeat No. 8 | NO: 32 | aactgtaagcctccagaaag atctatcaga gatgcacgaa |
| | | | tggatgacac aagctgaaga agagtatcttgagagagatt |
| | | | ttgaatataa aactccagat gaattacaga aagcagttga |
| | | | agagatgaagagagctaaag aagaggccca acaaaaagaa |
| | | | gcgaaagtga aactccttac tgagtctgtaaatagtgtca |
| | | | tagctcaagc tccacctgta gcacaagagg ccttaaaaaa |
| | | | ggaacttgaaactctaacca ccaactacca gtggctctgc |
| | | | actaggctga |
| 4001-4312 | Spectrin-like | SEQ ID | atgggaaatg caagactttggaagaagttt gggcatgttg |
| | repeat No. 9 | NO: 33 | gcatgagtta ttgtcatact tggagaaagc |
| | | | aaacaagtggctaaatgaag tagaatttaa acttaaaacc |
| | | | actgaaaaca ttcctggcgg agctgaggaaatctctgagg |
| | | | tgctagattc acttgaaaat ttgatgcgac attcagagga |
| | | | taacccaaatcagattcgca tattggcaca gaccctaaca |
| | | | gatggcggag tcatggatga gctaatcaatgaggaacttg |
| | | | agacatttaa ttctcgttgg agggaactac atgaagaggc tg |
| 4313-4588 | Spectrin-like | SEQ ID | taaggaggcaaaagttgc ttgaacagag catccagtct |
| | repeat No. | NO: 34 | gcccaggaga ctgaaaaatc cttacacttaatccaggagt |
| | 10 | | ccctcacatt cattgacaag cagttggcag cttatattgc |
| | | | agacaaggtggacgcagctc aaatgcctca ggaagcccag |
| | | | aaaatccaat ctgatttgac aagtcatgagatcagtttag |
| | | | aagaaatgaa gaaacataat caggggaagg aggctgccca |
| | | | aagagtcctgtctcagattc atgttgcaca gaaaaaat |
| 4589-4915 | Spectrin-like | SEQ ID | ta caagatgtct ccatgaagtt tcgattattccagaaaccag |
| | repeat No. | NO: 35 | ccaattttga gcagcgtcta caagaaagta agatgatttt |
| | 11 | | agatgaagtgaagatgcact tgcctgcatt ggaaacaaag |
| | | | agtgtggaac aggaagtagt acagtcacagctaaatcatt |
| | | | gtgtgaactt gtataaaagt ctgagtgaag tgaagtctga |
| | | | agtggaaatggtgataaaga ctggacgtca gattgtacag |
| | | | aaaaagcaga cggaaaatcc caaagaacttgatgaaagag |
| | | | taacagcttt gaaattgcat tataatgagc tgggagcaaa ggtaa |
| 4916-5239 | Spectrin-like | SEQ ID | cagaaagaaagcaac agttggagaa atgcttgaaa ttgtcccgta |
| | repeat No. | NO: 36 | agatgcgaaa ggaaatgaatgtcttgacag aatggctggc |
| | 12 | | agctacagat atggaattga caaagagatc |
| | | | agcagttgaaggaatgccta gtaatttgga ttctgaagtt |
| | | | gcctggggaa aggctactca aaaagagattgagaaacaga |
| | | | aggtgcacct gaagagtatc acagaggtag gagaggcctt |
| | | | gaaaacagttttgggcaaga aggagacctt ggtggaagat |
| | | | aaactcagtc ttctgaatag taactggatagctgtcacct |
| | | | cccgagcag |
| 5340-5551 | Spectrin-like | SEQ ID | a agagtggtta aatcttttgt tggaatacca |
| | repeat No. | NO: 37 | gaaacacatggaaacttttg accagaatgt ggaccacatc |
| | 13 | | acaaagtgga tcattcaggc tgacacacttttggatgaat |
| | | | cagagaaaaa gaaaccccag caaaaagaag acgtgcttaa |
| | | | gcgtttaaaggcagaactga atgacatacg cccaaaggtg |
| | | | gactctacac gtgaccaagc a |
| 5552-5833 | Spectrin-like | SEQ ID | gcaaacttgatggcaaacc gcggtgacca ctgcaggaaa |
| | repeat No. | NO: 38 | ttagtagagc cccaaatctc agagctcaaccatcgatttg |
| | 14 | | cagccatttc acacagaatt aagactggaa aggcctccat |
| | | | tcctttgaaggaattggagc agtttaactc agatatacaa |
| | | | aaattgcttg aaccactgga ggctgaaattcagcaggggg |
| | | | tgaatctgaa agaggaagac ttcaataaag atatgaatga |
| | | | agacaatgagggtactgtaa aagaattgtt gcaaagagga |
| | | | gacaacttac aacaaagaat cacagatgagagaaagcgag |
| | | | aggaaataaa gataaaacag cagctgttac agacaaaaca |
| | | | taatgctctcaaggatttga ggt |
| 5834-6127 | Spectrin-like | SEQ ID | ctcaaag aagaaaaaag gctctagaaa tttctcatca |
| | repeat No. | NO: 39 | gtggtatcagtacaagaggc aggctgatga tctcctgaaa |
| | 15 | | tgcttggatg acattgaaaa aaaattagccagcctacctg |
| | | | agcccagaga tgaaaggaaa ataaaggaaa ttgatcggga |
| | | | attgcagaagaagaaagagg agctgaatgc agtgcgtagg |
| | | | caagctgagg gcttgtctga ggatggggccgcaatggcag |
| | | | tggagccaac tcagatccag ctcagcaagc gctggggga |
| | | | aattgagagcaaatttg |
| 6128-6187 | 20 amino | SEQ ID | ctcagtttcgaag actcaacttt gcacaaattc acactgtccg |
| | acid insert | NO: 40 | tgaagaaacgatgatgg |
| | (not hinge) | | |
| 6188-6514 | Spectrin-like | SEQ ID | tga tgactgaaga catgcctttg gaaatttctt atgtgccttc |
| | repeat No. | NO: 41 | tacttatttgactgaaatca ctcatgtctc acaagcccta |
| | 16 | | ttagaagtgg aacaacttct caatgctcctgacctctgtg |
| | | | ctaaggactt tgaagatctc tttaagcaag aggagtctct |
| | | | gaagaatataaaagatagtc tacaacaaag ctcaggtcgg |
| | | | attgacatta ttcatagcaa gaagacagcagcattgcaaa |
| | | | gtgcaacgcc tgtggaaagg gtgaagctac aggaagctct |
| | | | ctcccagcttgatttccaat gggaaaaagt taacaaaatg taca |
| 6515-6835 | Spectrin-like | SEQ ID | aggacc gacaagggcg atttgacagatctgttgaga |
| | repeat No. | NO: 42 | aatggcggcg ttttcattat gatataaaga tatttaatca |
| | 17 | | gtggctaacagaagctgaac agtttctcag aaagacacaa |
| | | | attcctgaga attgggaaca tgctaaatacaaatggtatc |
| | | | ttaaggaact ccaggatggc attgggcagc ggcaaactgt |
| | | | tgtcagaacattgaatgcaa ctggggaaga aataattcag |
| | | | caatcctcaa aaacagatgc cagtattctacaggaaaaat |
| | | | tgggaagcct gaatctgcgg tggcaggagg tctgcaaaca gctgt |
| 6836-7186 | Spectrin-like | SEQ ID | cagacagaaaaaaga ggctagaaga acaaaagaat |
| | repeat No. | NO: 43 | atcttgtcag aatttcaaag agatttaaatgaatttgttt tatggttgga |
| | 18 | | ggaagcagat aacattgcta gtatcccact |
| | | | tgaacctggaaaagagcagc aactaaaaga aaagcttgag |
| | | | caagtcaagt tactggtgga agagttgcccctgcgccagg |
| | | | gaattctcaa acaattaaat gaaactggag gacccgtgct |
| | | | tgtaagtgctcccataagcc cagaagagca agataaactt |
| | | | gaaaataagc tcaagcagac aaatctccagtggataaagg |
| | | | tttccagagc tttacctgag aaacaaggag aaattg |
| 7187-7489 | Spectrin-like | SEQ ID | aagc tcaaataaaagaccttgggc agcttgaaaa aaagcttgaa |
| | repeat No. | NO: 44 | gaccttgaag agcagttaaa tcatctgctgctgtggttat ctcctattag |
| | 19 | | gaatcagttg gaaatttata accaaccaaa |
| | | | ccaagaaggaccatttgacg ttaaggaaac tgaaatagca |
| | | | gttcaagcta aacaaccgga tgtggaagagattttgtcta |
| | | | aagggcagca tttgtacaag gaaaaaccag ccactcagcc |
| | | | agtgaagaggaagttagaag atctgagctc tgagtggaag |
| | | | gcggtaaacc gtttacttc |
| 7490-7612 | Hinge 3 | SEQ ID | a agagctgagggcaaagcagc ctgacctagc tcctggactg |
| | | NO: 45 | accactattg gagcctctcc tactcagactgttactctgg |
| | | | tgacacaacc tgtggttact aaggaaactg ccatctccaa ac |
| 7613-7942 | Spectrin-like | SEQ ID | tagaaatgccatcttcct tgatgttgga ggtacctgct ctggcagatt |
| | repeat No. | | tcaaccgggc ttggacagaacttaccgact ggctttctct |
| | | | gcttgatcaa gttataaaat cacagagggt |
| | | | gatggtgggtgaccttgagg atatcaacga gatgatcatc |
| | | | aagcagaagg caacaatgca ggatttggaa |
| | 20 | NO: 46 | cagaggcgtc cccagttgga agaactcatt accgctgccc |
| | | | aaaatttgaa aaacaagaccagcaatcaag aggctagaac |
| | | | aatcattacg gatcgaattg aaagaattca |
| | | | gaatcagtgggatgaagtac aagaacacct tc |
| 7943-8269 | Spectrin-like | SEQ ID | agaaccgg aggcaacagt tgaatgaaat |
| | repeat No. | NO: 47 | gttaaaggattcaacacaat ggctggaagc taaggaagaa |
| | 21 | | gctgagcagg tcttaggaca ggccagagccaagcttgagt |
| | | | catggaagga gggtccctat acagtagatg caatccaaaa |
| | | | gaaaatcacagaaaccaagc agttggccaa agacctccgc |
| | | | cagtggcaga caaatgtaga tgtggcaaatgacttggccc |
| | | | tgaaacttct ccgggattat tctgcagatg ataccagaaa |
| | | | agtccacatgataacagaga atatcaatgc ctcttggaga |
| | | | agcattcata aaagggtga |
| 8270-8617 | Spectrin-like | SEQ ID | g tgagcgagaggctgctttgg aagaaactca tagattactg |
| | repeat No. | NO: 48 | caacagttcc ccctggacct ggaaaagtttcttgcctggc |
| | 22 | | ttacagaagc tgaaacaact gccaatgtcc tacaggatgc |
| | | | tacccgtaaggaaaggctcc tagaagactc caagggagta |
| | | | aaagagctga tgaaacaatg gcaagacctccaaggtgaaa |
| | | | ttgaagctca cacagatgtt tatcacaacc tggatgaaaa |
| | | | cagccaaaaaatcctgagat ccctggaagg ttccgatgat |
| | | | gcagtcctgt tacaaagacg tttggataacatgaacttca |
| | | | agtggagtga acttcggaaa aagtctc |
| 8618-9004 | Spectrin-like | SEQ ID | tca acattaggtc ccatttggaagccagttctg accagtggaa |
| | repeat No. | NO: 49 | gcgtctgcac ctttctctgc aggaacttct |
| | 23 | | ggtgtggctacagctgaaag atgatgaatt aagccggcag |
| | | | gcacctattg gaggcgactt tccagcagttcagaagcaga |
| | | | acgatgtaca tagggccttc aagagggaat tgaaaactaa |
| | | | agaacctgtaatcatgagta ctcttgagac tgtacgaata |
| | | | tttctgacag agcagccttt ggaaggactagagaaactct |
| | | | accaggagcc cagagagctg cctcctgagg agagagccca |
| | | | gaatgtcactcggcttctac gaaagcaggc tgaggaggtc |
| | | | aatactgagt gggaaaaatt gaacctgcactccg |
| 9005-9328 | Spectrin-like | SEQ ID | ctgact ggcagagaaa aatagatgag acccttgaaa |
| | repeat No. | NO: 50 | gactccggga acttcaagaggccacggatg agctggacct |
| | 24 | | caagctgcgc caagctgagg tgatcaaggg |
| | | | atcctggcagcccgtgggg atctcctcat tgactctctc |
| | | | caagatcacc tcgagaaagt caaggcacttcgaggagaaa |
| | | | ttgcgcctct gaaagagaac gtgagccacg tcaatgacct |
| | | | tgctcgccagcttaccactt tgggcattca gctctcaccg |
| | | | tataacctca gcactctgga agacctgaacaccagatgga |
| | | | agcttctgca ggtggccg |
| 9329-9544 | Hinge 4 | SEQ ID | tc gaggaccgag tcaggcagct gcatgaagcccacagggact |
| | | NO: 51 | ttggtccagc atctcagcac tttctttcca cgtctgtcca |
| | | | gggtccctgggagagagcca tctcgccaaa caaagtgccc |
| | | | tactatatca accacgagac tcaaacaacttgctgggacc |
| | | | atcccaaaat gacagagctc taccagtctt tagctgacct |
| | | | gaataatgtcagat |
| 9545-10431 | Start of C | SEQ ID | tctcag cttataggac tgccatgaaa ctccgaagac tgcagaaggc |
| | terminus | NO: 52 | cctttgcttggatctcttga gcctgtcagc tgcatgtgat gccttggacc |
| | | | agcacaacct caagcaaaatgaccagccca tggatatcct |
| | | | gcagattatt aattgtttga ccactattta |
| | | | tgaccgcctggagcaagagc acaacaattt ggtcaacgtc |
| | | | cctctctgcg tggatatgtg tctgaactggctgctgaatg tttatgatac |
| | | | gggacgaaca gggaggatcc gtgtcctgtc |
| | | | ttttaaaactggcatcattt ccctgtgtaa agcacatttg |
| | | | gaagacaagt acagatacct tttcaagcaagtggcaagtt |
| | | | caacaggatt ttgtgaccag cgcaggctgg gcctccttct |
| | | | gcatgattctatccaaattc caagacagtt gggtgaagtt |
| | | | gcatcctttg ggggcagtaa cattgagccaagtgtccgga |
| | | | gctgcttcca atttgctaat aataagccag agatcgaagc |
| | | | ggccctcttcctagactgga tgagactgga accccagtcc |
| | | | atggtgtggc tgcccgtcct gcacagagtggctgctgcag |
| | | | aaactgccaa gcatcaggcc aaatgtaaca tctgcaaaga |
| | | | gtgtccaatcattggattca ggtacaggag tctaaagcac |
| | | | tttaattatg acatctgcca aagctgctttttttctggtc gagttgcaaa |
| | | | aggccataaa atgcactatc ccatggtgga |
| | | | atattgcactccgactacat caggagaaga tgttcgagac |
| | | | tttgccaagg tactaaaaaa caaatttcgaaccaaaaggt |
| | | | attttgcgaa gcatccccga atgggctacc tgccagtgca g |
| 10432-11254 | Alternatively | SEQ ID | actgtcttagagggggaca acatggaaac gcctgcctg |
| | spliced | NO: 53 | tcccctcagc tttcacacga tgatactcattcacgcattg |
| | exons 71-78 | | aacattatgc tagcaggcta gcagaaatgg aaaacagcaa |
| | | | tggatcttatctaaatgata gcatctctcc taatgagagc |
| | | | atagatgatg aacatttgtt aatccagcattactgccaaa |
| | | | gtttgaacca ggactccccc ctgagccagc ctcgtagtcc |
| | | | tgcccagatcttgatttcct tagagagtga ggaaagaggg |
| | | | gagctagaga gaatcctagc agatcttgaggaagaaaaca |
| | | | ggaatctgca agcagaatat gaccgtctaa agcagcagca |
| | | | cgaacataaaggcctgtccc cactgccgtc ccctcctgaa |
| | | | atgatgccca cctctcccca gagtccccgggatgctgagc |
| | | | tcattgctga ggccaagcta ctgcgtcaac acaaaggccg |
| | | | cctggaagccaggatgcaaa tcctggaaga ccacaataaa |
| | | | cagctggagt cacagttaca caggctaaggcagctgctgg |
| | | | agcaacccca ggcagaggcc aaagtgaatg gcacaacggt |
| | | | gtcctctccttctacctctc tacagaggtc cgacagcagt |
| | | | cagcctatgc tgctccgagt ggttggcagtcaaacttcgg |
| | | | actccatggg tgaggaagat cttctcagtc ctccccagga |
| | | | cacaagcacagggttagagg aggtgatgga gcaactcaac |
| | | | aactccttcc ctagttcaag aggaagaaatacccctggaa |
| | | | agccaatgag agaggacaca atgt |
| 11255-11266 | End of | SEQ ID | aggaag tctttt |
| | Coding | NO: 54 | |
| | Region | | |
| 11267-13957 | 3 | SEQ ID | ccac atggcagatgatttgggcag agcgatggag tccttagtat |
| | untranslated | NO: 55 | cagtcatgac agatgaagaa ggagcagaataaatgtttta |
| | region | | caactcctga ttcccgcatg gtttttataa tattcataca |
| | | | acaaagaggattagacagta agagtttaca agaaataaat |
| | | | ctatattttt gtgaagggta gtggtattatactgtagatt tcagtagttt |
| | | | ctaagtctgt tattgttttg ttaacaatgg caggttttacacgtctatgc |
| | | | aattgtacaa aaaagttata agaaaactac atgtaaaatc |
| | | | ttgatagctaaataacttgc catttcttta tatggaacgc attttgggtt |
| | | | gtttaaaaat ttataacagttataaagaaa gattgtaaac |
| | | | taaagtgtgc tttataaaaa aaagttgttt |
| | | | ataaaaacccctaaaaacaa aacaaacaca cacacacaca |
| | | | catacacaca cacacacaaa actttgaggcagcgcattgt |
| | | | tttgcatcct tttggcgtga tatccatatg aaattcatgg |
| | | | cttttttttttttgcatat taaagataag acttcctcta ccaccacacc |
| | | | aaatgactac tacacactgctcatttgaga actgtcagct |
| | | | gagtggggca ggcttgagtt ttcatttcat atatctatatgtctataagt |
| | | | atataaatac tatagttata tagataaaga gatacgaatt |
| | | | tctatagactgactttttcc attttttaaa tgttcatgtc acatcctaat |
| | | | agaaagaaat tacttctagtcagtcatcca ggcttacctg |
| | | | cttggtctag aatggatttt tcccggagcc |
| | | | ggaagccaggaggaaactac accacactaa aacattgtct |
| | | | acagctccag atgtttctca ttttaaacaactttccactg |
| | | | acaacgaaag taaagtaaag tattggattt ttttaaaggg |
| | | | aacatgtgaatgaatacaca ggacttatta tatcagagtg |
| | | | agtaatcggt tggttggttg attgattgattgattgatac attcagcttc |
| | | | ctgctgctag caatgccacg atttagattt |
| | | | aatgatgcttcagtggaaat caatcagaag gtattctgac |
| | | | cttgtgaaca tcagaaggta ttttttaactcccaagcagt |
| | | | agcaggacga tgatagggct ggagggctat ggattcccag |
| | | | cccatccctgtgaaggagta ggccactctt taagtgaagg |
| | | | attggatgat tgttcataat acataaagttctctgtaatt acaactaaat |
| | | | tattatgccc tcttctcaca gtcaaaagga actgggggtttggtttttg |
| | | | ttgctttttt agatttattg tcccatgtgg gatgagtttt |
| | | | taaatgccacaagacataat ttaaaataaa taaactttgg |
| | | | gaaaaggtgt aaaacagtag ccccatcaca tttgtgatac |
| | | | tgacaggtat caacccagaa gcccatgaac tgtgtttcca |
| | | | tcctttgcatttctctgcga gtagttccac acaggtttgt aagtaagtaa |
| | | | gaaagaaggc aaattgattcaaatgttaca aaaaaaccct |
| | | | tcttggtgga ttagacaggt taaatatata |
| | | | aacaaacaaacaaaaattgc tcaaaaaaga ggagaaaagc |
| | | | tcaagaggaa aagctaagga ctggtaggaaaaagctttac |
| | | | tctttcatgc cattttattt ctttttgatt tttaaatcat |
| | | | tcattcaatagataccaccg tgtgacctat aattttgcaa atctgttacc |
| | | | tctgacatca agtgtaattagcttttggag agtgggctga |
| | | | catcaagtgt aattagcttt tggagagtgg gttttgtccattattaataa |
| | | | ttaattaatt aacatcaaac acggcttctc atgctatttc |
| | | | tacctcactttggttttggg gtgttcctga taattgtgca cacctgagtt |
| | | | cacagcttca ccacttgtccattgcgttat ttttttttc ctttataatt |
| | | | ctttcttttt ccttcataat tttcaaaagaaaacccaaag ctctaaggta |
| | | | acaaattacc aaattacatg aagatttggt ttttgtcttgcatttttttc |
| | | | ctttatgtga cgctggacct tttctttacc caaggatttt |
| | | | taaaactcagatttaaaaca aggggttact ttacatccta |
| | | | ctaagaagtt taagtaagta agtttcattctaaaatcaga |
| | | | ggtaaataga gtgcataaat aattttgttt taatcttttt |
| | | | gtttttcttttagacacatt agctctggag tgagtctgtc ataatatttg |
| | | | aacaaaaatt gagagctttattgctgcatt ttaagcataa ttaatttgga |
| | | | cattatttcg tgttgtgttc tttataaccaccaagtatta aactgtaaat |
| | | | cataatgtaa ctgaagcata aacatcacat ggcatgttttgtcattgttt |
| | | | tcaggtactg agttcttact tgagtatcat aatatattgt |
| | | | gttttaacaccaacactgta acatttacga attatttttt taaacttcag |
| | | | ttttactgca ttttcacaacatatcagact tcaccaaata tatgccttac |
| | | | tattgtatta tagtactgct ttactgtgtatctcaataaa gcacgcagtt |
| | | | atgttacaaa aaa |

A truncated mini or micro-dystrophin has an amino-terminal actin-binding domain, a dystroglycan-binding domain, and a spectrin-like repeat domain. The spectrin-like repeat domain can include at least four spectrin-like repeats or portions of at least four spectrin-like repeat, and two of the at least four spectrin-like repeats will comprise a neuronal nitric oxide synthase binding domain. The neuronal nitric oxide synthase binding domain is comprised by spectrin-like repeats 16 and 17 relative to the full length polypeptide. The spectrin-like repeat domain can include, for example, four, five, six, seven, eight, or more spectrin-like repeats or portions thereof. The protein encoded by the micro-dystrophin gene can further comprise a hinge domain or a portion thereof. For example, the protein encoded by a mini- or micro-dystrophin gene can include at least a portion of a hinge domain selected from at least one of a Hinge 1 domain, a Hinge 2 domain, a Hinge 3 domain, a Hinge 4 domain, and/or a hinge-like domain (such as the hinge-like domains encoded by the sequences downstream from spectrin-like repeat 15 and within spectrin-like repeat 23.

Duchenne muscular dystrophy (DMD) is a recessively-inherited muscle wasting disorder afflicting approximately 1 in 3500 males. DMD patients carry a mutation in the dystrophin gene, resulting in aberrant or absent expression of the dystrophin protein. DMD patients experience progressive wasting of skeletal muscles and cardiac dysfunction, leading to loss of ambulation and premature death, primarily due to cardiac or respiratory failure. Current available treatments are generally only able to slow the pathology of DMD (see Emery, A. E. H. and Muntoni, F., Duchenne Muscular Dystrophy, Third Edition (Oxford University Press, 2003)). Gene therapy approaches for DMD have been demonstrated in dystrophic animal models by either directly targeting a class of mutations, as with exon skipping, or replacing the mutated gene with viral-vector mediated delivery (see Koo, T. and Wood, M. J. Human Gene Therapy 24, (2013); Benedetti, S., et al., The FEBS Journal 280, 4263-4280, (2013); and Seto, J. T., et al., Current Gene Therapy 12, 139-151 (2012)). Recombinant adeno-associated virus (rAAV) vectors are a potential vehicle for gene therapy, being already tested in clinical trials for both DMD and limb-girdle muscular dystrophies (see Mendell, J. R., et al., The New England Journal of Medicine 363, 1429-1437, (2010); Mendell, J. R., et al., Annals of Neurology 68, 629-638 (2010); and Herson, S., et al., Brain: A Journal of Neurology 135, 483-492, (2012)). Several serotypes of adeno-associated virus (AAV) demonstrate a high degree of tropism for striated muscles (see Seto, J. T., et al., Current Gene Therapy 12, 139-151 (2012)).

Pre-clinical studies designing and testing newer generations of therapeutic constructs for DMD can be confined by the approximately 4.9 kb size of a single-stranded rAAV vector genome (see Dong, B., et al., Molecular Therapy: The Journal of the American Society of Gene Therapy 18, 87-92, (2010) and Wu, Z., et al., Molecular Therapy: The Journal of the American Society of Gene Therapy 18, 80-86, (2010)). Packaging the entire approximately 13.9 kb cDNA of the muscle-specific isoform of dystrophin into a single rAAV capsid cannot be achieved, accordingly, miniaturized, synthetic versions of the muscle-specific isoform of dystrophin cDNA may be used. Although in vivo recombination of two and three rAAV vector genomes has been demonstrated to deliver a mini- or full-length dystrophin coding sequence (see, Odom, G. L., et al., Molecular Therapy: The Journal of the American Society of Gene Therapy 19, 36-45, (2011); Lostal, W., et al., Human Gene Therapy, (2014); and Koo, T., et al., Human Gene Therapy 25, 98-108, (2014)), the efficiency of delivering multiple vectors for reconstituting full-length dystrophin may be suboptimal and can increase the overall dose of viral capsid proteins needed for delivering vectors. However, beneficial rAAV-mediated gene therapy has been achieved using rationally-designed miniature versions of the dystrophin cDNA based in part on mRNA expressed in mild Becker muscular dystrophy patients carrying in-frame deletions within the gene (see Beggs, A. H., et al., American Journal of Human Genetics 49, 54-67 (1991); Koenig, M., et al., American Journal of Human Genetics 45, 498-506 (1989); Goldberg, L. R., et al., Annals of Neurology 44, 971-976, (1998); and England, S. B., et al., Nature 343, 180-182 (1990)). Studies in transgenic and vector treated dystrophic mice expressing various dystrophin truncations have identified various elements of the dystrophin gene that can be present in a functional micro-dystrophin (μDys) (see Harper, S. Q., et al., Nature Medicine 8, 253-261, (2002)).

Exemplary reference sequences for full-length dystrophin DNA, RNA and polypeptide are from SEQ ID NO: 56, SEQ ID NO: 57, dystrophin isoform Dp427m (shown by sequences from NCBI Gene ID: 1756) include NG_012232.1, NM_004006.3 mRNA, CCDS14233.1 CDS (SEQ ID NO: 58), and NP_003997.2 (SEQ ID NO: 59). Additional sequences that can also be used for full-length dystrophin DNA, RNA and polypeptide (NCBI Gene ID: 1756) include, but are not limited to the following: NM_000109.4, NP_000100.3, NM_004009.3, NP_004000.1, NM_004010.3, NP_004001.1, NM_004011.4, NP_004002.3, NM_004012.4, NP_004003.2, NM_004013.3, NP_004004.2, NM_004014.3, NP_004005.2, NM_004015.3, NP_004006.1, NM_004016.3, NP_004007.1, NM_004017.3, NP_004008.1, NM_004018.3, NP_004009.1, NM_004019.3, NP_004010.1, NM_004020.4, NP_004011.3, NM_004021.3, NP_004012.2, NM_004022.3, NP_004013.2, NM_004023.3, NP_004014.2, NC_000023.11, XM_006724469.4, XP_006724532.1, XM_011545467.2, XP_011543769.1, XM_006724473.3, XP_006724536.1, XM_006724474.4, XP_006724537.1, XM_006724475.3, XP_006724538.1, XM_006724470.4, XP_006724533.1, XM_017029328.2, XP_016884817.1, XM_006724468.3, XP_006724531.1, XM_011545468.3, XP_011543770.1, XM_017029330.3, XP_016884819.1, XM_017029329.2, XP_016884818.1, XP 047297845.1, XP_047297845.1, XM_011545469.2, and XP_011543771.1.

The full-length striated muscle isoform of dystrophin plays a role in transmitting contractile force through the sarcolemma and out to the extracellular matrix. In addition to maintaining the mechanical link between the intracellular cytoskeleton and the membrane bound dystrophin glycoprotein complex (DGC), dystrophin can also be a scaffold for signaling proteins (see Ozawa, E. in Myology (ed. Franzini-Armstrong C Engel A) 455-470 (McGraw-Hill, 2004); Winder, S. J. Journal of Muscle Research and Cell Motility 18, 617-629 (1997); and Campbell, K. P. and Kahl, S. D. Nature 338, 259-262, (1989)). The amino-terminal domain of dystrophin can bind to F-actin filaments of the intracellular cytoskeleton (see Way, M., et al., FEBS Letters 301, 243-245 (1992); Hemmings, L., et al., The Journal of Cell Biology 116, 1369-1380 (1992); Fabbrizio, E., et al., Biochemistry 32, 10457-10463 (1993); and Pavalko, F. M. and Otey, C. A. Proceedings of the Society for Experimental Biology and Medicine 205, 282-293 (1994)). The middle, rod domain is the largest and is composed of 24 spectrin-like repeats (SRs) that are flanked and interspersed with at least four hinge sub-domains. The rod domain can give dystrophin elasticity and flexibility for maintaining the integrity of the sarcolemma during muscle contractility (see Winder, S. J. Journal of Muscle Research and Cell Motility 18, 617-629 (1997)). Various SRs provide unique regions that can serve as additional binding sites for the intracellular cytoskeleton, the sarcolemma, as well as members of the DGC (see Rybakova, I. N., et al., The Journal of Cell Biology 135, 661-672 (1996); Warner, L. E., et al., Human Molecular Genetics 11, 1095-1105 (2002); Metzinger, L., et al., Human Molecular Genetics 6, 1185-1191 (1997); Lai, Y., et al., The Journal of Clinical Investigation 119, 624-635, (2009)). In particular, the cysteine-rich domain and the adjacent Hinge 4 region form the β-dystroglycan binding domain (Dg B D) (see Blake, D. J., et al., Physiological Reviews 82, 291-329, (2002); Ishikawa-Sakurai, M., et al., Human Molecular Genetics 13, 693-702, (2004)), while the carboxy-terminal domain is a scaffold for additional DGC components (see Abmayr S, in Molecular Mechanisms of Muscular Dystrophies (ed. Winder, S. J.) 14-34 (Landes Biosciences, 2006)).

Function of dystrophin can be measured using assays that assess specific force and force injury. Examples include, but are not limited to assays that measure force-length relationship, force-velocity relationship, and muscle force generation.

Partially functional micro-dystrophins can improve the dystrophic pathology in striated muscle by protecting the sarcolemma from contraction-induced injury and increasing the capacity to generate force. These parameters can be achieved by binding to F-actin filaments and β-dystroglycan through the amino-terminal domain and the Dg B D (see Harper, S. Q., et al., Nature Medicine 8, 253-261, (2002); Warner, L. E., et al., Human Molecular Genetics 11, 1095-1105 (2002); Cox, G. A., et al., Nature Genetics 8, 333-339, (1994); Greenberg, D. S., et al., Nature Genetics 8, 340-344, (1994); Gardner, K. L., et al., Gene Therapy 13, 744-751, (2006); Corrado, K., et al., The Journal of Cell Biology 134, 873-884 (1996); and Rafael, J. A., et al., The Journal of Cell Biology 134, 93-102 (1996)). Without being bound by any one particular theory, prior studies indicate these two domains must be connected by at least four SRs from the central rod domain, but there are numerous ways in which miniaturized dystrophins containing at least four SRs can be constructed. While some combinations of SRs have been shown to improve the dystrophic pathophysiology, other combinations have not yielded proteins with significant functional capacity (see Harper, S. Q., et al., Nature Medicine 8, 253-261, (2002) and Abmayr S, in Molecular Mechanisms of Muscular Dystrophies (ed. Winder, S. J.) 14-34 (Landes Biosciences, 2006)). Selection of specific SRs in μDys design can restore additional DGC components to the sarcolemma. Neuronal nitric oxide synthase (nNOS) is a signaling protein that can be involved in vasodilation in response to muscle contractile activity (see Stamler, J. S. and Meissner, G. Physiological Reviews 81, 209-237 (2001); Brenman, J. E., et al., Cell 82, 743-752 (1995); Kobayashi, Y. M., et al., Nature 456, 511-515, (2008); and Torelli, S., et al., Neuropathology and Applied Neurobiology 30, 540-545, (2004)), and the presence of SRs 16 and 17 and its interacting partner al syntrophin (Adams 2018 HMG), facilitating proper association of nNOS with the DGC (see 28 Lai, Y. et al., The Journal of Clinical Investigation 119, 624-635, (2009) and Lai, Y., et al., Proceedings of the National Academy of Sciences of the United States of America 110, 525-530, (2013)).

Sequences within spectrin-like repeats 20-24 as well as Hinge 4 can play a role in proper association of dystrophin with microtubules, which can be important for maintaining the intracellular architecture and torque production in skeletal muscle (see Prins, K. W. et al., The Journal of Cell Biology 186, 363-369, (2009) and Belanto, J. J., et al., Proceedings of the National Academy of Sciences of the United States of America 111, 5723-5728, (2014)). Nonetheless, the carboxy-terminal domain and most of the SR domains have been found dispensable without severely compromising the health of striated muscles (see McCabe, E. R., et al., The Journal of Clinical Investigation 83, 95-99, (1989); Crawford, G. E., et al., The Journal of Cell Biology 150, 1399-1410 (2000); and Dunckley, M. G., et al., FEBS Letters 296, 128-134 (1992)).

Several of the best micro-dystrophins tested to date can protect muscles from contraction-induced injury and restore some, but generally not all, of the specific force generating capacity to dystrophic mouse and canine models for DMD (see Seto, J. T., et al., Current Gene Therapy 12, 139-151 (2012) and Wang, Z., et al., Frontiers in Microbiology 2, 201, (2011)). Other micro-dystrophins carrying different combinations of SRs and hinges may function less well in dystrophic muscles, and the reasons for differences in functionality are not clear. However, without being bound by any one particular theory, they may relate to effects on micro-dystrophin elasticity, folding, stability, and the ability to assemble sub-portions of the DGC without steric hindrance. Additional description on dystrophin, mini-dystrophins, and micro-dystrophin can be found in U.S. Patent Publication 2018-0148488 A1, 2020-0095298 A1, and 2020-0031890 A1, incorporated by reference herein in their entireties.

### T Cell Epitopes and T Cell Epitope Prediction

T-cell activity is promoted by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocytes (“CTLs”). Activated T cells produce cytokines, chemokines and other molecules that mediate inflammatory and cytotoxic activity against antigens perceived as non-self, which can include replacement versions of dystrophin. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

Major Histocompatibility Complex (MHC) class I and class II molecules are two primary classes of MHC molecules that present antigens to the immune system. MHC Class I molecules are found on the cell surface of all nucleated cells in the bodies of vertebrates. They also occur on platelets, but not on red blood cells. Their function is to display peptide fragments of proteins from within the cell to cytotoxic T cells; this will trigger an immediate response from the immune system against a particular non-self antigen displayed with the help of the MHC class I protein. These are encoded by 8 genes: HLA-A, HLA-B and HLA-C, the so-called classical HLA genes; and non-classical HLA genes HLA-E, HLA-F, HLA-G, HLA-K, and HLA-L.

MHC Class II molecules are normally only expressed and present antigens on the surface of professional antigen-presenting cells. MHC class II proteins include HLA-DM, HLA-DO, HLA-DP, HLA-DQ and HLA-DR. T cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids.

MHC class I and II epitopes can be predicted by computational means. Example of software that can predict MHC binding- and therefore likely antigenic epitopes-based on peptide sequences can be found on the world wide web at sites including but not limited to services.healthtech.dtu.dk/services/MHCMotifViewer/Home.html, and services.healthtech.dtu.dk/service.php?NetMHCIIpan-4.0, and ROSETTA.

In some embodiments, described herein is a recombinant human dystrophin polypeptide comprising one or more mutations in one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, wherein the recombinant polypeptide retains dystrophin activity.

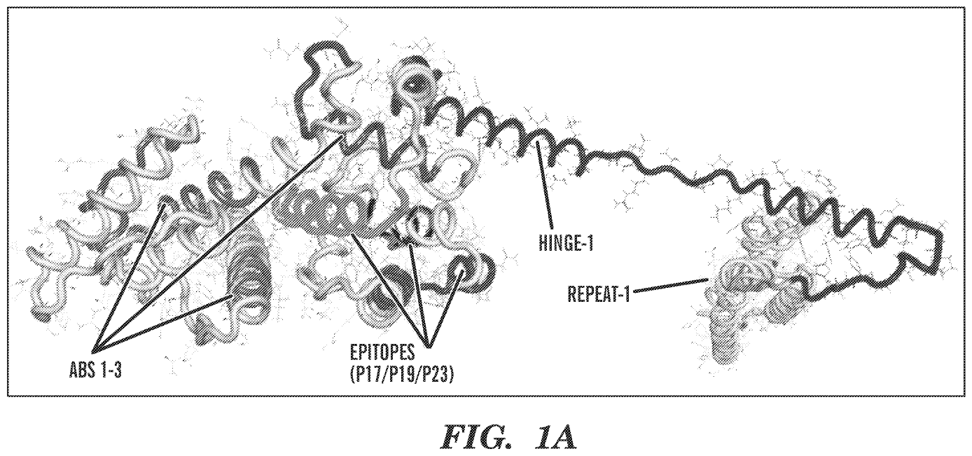

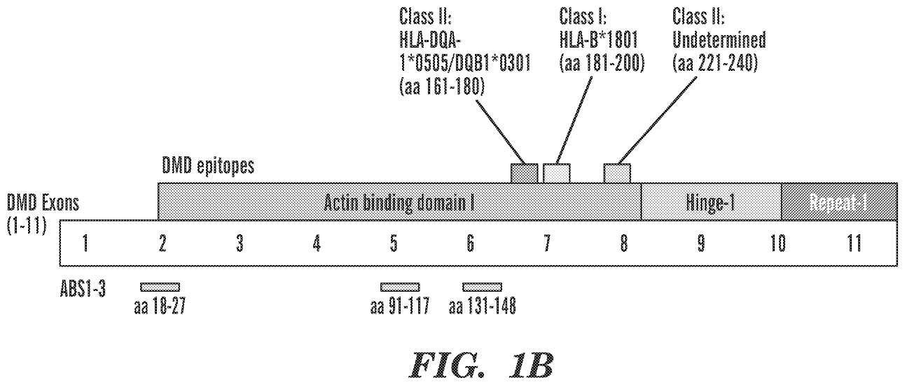

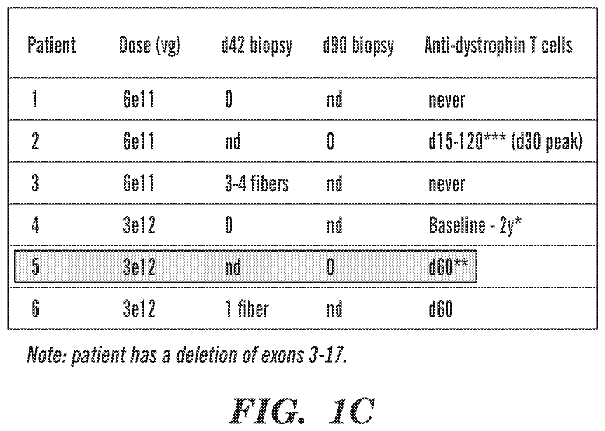

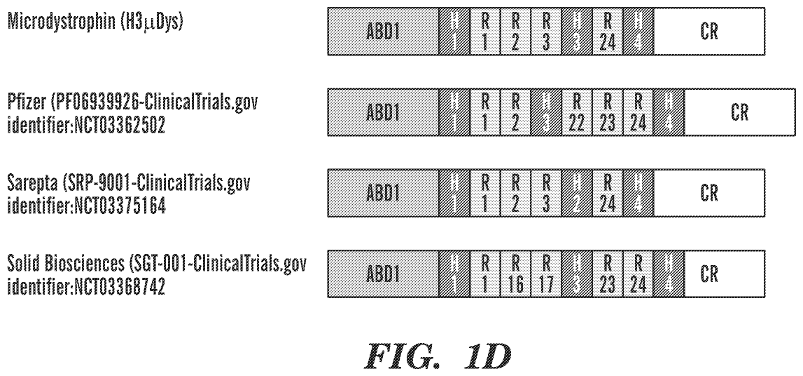

In some embodiments, the polypeptide is a full-length dystrophin polypeptide, a mini-dystrophin polypeptide, or a microdystrophin polypeptide. The one or more mutations described herein can be incorporated into the corresponding Sequence Motif A and/or Sequence Motif B of any full-length dystrophin polypeptide, mini-dystrophin polypeptide, or microdystrophin polypeptide known in the art. Non-limiting examples of microdystrophins that can include the described mutation(s) include: H3μDys (ABD1-H1-R1-R2-R3-H3-R24-H4-CR), the PFIZER microdystrophin (e.g., ABD1-H1-R1-R2-H3-R22-R23-R24-H4-CR), the SAREPTA microdystrophin (e.g., ABD1-H1-R1-R2-R3-H2-R24-H4-CR), or the SOLID BIOSCIENCES microdystrophin (e.g., ABD1-H1-R1-R16-R17-H3-R23-R24-H4-CR) (see e.g., FIG. 1D).

### De-Immunized Dystrophin Polypeptides

As discussed herein above, the instant technology relates to therapeutic human dystrophin polypeptides that have been modified to reduce immune recognition while maintaining dystrophin activity in muscle. Expression of such recombinant dystrophin polypeptides can augment muscle function while avoiding or mitigating immune recognition and attack that would otherwise limit the efficacy of the gene therapy approach.

The modifications to the dystrophin polypeptides are designed to reduce binding of peptides spanning these mutations to MHC-I and MHC-II T-cell receptors. Without such binding, peptides will fail to be presented (by the antigen presenting cell) to the T cell receptor which in turn is necessary to evoke signalling events leading to IFN-gamma production. The reduction in T cell activation effected by a given mutation or set of mutations to known or predicted T cell epitopes can thus be assayed by measuring IFN-gamma production by T cells. Such production can be measured, for example, by an ELISpot assay. The assay employs a capture antibody specific for human IFN-gamma, pre-coated onto a PVDF-backed microplate. If appropriately stimulated cells are within the wells, the immobilized antibody in the immediate vicinity of the secreting cell binds secreted human IFN-gamma. Following wash steps and incubation with a biotinylated detection antibody, alkaline-phosphatase conjugated to streptavidin is added. Unbound enzyme is subsequently removed by washing and a substrate solution (BCIP/NBT) is added. A blue-black colored precipitate forms at the sites of cytokine localization and appears as spots, with each individual spot representing an individual human IFN-gamma secreting cell. The spots can be counted with an automated ELISpot reader system or manually using a stereomicroscope. Thus, IFN-gamma production is the readout for an activated T cell population.

As a demonstration of dystrophin de-immunization, modifications in two sequence motifs, termed Sequence Motif A and Sequence Motif B herein, modify T cell epitopes and blunt T cell activation by the proteins while maintaining dystrophin activity. In some embodiments, the recombinant human dystrophin polypeptide comprises one or more mutations in Sequence Motif A that blunt T cell activity against a T cell epitope in Sequence Motif A. In some other embodiments, the recombinant human dystrophin polypeptide comprises one or more mutations in Sequence Motif B that blunt T cell activity against a T cell epitope in Sequence Motif B. In some other embodiments, the recombinant human dystrophin polypeptide comprises one or more mutations in Sequence Motif A and Sequence Motif B that blunt T cell activity against T cell epitopes in Sequence Motifs A and B.

In some embodiments in any of the aspects is a method of reducing T cell activity against a recombinant human dystrophin polypeptide, the method comprising mutating one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, and wherein the recombinant polypeptide retains dystrophin activity.

In some embodiments, the one or more mutations is/are substitution mutations.

In some embodiments, the mutation in Sequence Motif A comprises amino acid changes at positions 167 and 168. In some other embodiments, the mutation in Sequence Motif A consists of amino acid changes at positions 167 and 168. In some embodiments, the mutation at position 167 is L167T. In some other embodiments, the mutation at position 168 is A168G. In some other embodiments, the mutation at position 167 is L167T, and the mutation at position 168 is A168G.

In some embodiments, the mutation in Sequence Motif A comprises amino acid changes at positions 163, 167 and 168.

In some other embodiments, the mutation in Sequence Motif A consists of amino acid changes at positions 163, 167 and 168. In some embodiments, the mutation at position 163 is W163L. In some other embodiments, the mutation at position 167 is L167T. In some other embodiments, the mutation at position 168 is A168G. In some other embodiments, the mutation at position 163 is W163L, the mutation at position 167 is L167T, and the mutation at position 168 is A168G.

In some embodiments, the mutation in Sequence Motif B comprises amino acid changes at positions 188, 190, 193, 194 and 197. In some other embodiments, the mutation in Sequence Motif B further comprises an amino acid change at position 184.

In some other embodiments, the mutation in Sequence Motif B consists of amino acid changes at positions 188, 190, 193, 194 and 197. In some other embodiments, the mutation in Sequence Motif B consists of amino acid changes at positions 184, 188, 190, 193, 194 and 197.

In some embodiments, the amino acid change at position 184 is N184D. In some embodiments, the amino acid change at position 188 is C188Q, C188K or C188N. In some embodiments, the amino acid change at position 190 is Q190D. In some embodiments, the amino acid change at position 193 is T193K. In some embodiments, the amino acid change at position 194 is Q194Y. In some embodiments, the amino acid change at position 197 is E197Q or E197K.

In some embodiments in any of the aspects is a recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9).

In some other embodiments in any of the aspects is a recombinant human dystrophin polypeptide in which the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).

In some other embodiments in any of the aspects is a recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9), and the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).

In some embodiments, the recombinant human dystrophin polypeptide is a full-length dystrophin polypeptide, a mini-dystrophin polypeptide, or a microdystrophin polypeptide.

Nucleic acid sequences encoding the deimmunized Sequence Motif A and B include are provided herein below.

### Mutagenesis

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

### Delivery of De-Immunized Dystrophins

Gene therapy methods can be used for delivering (e.g., at sustained levels) specific proteins into patients or subjects. These methods allow practitioners to introduce DNA coding for a gene of interest directly into a patient or subject (in vivo gene therapy) or into cells isolated from a patient, a subject, or a donor (ex vivo gene therapy). The introduced DNA then directs the patient's or subject's own cells or grafted cells to produce the desired protein product.

In one aspect, described herein is a nucleic acid encoding a human recombinant dystrophin polypeptide of any one of the described embodiments. In another aspect, described herein is a plurality of nucleic acids, which together encode a human recombinant dystrophin polypeptide of any one of the described embodiments. In one aspect, described herein is a vector comprising a nucleic acid encoding a human recombinant dystrophin polypeptide of any one of the embodiments described herein. In another aspect, described herein is a plurality of vectors each comprising a nucleic acid, which together encode a human recombinant dystrophin polypeptide of any one of the embodiments described herein. For example, in embodiments where the dystrophin gene is too large to be expressed and/or delivered in a single nucleic acid, the dystrophin gene can be divided into at least two nucleic acids and/or vectors, and the expressed polypeptides can be spliced together into a single dystrophin polypeptide, using methods as known in the art, including but not limited to split inteins, see e.g., International Patent Publication WO2023004125A2, the contents of which are incorporated herein by reference in their entirety.

Nucleic acid sequences encoding de-immunized dystrophin polypeptides as described herein can be inserted into any of a range of different vectors for delivery to cells. The sequences can be operably linked to regulatory sequences that interact with cellular expression machinery to drive expression in muscle cells. Most often, expression from a muscle-specific promoter, or promoter/enhancer expression cassette will be preferred, as muscle-specific or muscle-restricted expression not only tends to provide high expression in muscle cells, but helps to further avoid or limit immune sensitization by avoiding expression in non-muscle cells, including professional antigen-presenting cells. Cassettes for driving muscle-specific and/or muscle-restricted expression of transgenes are described, for example, in International Patent Publication WO2022216988A2, filed as PCT/US2022/023915, which is incorporated by reference herein in its entirety.

Vectors for delivery of a nucleic acid construct to cell or tissue can be viral or non-viral. A viral vector includes at least one element of viral origin and has the capacity to be packaged into a viral particle. The viral vector can contain a nucleic acid encoding a polypeptide as described herein in place of non-essential viral genes. The vector and/or particle can be utilized for the purpose of transferring nucleic acids into cells either in vitro or in vivo. Numerous forms of viral vectors are known in the art.

Current viral-mediated gene delivery methods include, but are not limited to, retrovirus, adenovirus, herpes virus, pox virus, and adeno-associated virus (AAV) vectors.

One viral system that has been extensively characterized for gene delivery is adeno-associated virus (AAV). AAV is a parvovirus which belongs to the genus Dependoparvovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions which lends itself to production, storage, and transportation requirements.

The AAV genome is a linear, single-stranded DNA molecule containing 4681 nucleotides. The AAV genome generally comprises an internal non-repeating genome flanked on each end by inverted terminal repeats (ITRs). The ITRs are approximately 145 base pairs (bp) in length. The ITRs have multiple functions, including as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins are expressed from the AAV rep region, Rep78, Rep68, Rep52, and Rep40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus, or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

An AAV vector comprises a vector derived from an adeno-associated virus serotype, including without limitation, AAV1, AAV2, AAV3, AAV4, AAV5, AAV6, AAV7, AAV8, and AAV9 (or AAV9myo derivatives). AAV vectors can have one or more of the AAV wild-type genes deleted in whole or part, e.g., the rep and/or cap genes, but retain functional flanking ITR sequences. Functional ITR sequences are necessary for the rescue, replication, and packaging of the AAV virion. Thus, an AAV vector includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

A recombinant AAV vector or rAAV vector comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery.

Another aspect of the disclosure relates to pharmaceutical compositions comprising nucleotide sequences encoding recombinant dystrophin polypeptides as discussed above.

In various embodiments, the AAV vector may be a serotype 6 AAV (AAV6). Likewise, the rAAV vector can be a serotype 6 rAAV (rAAV6). The AAV vector can be a serotype 8 AAV (AAV8). Likewise, the rAAV vector can be a serotype 8 rAAV (rAAV8). The AAV vector can be a serotype 9 AAV (AAV9). Likewise, the rAAV vector can be a serotype 9 rAAV (rAAV9). The rAAV vector can be comprised of AAV2 genomic inverted terminal repeat (ITR) sequences pseudotyped with capsid proteins derived from AAV serotype 6 (rAAV2/6). Other suitable serotypes of the AAV or rAAV are also within the scope of this disclosure.

In some embodiments, the only modifications to the dystrophin polypeptide are de-immunizing modifications, relative to a wild-type or initial dystrophin polypeptide.

### Pharmaceutical Compositions

In some embodiments of any of the aspects, a pharmaceutical formulation can be administered to a subject comprising a nucleic acid or a vector and a pharmaceutically acceptable carrier. In some other embodiments, a cell can be administered to a subject comprising a recombinant human dystrophin polypeptide, or a nucleic acid or vector encoding such polypeptide.

Treating a subject can comprise delivering an effective amount of a therapeutic composition to a subject (e.g., a patient). An “effective amount” is an amount that can result in a desired physiological change in a subject. While current forms of gene therapy would not generally be administered prophylactically, it is considered that administering a dystrophin gene therapeutic composition or regimen to a subject who has a muscular dystrophy, e.g., DMD, that has not yet progressed, e.g., to cardiac involvement could be prophylactic in terms of preventing the development of cardiac involvement. To this extent and others like it, prophylactic administration is specifically contemplated. In some embodiments, treatment can be directed toward a subject diagnosed with a muscular dystrophy. The muscular dystrophy can be selected from at least one of myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy or other muscular dystrophy or an X-linked dilated cardiomyopathy.

“Therapeutically effective amounts” comprise amounts that provide therapeutic and/or prophylactic treatment. Therapeutically effective amounts need not fully prevent or cure the disease or the condition but can also provide a partial benefit, such as a delay of onset or an alleviation or an improvement of at least one symptom of the disease or the condition.

For administration, effective amounts and therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, a veterinarian, or a researcher, taking into account parameters such as, but not limited to, physical and physiological factors including body weight, severity of condition, type of disease, previous or concurrent therapeutic interventions, idiopathy of the subject, and/or route of administration.

Doses can range from 1×10⁸ vector genomes per kg (vg/kg) to 1×10¹⁵ vg/kg, from 1×10⁹ vg/kg to 1×10¹⁴ vg/kg, from 1×10¹⁹ vg/kg to 1×10¹³ vg/kg, or from 1×10¹¹ vg/kg to 1×10¹² vg/kg. In other non-limiting examples, a dose can comprise about 1×10⁸ vg/kg, about 1×10⁹ vg/kg, about 1×10¹⁹ vg/kg, about 1×10¹¹ vg/kg, about 1×10¹² vg/kg, about 1×10¹³ vg/kg, about 1×10¹⁴ vg/kg, or about 1×10¹⁵ vg/kg. Therapeutically effective amounts can be achieved by administering single or multiple doses during the course of a treatment regimen (i.e., days, weeks, months, etc.).

The formulations described herein can be administered by, without limitation, injection, infusion, perfusion, inhalation, lavage, and/or ingestion. Routes of administration can include, but are not limited to, intravenous, intradermal, intraarterial, intraperitoneal, intralesional, intracranial, intraarticular, intraprostatic, intrapleural, intratracheal, intranasal, intravitreal, intravaginal, intrarectal, topically, intratumoral, intramuscular, intravesicular, intrapericardial, intraumbilical, intraocular, mucosal, oral, subcutaneous, and/or subconjunctival. In other non-limiting examples, administration can be performed by intramuscular injection, intravascular injection, intraperitoneal injection, or any other method suitable for delivery of vector to musculature.

In some embodiments, for injection, formulations can be made as aqueous solutions, such as in buffers including, but not limited to, Hanks' solution, Ringer's solution, and/or physiological saline. The solutions can contain formulatory agents such as suspending, stabilizing, and/or dispersing agents. Alternatively, the formulation can be in lyophilized and/or powder form for constitution with a suitable vehicle control (e.g., sterile pyrogen-free water) before use.

Any formulation disclosed herein can advantageously comprise any other pharmaceutically acceptable carrier or carriers which comprise those that do not produce significantly adverse, allergic, or other untoward reactions that may outweigh the benefit of administration, whether for research, prophylactic, and/or therapeutic treatments. Exemplary pharmaceutically acceptable carriers and formulations are disclosed in Remington's Pharmaceutical Sciences, 18th Ed., Mack Printing Company, 1990, which is incorporated by reference herein for its teachings regarding the same. Moreover, formulations can be prepared to meet sterility, pyrogenicity, general safety, and purity standards as required by the United States FDA's Division of Biological Standards and Quality Control and/or other relevant U.S. and foreign regulatory agencies.

### Measurement/Determination of Efficacy

Methods described herein include methods for increasing dystrophin activity in a cell, as well as treating muscular dystrophy in a subject in need thereof. Upon delivery to a muscle cell or to muscle tissue of a subject, effective improvement in muscle function can be assayed in terms of improved muscular force generation. It can also be useful to monitor subjects for signs of immune activation against the therapeutic dystrophin polypeptide.

Dystrophin activity can be measured using a variety of assays and measurements, which includes, but is not limited to: stability of expression in myogenic cell cultures derived from animal models of Duchenne muscular dystrophy, such as the mdx mouse, the DMD^{mdx} rat, or the GRMD dog models, as measured at intervals up to three weeks after introducing an expression cassette into the myogenic cultures.

Another method of measuring dystrophin activity includes stability of expression in striated muscles from animal models of Duchenne muscular dystrophy, such as the mdx mouse, the DMD^{mdx} rat, or the GRMD dog models, as measured at intervals up to six months after introducing a vector expressing the recombinant protein into the animal model. Such introduction could include injection or infusion of a lentiviral or adeno-associated viral vector, or a plasmid expressing such recombinant proteins. Stable expression is defined as expression levels at least 80%, or at least 90% of that obtained with the corresponding wild type dystrophin protein.

Dystrophin activity is also analyzed using physiological assays to provide an indicator of muscle contractile performance. This is often accomplished by measuring specific force (defined as net force production per unit mass of muscle) generating activity in skeletal muscles from animal models of Duchenne muscular dystrophy, such as the mdx mouse, the DMD^{mdx} rat, or the GRMD dog models. Specific force levels can be measured either in situ or in vitro using isolated skeletal muscles from DMD animal models that have been injected or infused with expression cassettes. An active recombinant dystrophin polypeptide is defined as one that supports maintenance of at least 80%, or at least 90% of the force levels developed with wild type dystrophin polypeptides.

Dystrophin activity is also analyzed by measuring resistance to eccentric contraction (ECC)-induced injury (lengthening contractions) in skeletal muscles from animal models of Duchenne muscular dystrophy, such as the mdx mouse, the DMD^{mdx} rat, or the GRMD dog models. These ECC assays monitor specific force levels either in situ or in vitro in isolated skeletal muscles from DMD animal models that have been injected or infused with expression cassettes, and which are measured before and after a series of eccentric contraction in the muscles. An active recombinant dystrophin polypeptide is defined as one that supports maintenance of at least 80%, or at least 90% of the force levels developed with wild type dystrophin polypeptides after strains of up to 50% of the optimal muscle length for developing force (defined as LO).

Dystrophin activity is also analyzed by measuring ejection fraction, stroke volume and/or extracellular volume fractions in cardiac muscles from animal models of Duchenne muscular dystrophy, such as the mdx mouse, the DMD^{mdx} rat, or the GRMD dog models. These hemodynamic assays monitor the ability of heart muscles to contract and pump blood. Measurements are performed in animal models that display at least a 40% or a 50% reduction in normal hemodynamics, and at intervals at least 3 months after introduction of an expression cassette expressing either wild type or recombinant dystrophin polypeptides. An active recombinant dystrophin polypeptide is defined as one that supports maintenance of at least 80%, or at least 90% of the hemodynamic properties measured in mutant mice expressing a wild type dystrophin polypeptide.

These assays can be adapted or applied to human cell culture assays, e.g., using muscle cell cultures from subjects diagnosed with a muscular dystrophy, or muscle cells differentiated in vitro from induced pluripotent stem cells derived from such a subject creating 3D cultures. Improvements in muscular force generation can be assayed by methods known in the art and/or described in the Examples below. For cardiac muscle, improvements in ejection fraction, stroke volume or other parameters can be measured by methods known in the art, including but not limited to echocardiogram. An increase in muscular force generation, ejection fraction, stroke volume or other parameters of muscular activity by at least 10% following treatment is considered effective therapy, but preferably at least 20%, at least 30%, at least 40%, at least 50%, at least 60%, at least 70%, at least 80% at least 90%, at least 1×, at least 2×, at least 5×, at least 10× or more relative to initial, pre-treatment values. In some embodiments, treatment is considered effective when muscular force generation, ejection fraction, stroke volume, respiratory function, or other parameter is restored or established to within a certain proportion of a normal or standard value, e.g., at least 50%, at least 60%, at least 70%, at least 80%, at least 90%, at least 95% or more relative to a standard, non-diseased value.

In certain embodiments, a pharmaceutical composition as described herein can reduce, or be configured to reduce, a pathological effect or symptom of the muscular dystrophy. Such pathological effect or symptom of the muscular dystrophy may be selected from at least one of muscle pain, muscle weakness, muscle fatigue, muscle atrophy, fibrosis, inflammation, increase in average myofiber diameter in skeletal muscle, cardiomyopathy, reduced 6-minute walk test time, loss of ambulation, cardiac pump failure, and/or one or more other suitable pathological effects or symptoms.

The methods of treating a subject having muscular dystrophy can further comprise identifying a subject having muscular dystrophy. In some embodiments, the subject can be a mammal. In certain embodiments, the subject can be a human.

In certain embodiments, the pharmaceutical composition can reduce, or be configured to reduce, a risk of developing a pathological effect or symptom of a muscular dystrophy.

In certain embodiments, the enhancers or promoters may express, or be configured to express, a pharmaceutical composition comprising a peptide. In various embodiments, the enhancers or promoters may express, or be configured to express, the peptide in developing, injured, and/or diseased muscle (i.e., muscle that may be undergoing regeneration).

As discussed above, the enhancers and/or promoters may be operatively linked to a pharmaceutical composition, i.e., for enhancing expression and/or targeting of the pharmaceutical composition. Additionally, the pharmaceutical composition may be operatively linked to one or enhancers and/or promoters. In some embodiments, expression of the pharmaceutical compositions disclosed herein may assist in regenerating skeletal muscle.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

The term “dystrophin polypeptide” as used herein encompasses full length, mini- and micro-dystrophin proteins that provide a link between the actin-based cytoskeleton of a muscle fiber and the extracellular matrix. Dystrophin structure and structure/function correlations are discussed further herein below. However, a “dystrophin polypeptide” will include, at a minimum, an amino-terminal actin-binding domain, a dystroglycan-binding domain, and a domain comprising a plurality of spectrin-like repeats that includes a neuronal nitric oxide synthase binding domain. Truncated versions of dystrophin that retain significant activity in muscle function have been described. These are roughly divided into two classes referred to as “mini-dystrophins” and “micro-dystrophins” (or “microdystrophins”) which differ primarily in size of the protein and encoding gene sequence—there is not necessarily a hard cutoff, but a mini-dystrophin gene is about 6 to 8 kb in size, and a microdystrophin gene is about 3.5 to 4 kb in size. Full length dystrophin includes four major domains: the N-terminal; rod; cystine-rich and C-terminal domains. The rod domain includes 24 spectrin-like repeats and 4 hinges. Mini-dystrophins delete portions of the rod domain that are not required for function, and micro-dystrophins delete not only portions of the rod domain but also portions of the C-terminal domain.

The term “retains dystrophin activity” as used herein, refers to the link between the actin-based cytoskeleton of the muscle fiber and the extracellular matrix. A dystrophin polypeptide including one or more modifications to a T cell epitope therein as described herein “retains dystrophin activity” if a muscle cell or muscle tissue expressing such dystrophin has at least 30% of the muscle activity of a muscle expressing full length wild-type dystrophin. In various embodiments, a dystrophin polypeptide that retains dystrophin activity has at least 40%, at least 50%, at least 60%, at least 70%, at least 80%, at least 90%, at least 95%, or more of the muscle activity of wild-type dystrophin. In this context, dystrophin activity can be examined using assays that measure force-length relationship, force-velocity relationship, and muscle force generation, among others. Methods for measuring dystrophin activity are described further herein below.

The term “blunts T cell activity against” or “reduces T cell activity against” as used herein is defined as a reduction, as that term is defined herein, in T-cell activity against a dystrophin polypeptide modified in one or more T cell epitopes relative to that dystrophin polypeptide prior to or lacking such modifications. Reduction in T cell recognition/activation can be measured using an assay (e.g., an ELISPOT assay) where T-cell recognition or activation against dystrophin polypeptide decreases by at least 10% as compared to a reference level (e.g. T-cell activity against wild-type dystrophin or a non-T cell-epitope modified dystrophin polypeptide as described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more.

As used herein, a “de-immunized” polypeptide has had one or more predicted or demonstrated MHC Class I or MHC Class II epitopes modified relative to a wild-type or starting polypeptide in a manner that blunts T cell activity against the polypeptide as that term is defined herein. While it can be preferable to modify a plurality of such epitopes when they occur, a de-immunized polypeptide need not have all known or predicted MHC Class I or MHC Class II epitopes modified. A de-immunized polypeptide as described herein will retain at least 40%, at least 50%, at least 60%, at least 70%, at least 80%, at least 90%, at least 95%, or more of the activity of the wild-type or starting polypeptide, e.g., dystrophin polypeptide activity.

The terms “increased” or “increase” are all used herein to mean an increase by a statically significant amount. In some embodiments, the terms “increased” or “increase” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10%-100% as compared to a reference level (e.g. the absence of an isolated nucleic acid molecule, polypeptide, vector, composition, or pharmaceutical composition described herein), or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The terms “decrease”, “reduced”, “reduction”, “blunt”, “blunted”, “blunts”, or “inhibit” are all used herein to mean a decrease by a statistically significant amount. In some embodiments, “blunt,” “reduce”, “reduction” or “decrease” or “inhibit” typically means a decrease by at least 10% as compared to a reference level (e.g. the absence of an isolated nucleic acid molecule, polypeptide, vector, composition, or pharmaceutical composition described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction”, “blunt” or “inhibition” does not encompass a complete inhibition or reduction as compared to a reference level. “Complete inhibition” is a 100% inhibition as compared to a reference level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that provide animal models of disease e.g., cardiac disease or disorder, such as muscular dystrophy. A subject can be male or female.

A subject can be one who has been previously diagnosed with or identified as suffering from or having a condition in need of treatment (e.g., muscular dystrophy) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition (e.g., muscular dystrophy), diagnosed as having that condition, or at risk of developing that condition. Risk factors for muscular dystrophy include, but are not limited to gender and family history of muscular dystrophy.

Replacement of one or more amino acids (e.g., 1 amino acid, 2 amino acids, 3 amino acids, 4 amino acids, 5 amino acids, 6 amino acids, 7 amino acids, 8 amino acids, 9 amino acids, or 10 amino acids, or alternatively, 10 or fewer amino acids, 9 or fewer amino acids, 8 or fewer amino acids, 7 or fewer amino acids, 6 or fewer amino acids, 5 or fewer amino acids, 4 or fewer amino acids, 3 or fewer amino acids, or two or fewer amino acids) in a T cell epitope can abrogate or blunt the efficiency of T cell recognition at that site. Where it is important to maintain the function of the mutant polypeptide, i.e., link between the actin-based cytoskeleton of the muscle fiber and the extracellular matrix, it can be beneficial to modify a site or sites via conservative amino acid substitution(s). In a conservative substitution, a given amino acid can be replaced by a residue having similar physicochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any of the assays described herein to confirm that dystrophin activity, e.g., functional link between the actin-based cytoskeleton of the muscle fiber and the extracellular matrix, is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Alterations of the native amino acid sequence of a dystrophin polypeptide can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+ (% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined within the description of the various aspects and embodiments of the technology.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A recombinant human dystrophin polypeptide comprising one or more mutations in one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, wherein the recombinant polypeptide retains dystrophin activity.
2. The recombinant human dystrophin polypeptide of paragraph 1, wherein the polypeptide is a full-length dystrophin polypeptide, a mini-dystrophin polypeptide, or a microdystrophin polypeptide.
3. The recombinant human dystrophin polypeptide of paragraph 1 or paragraph 2, which comprises one or more mutations in Sequence Motif A that blunt T cell activity against a T cell epitope in Sequence Motif A.
4. The recombinant human dystrophin polypeptide of paragraph 1 or paragraph 2, which comprises one or more mutations in Sequence Motif B that blunt T cell activity against a T cell epitope in Sequence Motif B.
5. The recombinant human dystrophin polypeptide of paragraph 1 or paragraph 2, which comprises one or more mutations in Sequence Motif A and Sequence Motif B that blunt T cell activity against T cell epitopes in Sequence Motifs A and B.
6. The recombinant human dystrophin polypeptide of any one of paragraphs 1-5, wherein the one or more mutations is/are substitution mutations.
7. The recombinant human dystrophin polypeptide of any one of paragraphs 1-6, wherein the mutation in Sequence Motif A comprises amino acid changes at positions 167 and 168.
8. The recombinant human dystrophin polypeptide of paragraph 7, wherein the mutation in Sequence Motif A consists of amino acid changes at positions 167 and 168.
9. The recombinant human dystrophin polypeptide of paragraph 7 or 8, wherein the mutation at position 167 is L167T.
10. The recombinant human dystrophin polypeptide of paragraph 7 or 8, wherein the mutation at position 168 is A168G.
11. The recombinant human dystrophin polypeptide of paragraph 7 or 8, wherein the mutation at position 167 is L167T, and the mutation at position 168 is A168G.
12. The recombinant human dystrophin polypeptide of any one of paragraphs 1-6, wherein the mutation in Sequence Motif A comprises amino acid changes at positions 163, 167 and 168.
13. The recombinant human dystrophin polypeptide of any one of paragraphs 1-6, wherein the mutation in Sequence Motif A consists of amino acid changes at positions 163, 167 and 168.
14. The recombinant human dystrophin polypeptide of paragraph 12 or 13, wherein the mutation at position 163 is W163L.
15. The recombinant human dystrophin polypeptide of paragraph 12 or 13, wherein the mutation at position 167 is L167T.
16. The recombinant human dystrophin polypeptide of paragraph 12 or 13, wherein the mutation at position 168 is A168G.
17. The recombinant human dystrophin polypeptide of paragraph 12 or 13, wherein the mutation at position 163 is W163L, the mutation at position 167 is L167T, and the mutation at position 168 is A168G.
18. The recombinant human dystrophin polypeptide of any one of paragraphs 1-17, wherein the mutation in Sequence Motif B comprises amino acid changes at positions 188, 190, 193, 194 and 197.
19. The recombinant human dystrophin polypeptide of paragraph 18, further comprising an amino acid change at position 184.
20. The recombinant human dystrophin polypeptide of any one of paragraphs 1-17, wherein the mutation in Sequence Motif B consists of amino acid changes at positions 188, 190, 193, 194 and 197.
21. The recombinant human dystrophin polypeptide of any one of paragraphs 1-17, wherein the mutation in Sequence Motif B consists of amino acid changes at positions 184, 188, 190, 193, 194 and 197.
22. The recombinant human dystrophin polypeptide of paragraph 19 or 21, wherein the amino acid change at position 184 is N184D.
23. The recombinant human dystrophin polypeptide of any one of paragraphs 18-22, wherein the amino acid change at position 188 is C188Q, C188K or C188N.
24. The recombinant human dystrophin polypeptide of any one of paragraphs 18-23, wherein the amino acid change at position 190 is Q190D.
25. The recombinant human dystrophin polypeptide of any one of paragraphs 18-24, wherein the amino acid change at position 193 is T193K.
26. The recombinant human dystrophin polypeptide of any one of paragraphs 18-25, wherein the amino acid change at position 194 is Q194Y.
27. The recombinant human dystrophin polypeptide of any one of paragraphs 18-26, wherein the amino acid change at position 197 is E197Q or E197K.
28. A recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9).
29. A recombinant human dystrophin polypeptide in which the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).
30. A recombinant human dystrophin polypeptide in which the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9), and the sequence motif 181-FDWNSVVCQQSATQRLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLQHAF-200 (SEQ ID NO: 19).
31. The recombinant dystrophin polypeptide of paragraph 29 or 30, wherein the dystrophin polypeptide retains dystrophin activity.
32. The recombinant dystrophin polypeptide of paragraph 29 or 30, wherein the dystrophin polypeptide exhibits blunted T cell activity against the polypeptide relative to wild-type dystrophin.
33. A method of reducing T cell activity against a recombinant human dystrophin polypeptide, the method comprising mutating one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, and wherein the recombinant polypeptide retains dystrophin activity.
34. The method of paragraph 33, wherein the recombinant human dystrophin polypeptide is a full-length dystrophin polypeptide, a mini-dystrophin polypeptide, or a microdystrophin polypeptide.
35. A nucleic acid encoding a human recombinant dystrophin polypeptide of any one of paragraphs 1-32.
36. A vector comprising a nucleic acid of paragraph 35.
37. The vector of paragraph 36, wherein the vector is a plasmid or a viral vector.
38. The viral vector of paragraph 37, wherein the vector is a lentiviral vector, foamy viral vector, or an adeno-associated virus (AAV) vector.
39. A pharmaceutical formulation comprising a nucleic acid of paragraph 35 or a vector of any of paragraphs 36-38 and a pharmaceutically acceptable carrier.
40. A cell comprising a recombinant human dystrophin polypeptide of any one of paragraphs 1-33, or a nucleic acid or vector encoding such polypeptide.
41. A method of increasing dystrophin activity in a cell, the method comprising introducing a nucleic acid of paragraph 35 or a vector of any one of paragraphs 36-38 to a cell.
42. The method of paragraph 41, wherein the cell is a muscle cell.
43. A method of treating a muscular dystrophy in a subject in need thereof, the method comprising introducing a nucleic acid of paragraph 35 or a vector of paragraph 36 or 37 to a muscle cell in the subject.
44. The method of paragraph 43, wherein the muscle cell is a skeletal muscle cell or a cardiac muscle cell
45. The method of paragraph 43 or 44, wherein the muscular dystrophy is myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, or an X-linked dilated cardiomyopathy.
46. A method of treating a muscular dystrophy in a subject in need thereof, the metho comprising administering a pharmaceutical formulation of paragraph 39 to the subject, whereby a nucleic acid encoding a recombinant human dystrophin polypeptide is introduced to a muscle cell of the subject.
47. The method of paragraph 46, wherein the muscular dystrophy is myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, or an X-linked dilated cardiomyopathy.

The technology described herein is further illustrated by the following examples which in no way should be construed as being further limiting.

### EXAMPLES

### Example 1: Dystrophin Deimmunization Prevents Human T-Cell Activation and Restores Muscle Function in Mdx⁴cv Mice

Duchenne muscular dystrophy (DMD) and the milder Becker M D are due to mutations in the dystrophin gene, and represent the most common muscular dystrophies. The primary function of dystrophin within muscle tissue is to link the cellular cytoskeleton to the extracellular matrix (ECM) via the dystrophin-glycoprotein complex (DGC). This linkage transduces contractile forces to the ECM to protect skeletal muscle from contraction-induced injury. Ultimately, the absence of dystrophin results in a drastic reduction of all of the DGC components, further contributing to membrane destabilization, membrane permeability defects, myofiber degeneration accompanied by gradual replacement with fibrotic connective, and adipose tissue. Physical symptoms present themselves in boys between ages 2 and 3 or may be evident in early infancy. There is currently no cure for DMD, and patients typically die in early adulthood due to progressive muscle wasting followed by cardiac and respiratory muscle failure. Indeed, large genomic deletions found in some mildly affected BMD patients have catalyzed efforts to make functional micro-dystrophins (μDys) that retain the N-terminal actin binding domain and the cysteine-rich (CR) beta-dystroglycan-binding domain, enabling reassembly of the DGC (FIG. 1A-1E). Various miniaturized dystrophins have been subjected to testing in animal models of DMD, within strains of mdx mice, cxmd dogs and recently mdx rats. The vast majority of studies continue to be in mdx mice, where muscle pathology is milder than in humans, with the exception of diaphragm, but the dystrophic phenotype also worsens with increasing age.

Recombinant adeno-associated viral vectors (rAAV) are attractive, as they transduce numerous cell types and support stable, long-term gene expression with minimal induction of an immune response in mice. Recently, several myotrophic rAAV serotypes have been isolated that display ~10-fold improved myocyte transduction in vivo relative to the parental AAV9 serotype, while also limiting exposure to the liver, potentially reducing liver toxicity-together features that permit the use of lower vector doses while reducing complement activation and thrombocytopenia. While tissue tropism is influenced by AAV serotype, muscle-restricted gene expression can be achieved by using muscle-specific regulatory cassettes (MSRCs), driving mRNA transcription in the appropriate cells or tissue. This is no small task, as muscle genes are regulated by combinatorial associations amongst a population of transcription factors bound to promoters or enhancers and various protein complexes. The expression of muscle specific genes also fluctuates between alternate anatomical muscles and in metabolic slow- or fast-twitch myofiber types, implying alternate mechanisms of regulation in reaction to a range of physiological cues, in addition to the ongoing proinflammatory environment of dystrophic muscles. Combining rAAV delivery with highly active and restrictive MSRCs provides further relative increases in dystrophin production and severely limits expression in non-muscle cells such as antigen presenting cells (APCs). However, not all MSRC's are created equal toward restricted expression or leakiness in antigen-presenting cells. For over a decade there has been serious concern by numerous investigators that DMD patients, particularly those with large intragenic deletions, that vector-delivered dystrophin proteins could be perceived as non-self by the immune system. This, in turn, can lead to diminishing returns on therapeutic efficacy or at worse serious adverse events (SAE's), complicating future treatment options.

Recent reports demonstrate that 5 patients in the Pfizer and SAREPTA AAV-μDys clinical trials developed SAE's and elicited an immune response against μDys. These studies used pseudotyped vectors based on AAV9 or AAVrh74 together with promoter/enhancer elements that were not completely muscle-specific. SOLID BIOSCIENCES has not observed immunity against μDys that rise to the level of SAE's, and while they have not treated as many patients (or for as long of a time period) as the other groups, their vector (AAV9) incorporates a MSRC (CK8e) which has been utilized here for the current protein redesign strategies.

The focus of the epitope redesign studies has centered on a previous clinical trial (Clinicaltrials.gov number, NCT00428935) where the ‘patient 5’ immune system presented with a T-cell response targeting 3 epitopes, previously identified as P17, P19, and P23. It should be noted that the ‘patient 5’ DMD mutation encompassed a deletion of exons 3-17, strikingly similar to deletions found in the recent 5 patients that developed SAEs. The immune cell reactivity was monitored by interferon-gamma (IFNγ) ELISpot assays for quantification of dystrophin peptide responsive T-cells. One exon-7 epitope was recognized by HLA-B*1801-restricted CD8+ T cells, whereas the additional epitope in exon 6 was recognized by CD4+ T cells. The clinical trial outcome pointed to the need to blunt T-cell immunity toward dystrophin epitopes in the design of future trials. Alternatively, MSRC (CK8m) can be used for more extensive expression in the heart.

To address these immunological concerns, several immunological and protein design tools were incorporated. Central to the approach, immunologists have developed computational software for predicting epitopes bound by human MHC I and II, and have identified positive scoring epitopes (strong binders) vs. negative scoring peptides (weak binders) to evaluate vaccine efficacy in silico. Here, immune epitope databases are combined with ROSETTA, an all-atom computational protein structure design tool. To approximate the energy functions within or between macromolecules, ROSETTA incorporates a physics-based force field where folding occurs at the lowest energy state. Overall, ROSETTA takes into account the numerous forces driving the inherent structure of proteins, where highly credible models have been experimentally validated in numerous systems. ROSETTA has been successfully applied to many protein engineering tasks, including epitope modification.

With the identification of dystrophin-specific epitopes in hand, the epitopes were redesigned to be non-immunogenic (“deimmunized”) or severely diminished toward MHC antigen uptake and presentation to the TCR, which lead to signaling cascades and immune activation against the therapeutic dystrophin. Here, the ability to deimmunize individual epitopes using semi-matched T-cells derived from patient-5 was demonstrated. The epitopes from exon-6 and exon-7 reside within 25 amino acids of one another, each in close proximity to a functionally essential actin-binding site. Strategies for the successful redesign of dystrophin-specific epitopes to achieve these ends are described, and comparative data from in vitro and in vivo studies are presented. As an in vitro screening tool, clonally derived mdx myotube cultures evaluated for expression post-differentiation proved valuable toward predicting in vivo physiological outcomes. The leading dual-epitope (exon 6-7) redesigned vectors showed restoration of cardiac left ventricular ejection fraction in aged mdx^{4cv} mice, as determined by cardiac magnetic resonance imaging (cMRI).

### Results

### Redesign and Deimmunization of an Exon-6 (P17) μDys MHC Class II Epitope

A computational deimmunization redesign protocol was utilized with modifications and a method centrally based on atomic data, which removes MHC epitopes by mutating key anchor residues within the peptide sequence, resulting in destabilization of the MHC: peptide interaction, negating associated downstream signaling cascades with subsequent T cell activation. This protocol also increases human sequence content, incorporating updates as they become available as part of the mutagenic outcome to further deter MHC epitope presentation. Additionally, on the other side of destabilizing mutagenic effects, there are also stabilizing mutations that can confer increased stability. In the case of dystrophin (exons 6-8) a primary objective is to maintain avidity of the interaction of the ABD of dystrophin with actin which is critical for maintaining contractile performance of the muscle cell or myofiber. Redesign within this region is further complicated by epitope proximity to the ABS2 within the ABD (FIG. 1A), mutations of which have been shown to highly blunt biological activity of dystrophin.

### Evaluation of P17 Redesign Candidates for Pathophysiological Improvements in Mdx⁴cv Mice

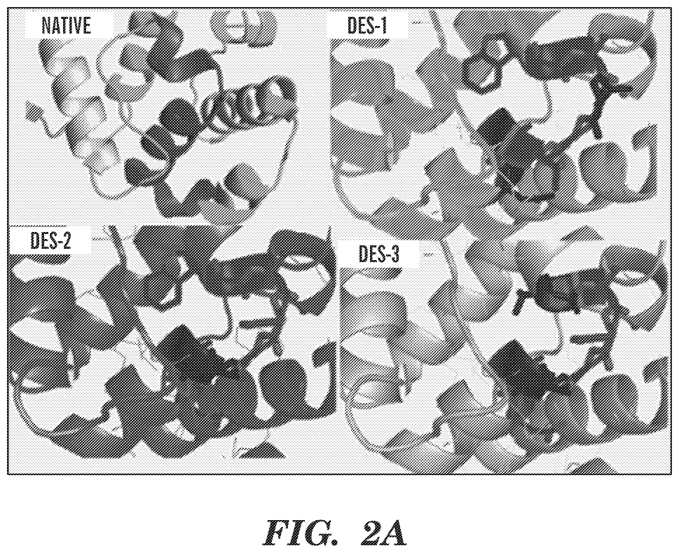

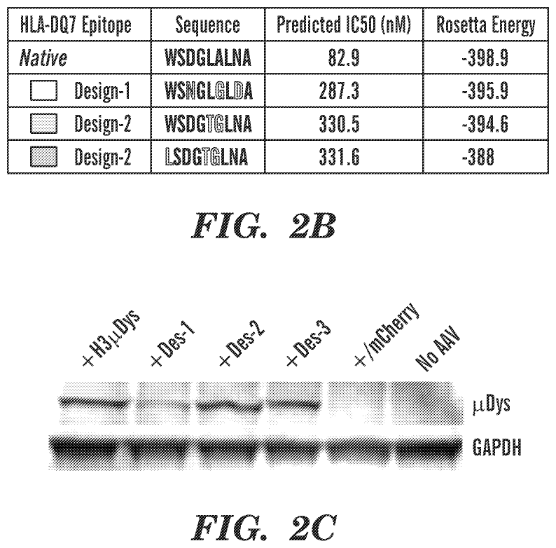

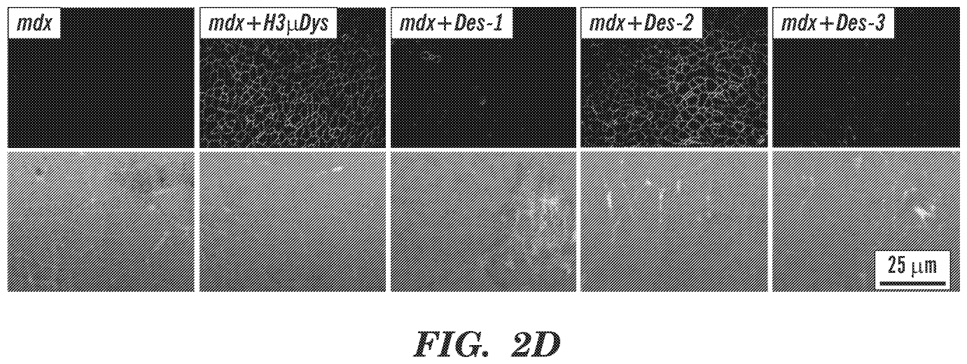

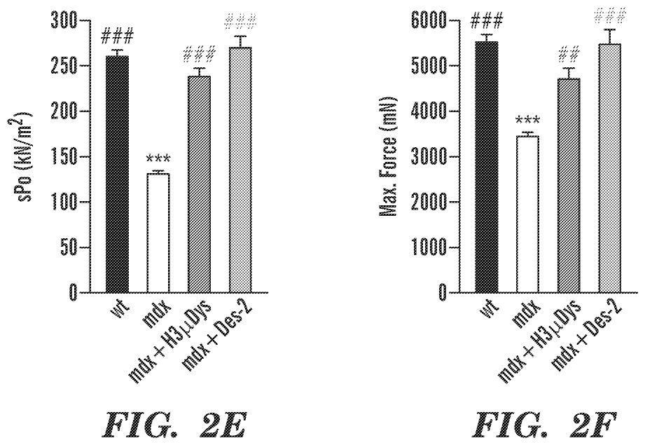

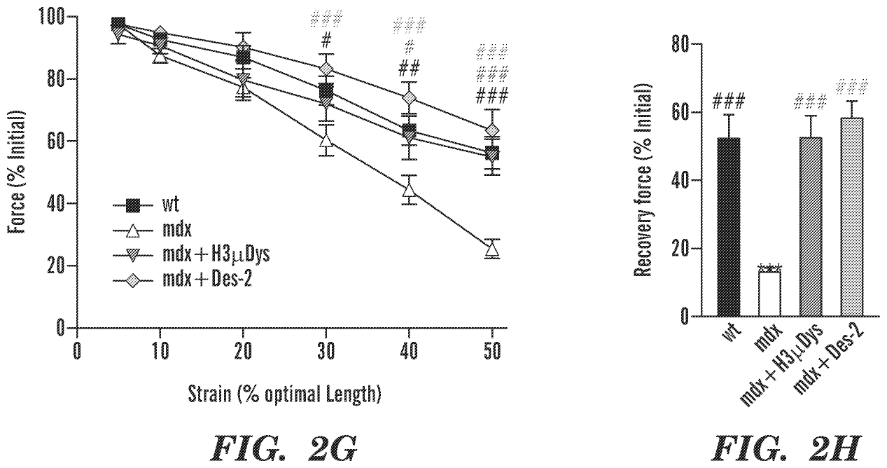

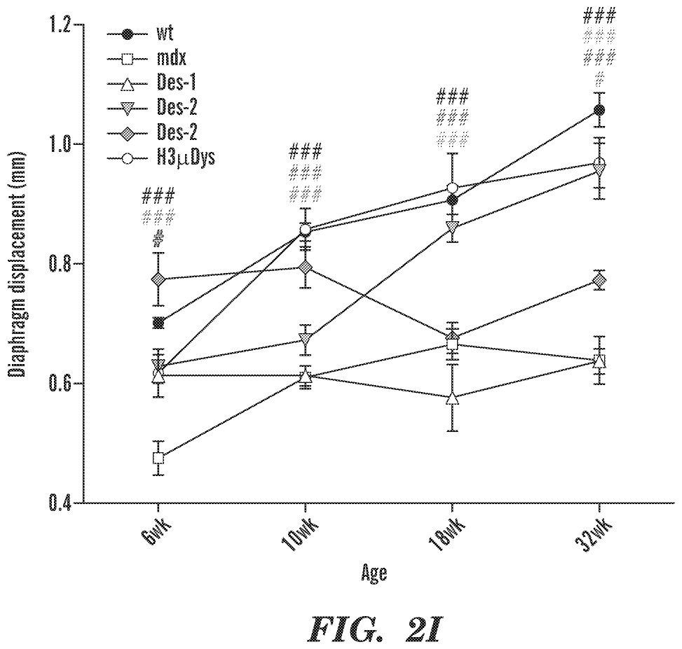

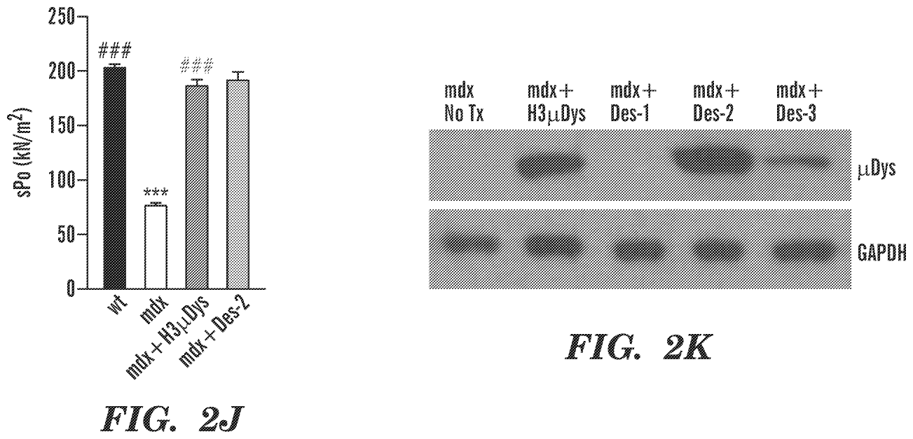

Structural prediction and human MHC II epitope prediction software programs were applied to the native μDys sequence and identified the same peptide (163-WSDGLALNAL-172, SEQ ID NO: 1) that had been detected as a strong epitope for MHC II binding of patient 5.37 ROSETTA all-atom redesign methodology was used to deimmunize this epitope in 3 ways (Des-1,-2,-3; FIG. 2A-2K) while retaining overall structure. Within the epitope core, 3 residue substitutions were applied for Des-1 (163-WSNGLGLDAL-172, SEQ ID NO: 7), 2 residue substitutions for Des-2 (163-WSDGTGLNAL-172, SEQ ID NO: 8) and 3 residue substitutions (163-LSDGTGLNAL-172, SEQ ID NO: 9) for Des-3. Comparatively, ROSETTA energy units were within 1% of one another (FIG. 2A). As such, these three design variants were synthesized in the context of μDys, cloned into rAAV-CK8e and tested in mdx mouse muscles via vector transduction in vitro and in vivo. A direct comparison was made to a standard 4-repeat μDys (FIG. 1D). The Des-2 redesign candidate performed as well as native μDys, as judged by immunostaining (FIG. 2C), in situ physiology (FIG. 2D-2G), western immunoblotting (FIG. 2J-2K). To test whether re-engineered μDys proteins can be screened with a rapid in vitro assay, mdx myogenic cultures were transduced with the three modified μDys constructs Des-1, 2 and 3 as well as native μDys via rAAV-CK8e delivery. Cultures were harvested at 96 h and assayed by western blot for μDys expression. Des-2 and -3 performed similarly to H3μDys, whereas Des-1 levels were ~3-fold lower. Normalized expression levels were: H3μDys=1.0, Des-1=0.33, Des-2=0.95 and Des-3=0.92. To assess physiological function of Des-2 (P17 epitope “best redesign”) relative to parental native μDys in mdx^{4cv} mice, a sonography assay was used to measure diaphragm displacement during respiratory cycles (FIG. 2H). Diaphragm contraction was measured from the relaxation baseline to the maximum contraction peak during 5 respiratory cycles. No significant difference was found in wt and mdx^{4cv} mice treated with either rAAV6-CK8-Des-2 or native rAAV6-CK8-μDys control at 18 and 32 wks. Des-3 performed less well as suggested by in vivo immunostaining and western blot but was correctly localized on the sarcolemma (note H&E staining and representation of peripheral nuclei). In contrast, Des-1 was undetectable in vivo, (and dramatically reduce in vitro by western analysis) suggesting instability/degradation. Diaphragm specific force production was also assayed, yielding 205.9±7.5 (wt, N=5); 78.4±5.6 (mdx, N=5); 185.9±12.1 (mdx+H3μDys, N=4) and 191.2±13.9 kN/m2 (mdx+Des-2, N=4) (FIG. 2I). This demonstrates that epitope prediction and redesign algorithms yield highly functional deimmunized human μDys. All bars in the graphs represent mean±S.D. *P,0.05, **P,0.01 and ***P,0.001 compared to wild-type; #P,0.05, ##P,0.01 and ###P,0.001 compared to mdx^{4cv}.

### EliSpot Assays of P17 Epitope Redesigns

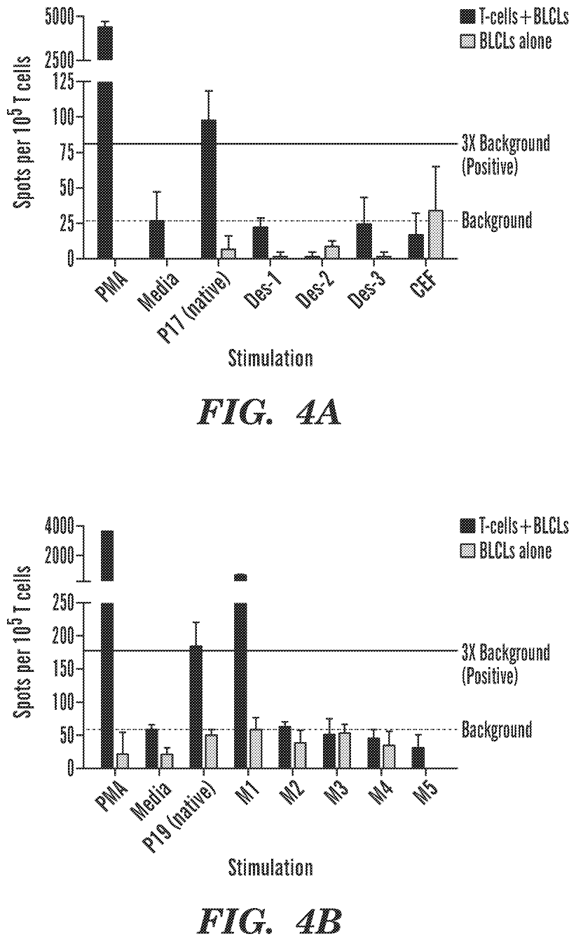

In order to evaluate deimmunization of the exon 6 epitope (P17), interferon gamma EliSpot assays were performed with the expanded patient T cells against the native and redesigned versions of P17 (FIG. 4A). In summary, all three redesigns ablated the IFNγ T cell response where cytokine production was found to be comparable to background levels (~25 spot forming units/10,000 cells). The native epitope recapitulated the previous report of activity showing an ~3-fold signal increase above background (or ~75 spot forming units/10,000 cells).

### Redesign of an Exon-7 (P19) μDys MHC Class I Epitope

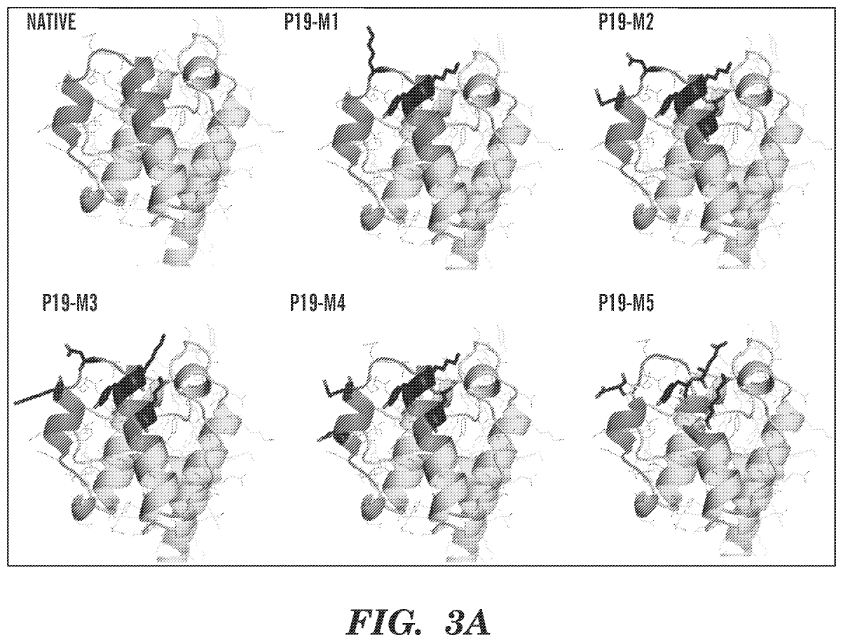

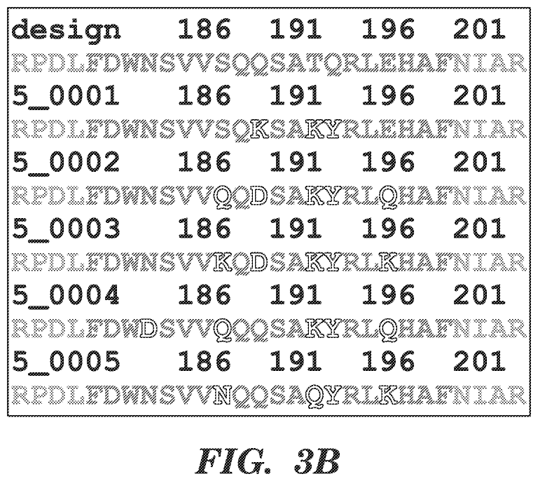

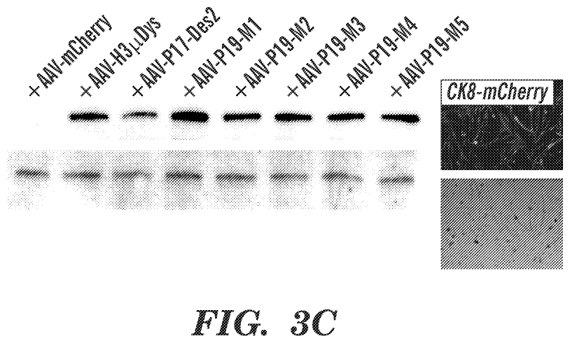

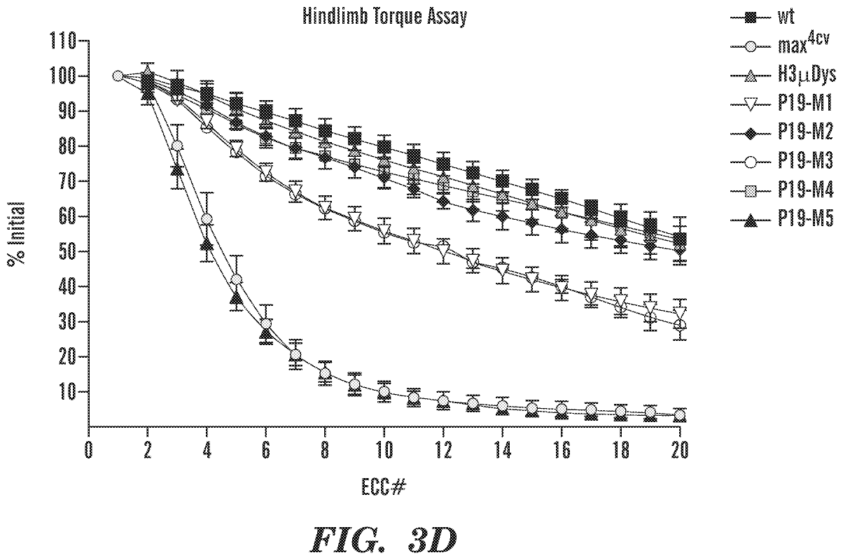

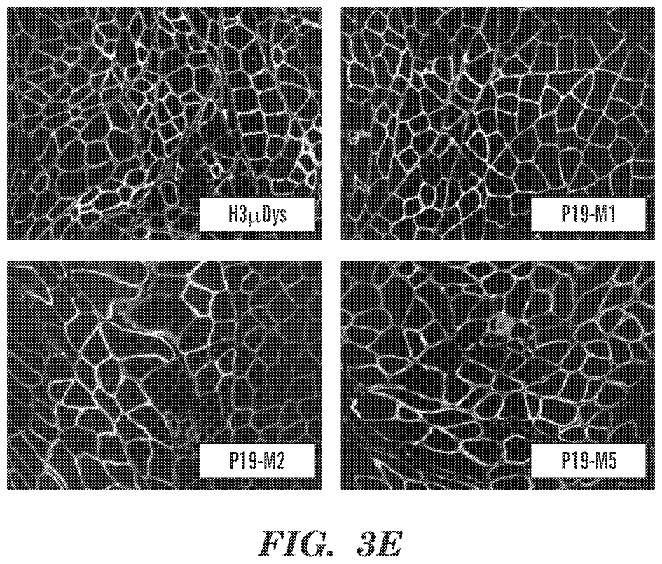

ROSETTA all-atom deimmunization and redesign protocol was repeated for the P19 epitope, again with the objective of engineering subtle changes within the epitope to maintain μDys stability and functionality while preventing MHC binding and subsequent peptide presentation to TCRs and by extension preventing downstream signaling cascades toward the promotion of a specific immune response against dystrophin. The exon-7 epitope was previously determined to be recognized by HLA-B*1801-restricted CD8+ T cells. 5 redesigns were engineered (M1-M5) for the P19 epitope (181-FDWNSVVCQQSATQRLEHAF-200, SEQ ID NO: 2). To summarize, 3 residue substitutions were introduced for M1 (181-FDWNSVVCQKSAKYRLEHAF-200, SEQ ID NO: 16), 5 substitutions for M2 (181-FDWNSVVQQDSAKYRLQHAF-200, SEQ ID NO: 17), 5 substitutions for M3 (181-FDWNSVVKQDSAKYRLKHAF-200, SEQ ID NO: 18), 5 substitutions for M4 (181-FDWDSVVQQQSAKYRLQHAF-200, SEQ ID NO: 19), and 4 substitutions for M5 (181-FDWNSVVNQQSAQYRLKHAF-200, SEQ ID NO: 20) (mutations relative to P19 (SEQ ID NO: 2) shown in bolded text; see e.g., FIG. 3A). As a preliminary screen mdx myoblasts were transduced in vitro and evaluated cell lysates by western blot (FIG. 3C). Qualitative western blot from transduced mdx cell lysates collected 96-hours post-delivery indicated that each redesign candidate expressed in a similar manner relative to the native Dys. Subsequently intramuscular administration of mdx^{4cv} mice into hindlimb muscles with rAAV6 vectors carrying either the native μDys or the M1-M5 redesign candidates. Hindlimb force production was assessed using the foot-pedal assay which incorporates 20 eccentric contractions over a period of ~10 min. During this protocol mdx^{4cv} typically display an ~50% reduction in force output by the 5th contraction while wildtype controls experience an ~10% reduction (FIG. 3D). As indicated by this assay, M5 performed least well (similar to untreated mdx^{4cv}), mdx^{4cv} having received MI or M3 vectors had an intermediate outcome, while mdx^{4cv} mice administered M2 and M4 vectors were indistinguishable from mice treated with native μDys or wildtype controls. Representative immunostaining for low (M5), intermediate (M1) and high (M2) functionally performing redesigns is indicated: dystrophin (green fluorescence), laminin (red fluorescence), and nuclei (blue fluorescence) (FIG. 3E). When tested for T cell reactivity, all but one of the redesigned peptides ablated the T cell response as determined by interferon gamma EliSpot assays (FIG. 4B). As with the exon-6 epitope, these assays used dystrophin-specific human T-cells derived from the same patient originally described in the aforementioned clinical trial.

### Combined Dual-Epitope Redesigns Promote Respiratory and Cardiac Functional Improvements in Aged Mdx^{4cv} Mice.

Diaphragm function was assessed in vivo non-invasively by sonography at 2, 4, and 8 months. At 2 months, differences between the native H3μDys and either D2M1 or D2M2 treated cohorts were not significant. During the experimental time course (2, 4 and 8 months), differences between wild-type control and mdx^{4cv} mice treated with H3μDys were not significant. Specifically, H3μDys provided 2, 4, 8 month displacement measurements (mm) of 0.856, 0.925, and 1.0; (n=5) while that of control yielded 0.852, 0.906, and 1.05; (n=5). However, at the 2-month time point the percent increase relative to untreated mdx^{4cv} mice represented 42.47%, 43.81%, 24.47%, and 30.93% for control, H3μDys, D2M1, and D2M2 respectively. While at 8 months, data indicated that D2M1 and D2M2 redesigns resulted in an intermediate performance relative to the mdx^{4cv} cohort treated with H2Dys where percentile increases in mdx^{4cv} mice were 56.73%, 27.58%, and 34.63% respectively for H3Dys, D2M1, and D2M2.

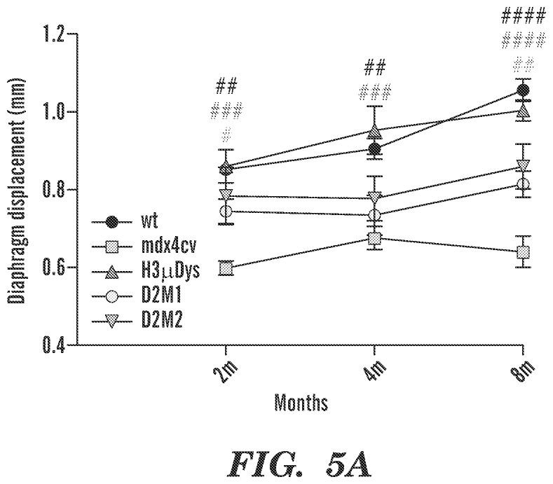

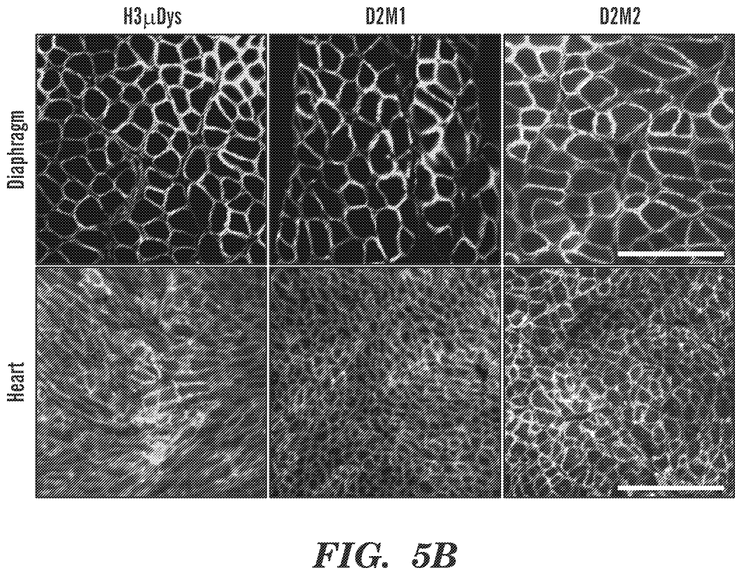

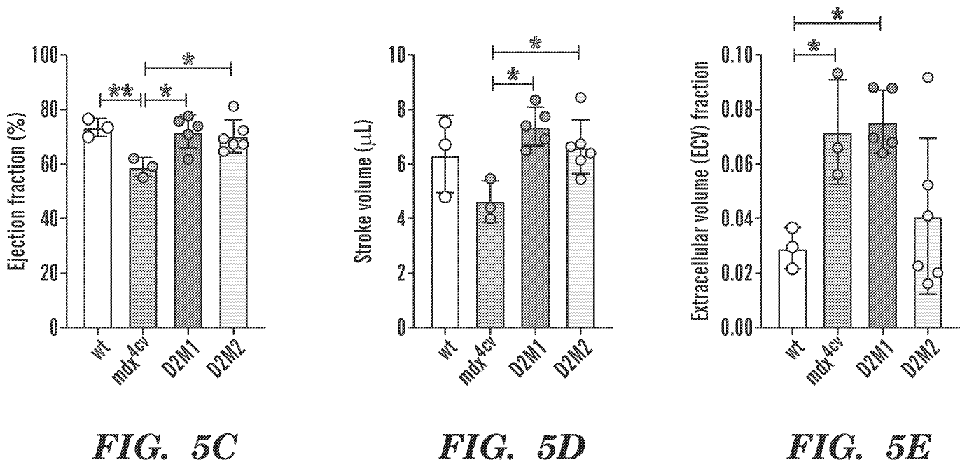

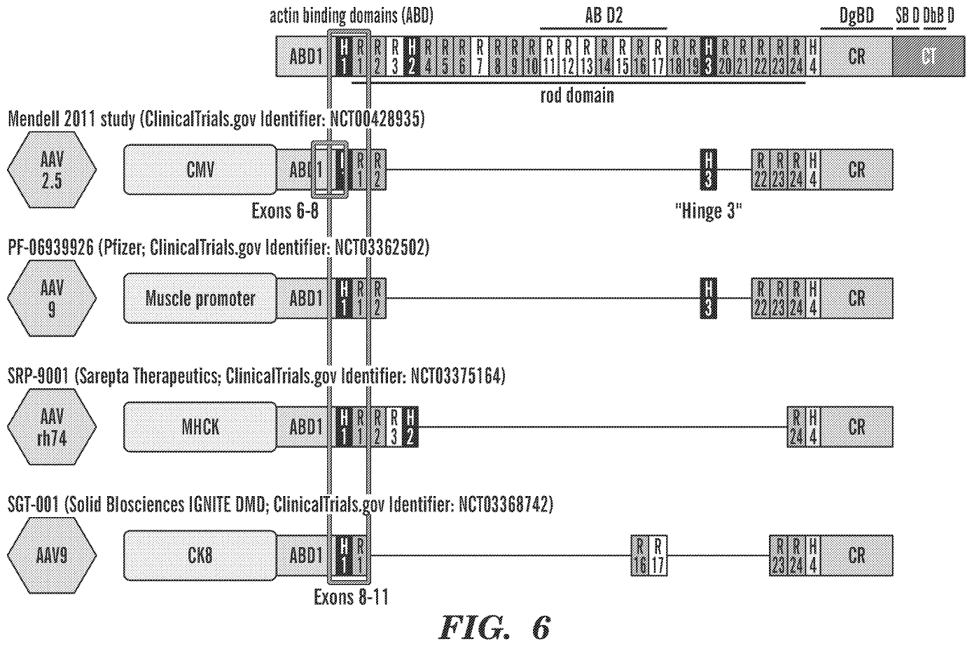

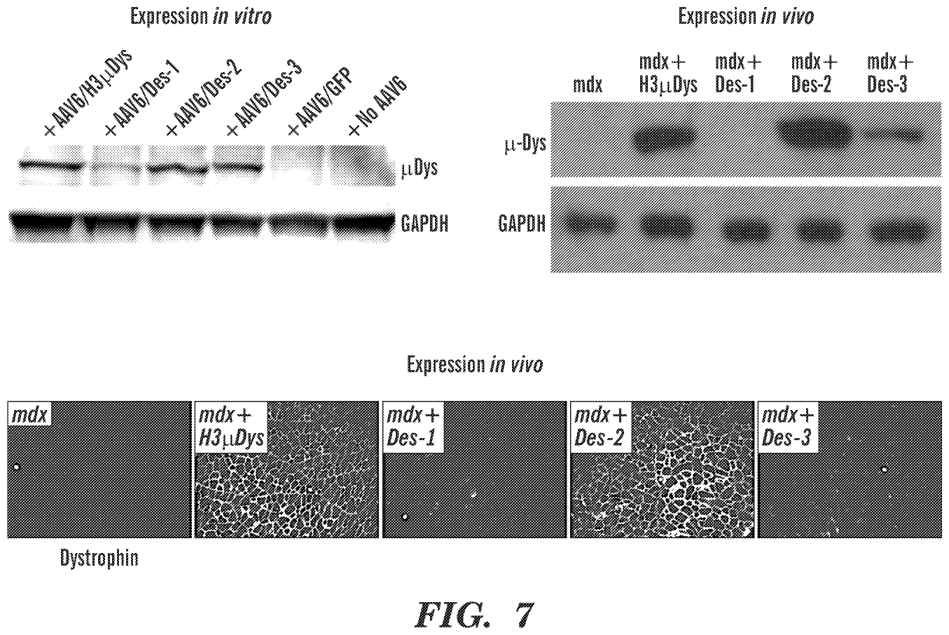

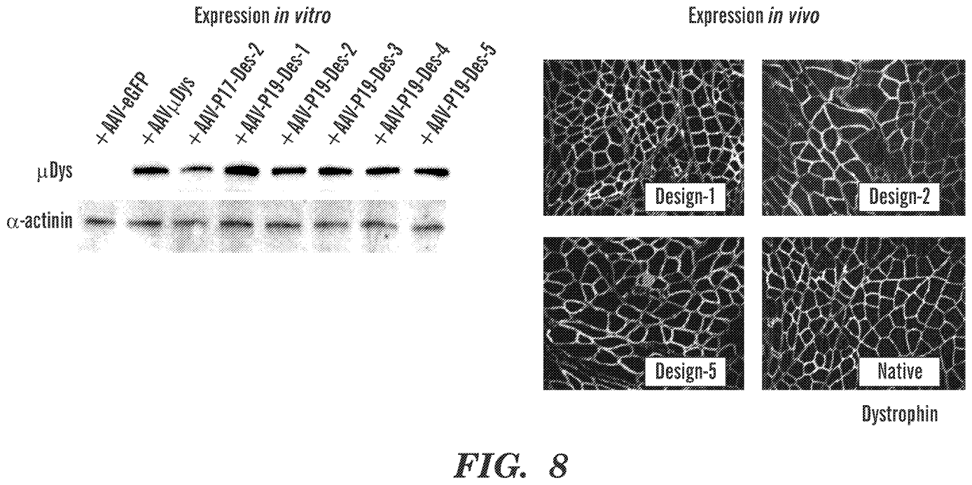

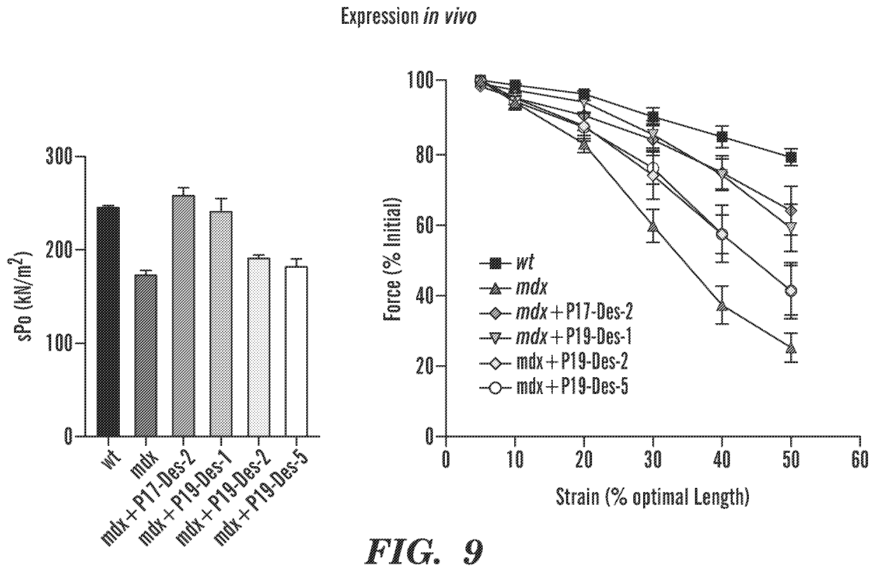

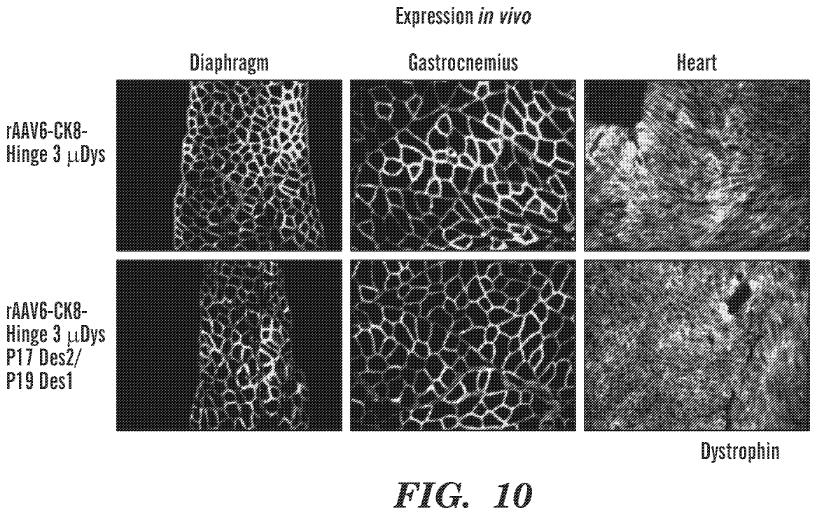

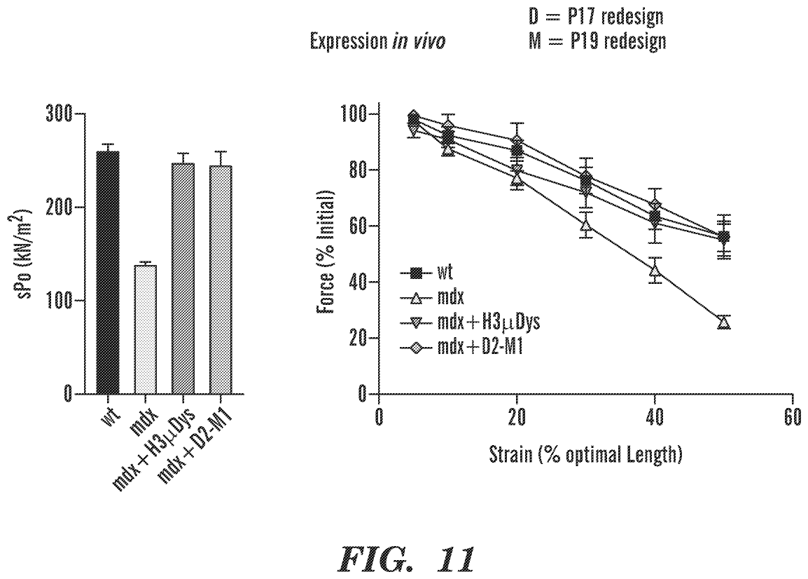

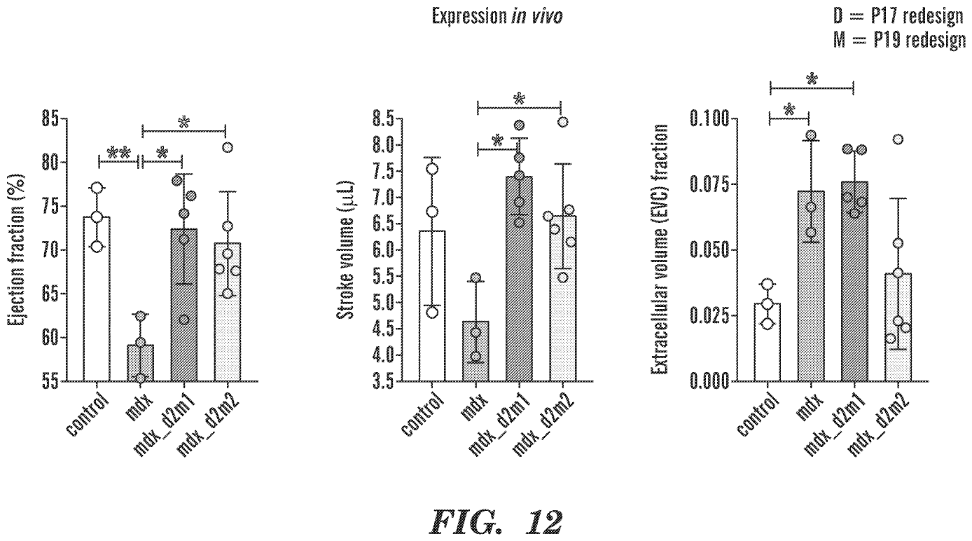

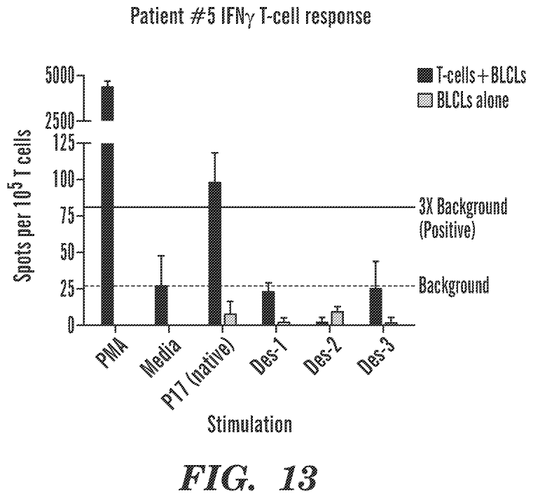

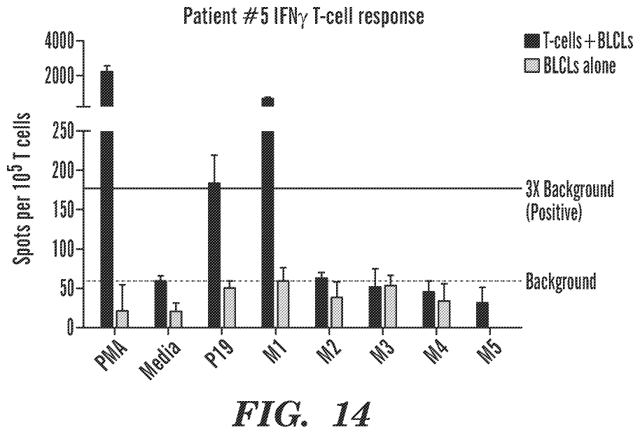

In an effort to address cardiac function four cohorts of mdx^{4cv} mice were evaluated at 17 months of age via cardiac magnetic resonance imaging (CMR). Here the two combined P17 (Des-2; D2) and P19 (M1 or M2) redesigns were evaluated for localized stability and functionality. By CMR, significant differences were noted between EF (%) levels of control and mdx^{4cv} mice (73.63±3.33% vs 59.07±3.54%, p<0.01). (FIG. 5C) Treatment with AAV-μDys improved ejection fraction (EF) in dystrophic myocardium (mdx^{4cv}; 59.07±3.54%, mdx^{4cv}+D2M1; 72.24±6.22% and mdx^{4cv}+D2M2; 70.66±5.91%, p<0.05). It should be noted that in the mdx mouse ejection fraction and other myocardium functional measures become abnormal after 11 months of age.^{45,46} One of the reasons for the decrease in EF (%) is the accumulation of collagenous/non-contractile tissue in the dystrophic myocardium. As such, the amount of the collagen fraction (ECV) was quantified using T1 weighted Gd contrast. The amount of ECV was significantly higher in untreated mdx^{4cv} mice compared to control mice (0.07±0.02 vs 0.03±0.007). Further, treatment with deimmunized AAV-μDys (mdx⁴″±D2M2) decreased ECV compared to untreated mdx^{4cv} myocardium (0.04±0.03 vs 0.07 ±0.02). These observations and measurements will be further explored and validated with comparisons to histological measurements in future studies for a more nuanced understanding of the cellular change. The data presented here demonstrate that AAV-mediated delivery of deimmunized μDys ameliorates cardiomyopathy of mdx^{4cv} mice and that CMR and ECV parameters may prove to be valuable in monitoring treatment effects in dystrophic myocardium. ECV mapping appears promising to compliment late gadolinium enhancement imaging.

### DISCUSSION

In many DMD patients, particularly those with large deletions or inversions, there has been a long-held concern of many in the gene therapy field that vector-delivered dystrophin proteins can be perceived as foreign and attacked by the immune system of DMD patients. Adding to the biological complexity, there are a variety of contributory factors affecting the potential for dystrophin specific immunity. For example, the already proinflammatory microenvironment within muscle(s), circulating surveillance immune cells, immune haplotype, and DMD mutation type of the patient, that together play a role in driving dystrophin specific immunity. In some patients there can be periodic protein expression (via revertant fibers-often lacking significant coding sequence) the consequence of which being not fully understood. On the other hand, many patients display undetectable dystrophin levels at least with limited biopsy preparations.

Indeed, a number of DMD patients are immune-reactive to dystrophin, even without dystrophin-expressing treatments, and multiple AAV gene therapy clinical trials have now seen evidence of immune responses against dystrophin. Chronologically, the first clinical trial (2005), in which patients received an intramuscular injection of AAV-CMV-μDys at Nationwide Children's Hospital (NCH), had four of six patients develop positive anti-dystrophin IFNγ ELISpots. Additionally, the immune response of some patients within the NCH trial was apparently boosted following AAV-μDys gene transfer, likely contributing to poor μDys expression in injected muscles.37 These data indicate that a number of patients are pre-immunized against dystrophin epitopes. These immune system issues are thought to have arisen in response to either rare somatic dystrophin mutations (escaping thymic maturation) or from revertant myofibers. It was speculated that the route of administration, concentrated dose within a single muscle, and CMV regulatory cassette were together largely responsible for these early events demonstrating dystrophin specific immunity. Not unlike vaccination protocols that are meant to evoke an immune response by intramuscular delivery with unbridled expression in antigen presenting cells.

Recently, five patients have been identified as having severe adverse events secondary to anti-dystrophin T cell immune responses in AAV clinical trials by PFIZER, SAREPTA, ROCHE (using Sarepta's vector) and GENETHON. A fifth trial by SOLID has had additional patients with positive IFNγ ELISpots against dystrophin, but no SAE's. Together, with AAV-mediated Dys administered intramuscularly or intravascularly, these studies demonstrate the potential of the immune system to respond to and occasionally reject dystrophin-expressing myotubes and cardiomyocytes, which could limit the efficacy of gene therapy treatments and cause irreparable harm to study participants. Based on patient mutation data of recent trials, these immune responses appear to be against protein domains encompassed by exons 8-11. Due to the severity of these events, patients with deletions that include these exons are being excluded from the gene therapy clinical trials, a devastating blow to families who have no other treatment options on the horizon. While one could attempt to induce tolerance to dystrophin in these patients, it may prove difficult in a pro-inflammatory dystrophic environment. Instead, the immunogenicity of the protein has been directly modulated with amino acid substitutions carefully chosen and screened to minimize their impact on function and eliminate dystrophin T cell responses to provide hope for the DMD community and those patients currently excluded from ongoing or future clinical trials.

As stated above, dystrophin specific immunity in DMD patients following gene transfer has been shown to occur in a number of patients as evidenced by multiple clinical trial efforts. Clinical details of which have been recently highlighted at various conferences with a correlation of antibody responses, ELISpot data, and patient mutation data. The retrospective study utilizing immune cells obtained from patient 5 (NCH clinical trial-NCT00428935) provided a unique opportunity and brought out a couple of details with regard to deimmunization for DMD. First, and importantly, the ability to deimmunize 2 epitopes was demonstrated (from 2 adjacent exons), previously identified within the former Nationwide Children's Hospital DMD clinical trial. Second, not all “deimmunized” redesigns can maintain near complete physiological functional activity as a result of unique biological challenges. One such challenge presents itself within the critical function of the ABD, registered with the adversely affected diaphragm in aged mdx^{4cv} mice. Indeed, missense mutations within the ABD have been known to occur but are not common. The results of attempts to re-design the P17 epitope are described herein. At one end of the scale is the leading candidate Des-2 re-design (FIG. 2A-2K, Des-2), which reduced immunogenicity and strongly preserved function, while at the other end of the scale resides a functional failure (P17, Des-1), which failed to prevent the dystrophic phenotype in mdx^{4cv} mice. In the middle, a category for intermediate performance emerged for the P17 epitope (Des-3). In the end, there was a 33% success rate to yield a deimmunized highly functional μDys for the P17 epitope.

Turning toward the P19 epitope, the ELISpot data (FIG. 4A-4B) for the P19 MI design yielded increased patient 5 T cell reactivity as indicated by IFNγ production while M2-M5 did not. With that said, the primary objective for advancement of P19 redesign-type(s) in the combined epitope (P17/P19) study was physiological performance. This is the same epitope previously identified by Walker and colleagues with reactivity of 2 sub-pools of synthetic peptides used in the ELISpot assay (albeit overlapping sequences) which mapped to within the P19 epitope (FDWNSVVCQQSATQRLEHAF, SEQ ID NO: 2). In both of those aforementioned sub-pools the CD8+ T cells matched target cells sharing HLA B*1801. As such the MHC I peptide binding groove restricts peptide lengths to 9 residues; and among the hundreds of HLAs in the Epitope Database, their cognate peptides exhibit 2 or 3 highly conserved anchor residues that account for most of the binding avidity to MHC I. The anchor residues are not always clearly defined, necessitating experimental coverage of a larger peptide region, which in turn can have unintended consequences on protein-protein interactions, for example.

With regard to P19 redesigns, if external mutations at positions 188 and 197 were excluded, both of which are mutated in M2-M5, without wishing to be bound by theory, a single residue mutation from an aspartate (M2 or M3) to a lysine (M1) can contribute to a dramatic reversal in T cell activation, as indicated by ELISpot outcomes. One difference at this position is the charge imposed at physiological pH with such a mutation. Normally, an uncharged glutamine occupies position 190; this is in contrast to the MI lysine mutation that imposes a basic side-chain with an α-amine group, while the mutation of M2 or M3 (aspartate) usually occurs as the negatively charged form (—COO) with its acidic side chain (CH2-COOH)). Positions 193-94 are mutated in all redesigns (KY in M1-M4 and QY in M5), indicating these positions were not negatively influencing the intended deimmunization. Although not specifically tested, it is hypothesized that positions 193-194 represent key anchor residues that provide sufficient destabilization to impart deimmunization of the P19 epitope. In an effort to test the therapeutic potential in the heart and diaphragm, a P17 redesign (Des-2) was combined with P19 redesign M1 or M2, and improved function was demonstrated by cMR, as evidenced by increased near normal ejection fraction for both D2M1 and D2M2. The extracellular volume (ECV) linearly related with fibrosis volume in the myocardium via Tl mapping, and with D2M2 indicated a decrease with values more similar to wildtype control. The combined epitope redesigns were deimmunized (for patient 5) and reached significant functional improvement in vivo. Such epitopes can be tested in further deimmunized candidates; their cardiorespiratory functional properties can be evaluated in multiple model systems.

The role of T cell immunity is a central driving force implicated in why this immune readiness occurs. However, the when and the how of such a response can often be muddied by complicating factors influencing the inherent immunity, such as the haplotype within an individual patient. Central to understanding an individual patient's immune response that can result in elimination of μDys positive myofibers is defining the primary epitope(s) responsible for evoking that response via MHC epitope binding and TCR recognition and signaling. In the absence of clearly defined known epitopes, prediction methods have greatly improved over the years in what has become a protein revolution, due to advances in computational technology, notwithstanding recent infusion of the ALPHAFOLD tool, improvements in immunological databases, and overall knowledge of structural/functional attributes of dystrophin. The dystrophin redesign approach described herein is targeted to the epitope core responsible for MHC-binding, as well as those 1 or 2 residues that anchor the epitope within the immune synapse. This in turn results in destabilization of the MHC: epitope binding efficacy and by extension prevents or greatly diminishes efficient MHC: epitope: TCR presentation, ultimately preventing dystrophin specific immune activation. The attack by the immune system can be pharmacologically navigable or manageable for a given patient, as developed immunosuppressive regiments have aided in deterring immunological complications in the clinic. However, lifelong immune suppression would not be ideal for a given patient. Furthermore, in those patients whose circumstances present with a SAE (or of a predictable risk category based on patient data) as a result of specific anti-dystrophin T cell responses, an alternative approach is provided.

These studies demonstrate that human dystrophin epitopes can be redesigned with the goal of deimmunization and that multiple versions can be developed for expression, functional and immunological studies. Adjacent exon epitopes can be combined and expressed long-term in mdx^{4cv} mice without significant loss of cardiac function. Taken together, the approach described herein can greatly facilitate the clinical application of safer and more efficacious gene therapies for DMD and other muscle disorders.

### Materials and Methods

### Animal Experiments

Male wildtype C57Bl/6J and mdx^{4cv} mice (THE JACKSON LABORATORY, Bar Harbor, ME) (n=4) were utilized for all studies. All animals were experimentally manipulated in accordance with the Institutional Animal Care and Use Committee (IACUC) of the University of Washington. To address “proof of principle” for dystrophin epitope deimmunization in early pilot experiments, two-week-old dystrophic mdx^{4cv} mice received either 1×10¹⁰ vector genomes (vg) of each redesigned rAAV6 vector in 30 μl (total volume) by intramuscular administration of the tibialis anterior or 2×10¹⁴ vg/kg intravenously, and were evaluated at 2, 8 or 18 months of age. These experiments were then followed by treatment of a larger cohort of three-week-old dystrophic mdx⁴⁰ mice that received 2×10¹² vg of the dual epitope redesign rAAV6 vectors or the native microdystrophin vector, via intravenous injection.

### Rosetta Deimmunization of Dystrophin

Atomic data for the human dystrophin (PDB: 1DXX), with a focus on exons 6-8, was interrogated with the ROSETTA protocol for structure-based deimmunization. Epitope anchor residues were selected for redesign on the basis of average predicted MHC binding affinity. In turn, all residues in any epitope frame that score above a certain threshold are selected for design. In consideration for downstream AAV-mediated in vivo functional activity screening, multiple design sequences were generated for each design position, these combined with one another in a stochastic approach providing a range in the mutation number and energetic impact.

### Construct Cloning and Vector Production

Recombinant AAV genomes containing an expression cassette for codon-optimized (GENSCRIPT, USA) microdystrophin (ΔH2-23/ΔCT+H3) and serotype 2 inverted terminal repeats were generated by standard cloning techniques. FseI and Bsu35I fragments containing deimmunized variants were synthesized (GENSCRIPT, USA) and subcloned into the actin-binding domain of the codon-optimized microdystrophin cDNA of the pUC57-coh-H3μDys. Microdystrophin variants were ultimately subcloned into pAAV-CK8e-H3μDys containing a muscle specific regulatory cassette based on conserved miniaturization of the murine muscle creatine kinase (MCK) gene (CK8e). Briefly, the CK8e cassette contains the mouse MCK enhancer-1240 through-1063 with a 63-bp deletion between the enhancer right E-box and MEF2 site; the MCK promoter-268 through+50 with a mutation of the transcription start site to conform to the consensus initiator sequence, and the inclusion of 50-bp from the MCK exon-1 5′UTR. Sal I+Hpa I fragments containing of the coding sequence for microdystrophins were sub-cloned into the plasmid native codon-optimized H3Dys backbone. The resulting plasmid constructs were co-transfected with the pDGM6 packaging plasmid into HEK293 cells to generate recombinant AAV vectors comprising serotype 6 capsids that were harvested, enriched, and quantitated as described previously. Enriched (up to ~2×10¹⁴ vg/ml) preparations of rAAV6 vector were stored in HANKS BUFFERED SALT SOLUTION (HBSS) at −80° C. as previously reported until thawed for use.

### Mdx Cultures and Myoblast Transduction

To evaluate relative expression levels of microdystrophin candidates utilizing mdx myoblasts (Hauschka lab-clone) plated at 500,000 cells per 60 mm plate on day 0. On day 1 at the 17 hour time point cells have nearly doubled, notably slower relative to MM14s (normal mouse myoblasts). The proliferative media was removed and cell plates were washed 2× with 3 ml of Saline-A, followed by the addition of 3 ml of F10 differentiation media containing 3% horse serum 10 mM Ca++. On day 3 the plates were washed 2× with Saline-A and fed with F10C supplemented with 5% HS. On day 4 the myotubes were washed, trypsinized, pelleted by centrifugation and resuspended in low ionic lysis buffer for immune assays.

### Immunoblots of Microdystrophins

Cell pellets were resuspended in 0.1 ml low-ionic solubilization buffer, samples were briefly incubated on wet ice (15 min) and centrifuged with supernatant collected, aliquoted and stored at −80° C. For tissue samples, muscle proteins were extracted in radioimmunoprecipitation analysis buffer (RIPA) supplemented with 2.5% protease inhibitor cocktail (SIGMA, St. Louis MO) and 5 mM EDTA. Samples were incubated on wet ice for 1 hour with periodic gentle mixing.

The PIERCE micro BCA protein assay kit (THERMOFISHER) was used to determine protein concentration. Protein lysates (20 μg) were denatured at 99 degrees Celsius for 10 min, quenched on ice and loaded to 10% pre-cast agarose gel (BIORAD) for gel electrophoresis with the CRITERION mini-gel apparatus (BIORAD). Protein transfer to 0.45 mm PVDF membranes was performed for 1 hour at constant 100 volts at 4C in TOWBIN'S buffer containing 20% methanol. Blots were blocked for 1 hour at room temperature in 5% non-fat dry milk in TBS-T before overnight incubation with antibodies raised against the FLAG-epitope (SIGMA, mouse monoclonal, 1:1000) and GAPDH (SIGMA, Rabbit polyclonal, 1:100,000). For secondary antibody incubation horseradish-peroxidase conjugated secondary antibody was used (JACKSON IMMUNORESEARCH, West Grove PA) staining (1:50,000) was performed for 1 h at room temperature before signal development using CLARITY Western ECL substrate (BIORAD) and visualization using a CHEMIDOC MP imaging system (BIORAD). Gel- and blot-band densitometry measurements were performed on unsaturated images using IMAGEJ software (National Institutes of Health).

### Skeletal Muscle Functional Analysis

Skeletal muscles were evaluated in situ for physiological performance prior to the mice being terminated for sample collection and further assay evaluation. Peak force was determined with optimized muscle length for contraction followed by an injury assay where the muscle is incrementally lengthened through its physiological responsive range. To determine functional improvements displayed in hindlimb (gastrocnemius) muscle, mice were analyzed at 8-months or 3-months post-injection having received treatment either intravenously or intramuscularly, respectively. Briefly, mice were anesthetized with isoflurane and assayed in situ for maximal force generation followed by protection from contraction-induced injury using methods and hardware described previously. To test the susceptibility of muscles to strain-mediated injury of varying intensity, individual muscle preparations (a minimum of 4 per cohort) were forcibly lengthened 5%, followed by 10% incremental increases beyond optimum length whilst maximally contracted a total of six times at 45 second intervals. In animals treated intravenously, muscles were further assayed for recovery force production following a 1-minute rest upon the final injury protocol lengthening contraction. Diaphragm physiological experiments were performed in vitro as previously described. Briefly, the diaphragm from wild-type (n=4), mdx^{4cv} (n=4), untreated mdx^{4cv}+native H3μDys (n=4) and mdx^{4cv}+di-H3μDys's (n=4) was dissected and placed in oxygenated KREBS (2 mM Ca²⁺, 24 mM NaHCO₃, 137 mM NaCl, 5 mM KCl, 1 mM MgSO4, 1 mM NaH2PO4, D-Glucose). Diaphragm strips were dissected from the left-medial tendon aspect of the muscle, sutured at either end, and the optimum length determined, followed by stimulation at 180 Hz, and recording peak tetanic contractile force measured over 350 ms. After contraction, the diaphragm strip was weighed, and specific force calculated as peak tetanic force production× length× density (1.04)× pennation (1.0 for the diaphragm)/muscle mass. Subsequently, prior to end-point assays diaphragm displacement was determined in anesthetized animals at 6, 18 and 32 weeks of age via sonography as previously described. Following diaphragm physiology, tissues were collected and processed for histological staining and dystrophin expression.

### Histological Analyses

Muscles were frozen in liquid nitrogen-cooled isopentane embedded in Tissue-Tek OCT medium (SAKURA FINETEK USA, Torrance, CA), and cut transversely in a cryostat at 10 μm. For brightfield microscopy, sections were fixed in methanol, hydrated and stained with hematoxylin and eosin followed by dehydration in ethanol, clearing in xylenes and permanently mounted. For immunofluorescence studies, sections were blocked in 4% bovine serum albumin and 0.05% Tween-20 in 1× DULBECCO'S phosphate buffered saline (PBS; INVITROGEN GIBCO, Grand Island, NY). Sections were washed three times in 1×PBS for 5 minutes each. A primary antibody incubation followed having an incubation of 60 minutes using ALEXA-488 conjugated anti-manex1011b dystrophin monoclonal antibody (University of Iowa, developmental studies hybridoma bank) with a 1:500 dilution, and rat anti-B2 laminin (1:800) (SIGMA, St. Louis, MO) in PBS containing 2% goat serum. The sections were washed three times in PBS and incubated for 45 minutes goat anti-rat-alexa-594 (1:800) (MOLECULAR PROBES INC., Eugene, OR). The sections were then mounted in anti-fade mounting media containing DAPI (SOUTHERN BIOTECH, Birmingham, AL). Fluorescent sections were imaged using a NIKON eclipse E1000 fluorescent microscope (NIKON, NY). For brightfield microscopy, sections were stained with hematoxylin and eosin and mounted with PERMOUNT (FISHER SCIENTIFIC, Fairlong, NJ). Images were captured using a QICAM digital camera and processed using QCAPTURE PRO (QIMAGING CORP.; BC Canada).

### Interferon Gamma (IFNγ) ELISpot Bioassays.

The IFN-γ ELISpot assays were set up in 96-well plates with capture antibody for our cytokine of interest (IFNγ). Incubation with conjugated secondary antibody to allow colorimetric detection with the alkaline phosphatase substrate (NBT/BCIP). Following coating wells with capture antibody, micro-dystrophin-specific semi-matched T-cells derived from subject 005 from an earlier clinical trial (NCT00428935) were cultured with the addition of synthetic peptides representing the native EXON-6 “P17” HLA-DQ7 epitope (163-WSDGLALNAL-172, SEQ ID NO: 1) in addition to 3 redesigned candidates carrying the sequences WSNGLGLDAL (SEQ ID NO: 7), WSDGTGLNAL (SEQ ID NO: 8), and LSDGTGLNAL (SEQ ID NO: 9), respectively. A second set of redesigned peptides was carried out for the exon-7 second epitope (“P19”) where 5 redesigns were evaluated via IFNγ ELISpot. Again, in a separate determinant assay the native peptide for the second epitope was included (180-FDWNSVVCQQSATQRLEHAF-200, SEQ ID NO: 2) along with five redesign candidates; FDWNSVVCQKSAKYRLEHAF (SEQ ID NO: 16), FDWNSVVQQDSAKYRLQHAF (SEQ ID NO: 17), FDWNSVVKQDSAKYRLKHAF (SEQ ID NO: 18), FDWDSVVQQQSAKYRLQHAF (SEQ ID NO: 19), and FDWNSVVNQQSAQYRLKHAF (SEQ ID NO: 20), respectively and referred to as M1-M5 hereafter. Following incubation at 37° C. for 36 hr γ-Interferon spot forming cells (SFC) were counted on an automatic ELISpot plate reader (FHCRC, Seattle WA). Responses were considered positive when SFC exceeded 50 per 106 APCs in duplicate wells.

Diaphragm ultrasonography. Animals were non-invasively imaged by ultrasonography to determine resultant diaphragm displacement (mm) as previously described. Briefly, the diaphragm movement amplitude was measured during each inspiration from the lowest point of the baseline, between contractions (5 successive contractions), to the peak level of the contraction. The movement amplitude was calculated as the difference between baseline and the displacement peak amplitude. Determination of time-dependent changes in dystrophic diaphragm function via sonography, in the presence or absence of vector were measured at 2, 4, and 8 months of age.

### Cardiac Magnetic Resonance Imaging.

Male wild type C57BL/6J (ctrl; n=3) and mdx^{4cv} (mdx⁴″; n=14) mice were utilized for the study. mdx^{4cv} (n=11) were treated with two altered variants of rAAV6-CK8-μDys (D2M1; n=5 and μDys D2M2; n=6) at 2 weeks of age with a dose of 4×10¹⁴ vg/kg. In vivo MRI was performed on a 14 tesla (T) NMR spectrometer (BRUKER BIOSPIN, Billerica, MA) at 17 months of age. Mice were anesthetized using 3% isoflurane and maintained at 1% isoflurane and a mixture of air and oxygen at a 3:1 ratio by using a vaporizer. The vertical bore of the magnet was maintained at 30° C. to maintain thermo-neutrality of the animal and respiratory rate was monitored and maintained at 50-70 breaths/min by adjusting the anesthetic concentration. The MR system was interfaced to a console running PARAVISION software 5.1 including the INTRAGATE software for sequence acquisition and reconstruction. Single slice coronal and sagittal long-axis scans (TR/TE=5/2.2 ms) were acquired to view apex and mitral valve planes. These long-axis scans were used to acquire 5 short-axis (TR/TE=5/2.2 ms) scans, which were then used to measure left ventricular function. To obtain the global parameters of the entire heart, the volume for each frame was calculated as the sum of the area of interest in each slice multiplied by the slice thickness. Ejection fraction (EF %) was calculated from the blood volume, determined in the end-systolic and end-diastolic cardiac cycle phases. Myocardial extracellular volume (ECV) was measured as the percent of tissue comprised of extracellular space⁵⁶. ECV was calculated using Tl-maps acquired pre and post-Gadolinium (Gd) contrast using the following parameters (TR/TE=5/2.2 ms, flip angle=2°, 5°, 10°, 20°, 40°) 60° and calibrated by blood hematocrit. Tl measurements were used to assess fibrosis formation as described, 57,58 In brief, ECV in % was determined using the following equation. ECV is linearly in others related to fibrosis volume.

$$ECV = ( 1 - hematocrit ) ( \frac{1 / {Post\_GdT1}_{m y o c a r d i u m} - 1 / {Pre\_GdT1}_{m y o c a r d i u m}}{1 / {Post\_GdT1}_{blood - pool} - 1 / {Pre\_GdT1}_{blood - pool}} ) \qquad Formula 1$$

T1 values in myocardium and blood pool were determined before and after Gd-DTPA injection. A hematocrit level of each mouse was measured on the blood sampled from the animal after MRI.

### Statistics:

All results are expressed as mean±s.d. unless otherwise stated. Differences identified between cohorts were determined using one-way ANOVA with a Tukey post-test that compares all data sets with a Student's t-test. All data analyses were performed using the PRISM software.

### REFERENCES

1. Emery, A. E. The muscular dystrophies. BMJ (Clinical research ed 317, 991-995 (1998).
2. Koenig, M., et al. Complete cloning of the Duchenne muscular dystrophy (DMD) cDNA and preliminary genomic organization of the DMD gene in normal and affected individuals. Cell 50, 509-517 (1987).
3. Ervasti, J. M. Structure and function of the dystrophin-glycoprotein complex. in Molecular Mechanisms of Muscular Dystrophies (ed. Winder, S. J.) 1-13 (Landes Biosciences, Georgetown, 2006).
4. Campbell, K. P. Three muscular dystrophies: loss of cytoskeleton-extracellular matrix linkage. Cell 80, 675-679 (1995).
5. Ervasti, J. M. & Campbell, K. P. A role for the dystrophin-glycoprotein complex as a transmembrane linker between laminin and actin. The Journal of cell biology 122, 809-823 (1993).
6. Ibraghimov-Beskrovnaya, O., et al. Primary structure of dystrophin-associated glycoproteins linking dystrophin to the extracellular matrix. Nature 355, 696-702 (1992).
7. Yoshida, M. & Ozawa, E. Glycoprotein complex anchoring dystrophin to sarcolemma. Journal of biochemistry 108, 748-752 (1990).
8. Abmayr, S. & Chamberlain, J. The structure and function of dystrophin. in The Molecular Mechanisms of Muscular Dystrophies (ed. Winder, S. J.) 14-34 (Landes Biosciences, Georgetown, 2006).
9. Harper, S. Q., et al. Modular flexibility of dystrophin: Implications for gene therapy of Duchenne muscular dystrophy. Nature Medicine 8, 253-261 (2002).
10. England, S. B., et al. Very mild muscular dystrophy associated with the deletion of 46% of dystrophin. Nature 343, 180-182 (1990).
11. Phelps, S. F., et al. Expression of full-length and truncated dystrophin mini-genes in transgenic mdx mice. Hum.Mol.Genet. 4, 1251-1258 (1995).
12. Crawford, G. E., et al. Assembly of the dystrophin-associated protein complex does not require the dystrophin COOH-terminal domain. Journal of Cell Biology 150, 1399-1410 (2000).
13. Wang, B., Li, J. & Xiao, X. Adeno-associated virus vector carrying human minidystrophin genes effectively ameliorates muscular dystrophy in mdx mouse model. Proceedings of the National Academy of Sciences of the United States of America 97, 13714-13719 (2000).
14. Sakamoto, M., et al. Micro-dystrophin cDNA ameliorates dystrophic phenotypes when introduced into mdx mice as a transgene. Biochem Biophys Res Commun 293, 1265-1272 (2002).
15. Chamberlain, J. S. Gene therapy of muscular dystrophy. Hum Mol Genet 11, 2355-2362 (2002).
16. Im, W. B., et al. Differential expression of dystrophin isoforms in strains of mdx mice with different mutations. Hum Mol Genet 5, 1149-1153 (1996).
17. Valentine, B. A., et al. Canine X-linked muscular dystrophy as an animal model of Duchenne muscular dystrophy: a review. Am.J.Med. Genet. 42, 352-356 (1992).
18. Le Guiner, C., et al. DYSTROPHIN deficient rats: generation and characterization of a new model for Duchenne muscular dystrophy. Hum Gene Ther 25, A28-A28 (2014).
19. Larcher, T., et al. Characterization of Dystrophin Deficient Rats: A New Model for Duchenne Muscular Dystrophy. Plos One 9 (2014).
20. Cox, G. A., et al. Overexpression of dystrophin in transgenic mdx mice eliminates dystrophic symptoms without toxicity [see comments]. Nature 364, 725-729 (1993).
21. Stedman, H. H., et al. The mdx mouse diaphragm reproduces the degenerative changes of Duchenne muscular dystrophy. Nature 352, 536-539 (1991).
22. Brooks, S. V. & Faulkner, J. A. Skeletal muscle weakness in old age: underlying mechanisms. Medical Science and Sports Exercise 26, 432-439 (1994).
23. DelloRusso, C., Crawford, R., Chamberlain, J. & Brooks, S. Tibialis anterior muscles of mdx mice are highly susceptible to contraction-induced injury. Journal of Muscle Research and Cell Motility 22, 467-475 (2001).
24. Lynch, G. S., Hinkle, R. T., Chamberlain, J. S., Brooks, S. V. & Faulkner, J. A. Force and power output of fast and slow skeletal muscles from mdx mice 6-28 months old. The Journal of physiology 535, 591-600 (2001).
25. Rabinowitz, J. E. & Samulski, J. Adeno-associated virus expression systems for gene transfer. Curr Opin Biotechnol 9, 470-475 (1998).
26. Weinmann, J., et al. Identification of a myotropic AAV by massively parallel in vivo evaluation of barcoded capsid variants. Nat Commun 11, 5432 (2020).
27. Tabebordbar, M., et al. Directed evolution of a family of AAV capsid variants enabling potent muscle-directed gene delivery across species. Cell 184, 4919-4938 e4922 (2021).
28. Chamberlain, J. S. A Boost for Muscle with Gene Therapy. N Engl J Med 386, 1184-1186 (2022).
29. Salva, M. Z., et al. Design of tissue-specific regulatory cassettes for high-level rAAV-mediated expression in skeletal and cardiac muscle. Mol Ther 15, 320-329 (2007).
30. Boisgerault, F., et al. Prolonged Gene Expression in Muscle Is Achieved Without Active Immune Tolerance Using MicrorRNA 142.3p-Regulated rAAV Gene Transfer. Hum Gene Ther 24, 393-405 (2013).
31. Wang, L. X., Dobrzynski, E., Schlachterman, A., Cao, O. & Herzog, R. W. Systemic protein delivery by muscle-gene transfer is limited by a local immune response. Blood 105, 4226-4234 (2005).
32. Forrest, S. M., et al. Further studies of gene deletions that cause Duchenne and Becker muscular dystrophies. Genomics 2, 109-114 (1988).
33. Den Dunnen, J. T., et al. Topography of the Duchenne muscular dystrophy (DMD) gene: FIGE and cDNA analysis of 194 cases reveals 115 deletions and 13 duplications. Am J Hum Genet 45, 835-847 (1989).
34. Pfizer. join.parentprojectmd.org/site/DocServer/A_Message_from_Pfizer_on_our_DMD_Clinical_Program-_Sept.pdf. (2021).
35. Wang, B., et al. Construction and analysis of compact muscle-specific promoters for AAV vectors. Gene Ther 15, 1489-1499 (2008).
36. Ramos, J. N., et al. Development of Novel Micro-dystrophins with Enhanced Functionality. Mol Ther 27, 623-635 (2019).
37. Mendell, J. R., et al. Dystrophin immunity in Duchenne's muscular dystrophy. N Engl J Med 363, 1429-1437 (2010).
38. He, Y., Rappuoli, R., De Groot, A. S. & Chen, R. T. Emerging vaccine informatics. J Biomed Biotechnol 2010, 218590 (2010).
39. King, C., et al. Removing T-cell epitopes with computational protein design. Proc Natl Acad Sci USA 111, 8577-8582 (2014).
40. Mazor, R., et al. Recombinant immunotoxin for cancer treatment with low immunogenicity by identification and silencing of human T-cell epitopes. Proc Natl Acad Sci USA 111, 8571-8576 (2014).
41. King, N. P., et al. Computational design of self-assembling protein nanomaterials with atomic level accuracy. Science 336, 1171-1174 (2012).
42. Fleishman, S. J. & Baker, D. Role of the biomolecular energy gap in protein design, structure, and evolution. Cell 149, 262-273 (2012).
43. Azoitei, M. L., et al. Computational design of high-affinity epitope scaffolds by backbone grafting of a linear epitope. J Mol Biol 415, 175-192 (2012).
44. Banks, G. B., Gregorevic, P., Allen, J. M., Finn, E., and Chamberlain, J. S. Functional capacity of dystrophins carrying deletions in the N-terminal actin-binding domain. Human Molecular Genetics 16, 2105-2113 (2007).
45. Quinlan, J. G., et al. Evolution of the mdx mouse cardiomyopathy: physiological and morphological findings. Neuromuscular disorders: NMD 14, 491-496 (2004).
46. Zhang, W., et al. Abnormal cardiac morphology, function and energy metabolism in the dystrophic mdx mouse: an MRI and MRS study. Journal of molecular and cellular cardiology 45, 754-760 (2008).
47. Flanigan, K. M., et al. Anti-dystrophin T cell responses in Duchenne muscular dystrophy: prevalence and a glucocorticoid treatment effect. Hum Gene Ther 24, 797-806 (2013).
48. Bonnemann, C. G., Belluscio, B. A., Braun, S., Morris, C. & Muntoni, F. A collaborative analysis by clinical trial sponsors and academic experts of anti-transgene SAEs in studies of gene therapy for DMD. Mol Ther 30, 4 (2022).
49. Henderson, D. M. L., A.; Ervasti, J. M. Disease-causing missense mutations in actin binding domain 1 of dystrophin induce thermodynamic instability and protein aggregation. Proceedings of the National Academy of Sciences of the United States of America 107, 9632-9637 (2010).
50. Rudolph, M. G., Stanfield, R. L. & Wilson, I. A. How TCRs bind MHCs, peptides, and coreceptors. Annu Rev Immunol 24, 419-466 (2006).
51. Altuvia, Y., Schueler, O. & Margalit, H. Ranking potential binding peptides to MHC molecules by a computational threading approach. J Mol Biol 249, 244-250 (1995).
52. Jumper, J., Evans, R., Pritzel, A. et al. Highly accurate protein structure prediction with AlphaFold. Nature 596, 583-589 (2021).
53. Gregorevic, P., et al. rAAV6-microdystrophin preserves muscle function and extends lifespan in severely dystrophic mice. Nature medicine 12, 787-789 (2006).
54. Banks, G. B., Combs, A. C., Odom, G. L., Bloch, R. J. & Chamberlain, J. S. Muscle structure influences utrophin expression in mdx mice. PLOS Genet 10, e1004431 (2014).
55. Whitehead, N. P., et al. Validation of ultrasonography for non-invasive assessment of diaphragm function in muscular dystrophy. The Journal of physiology 594, 7215-7227 (2016).
56. Kellman, P., Wilson, J. R., Xue, H., Ugander, M. & Arai, A. E. Extracellular volume fraction mapping in the myocardium, part 1: evaluation of an automated method. J Cardiovasc Magn Reson 14, 63 (2012).
57. Miller, C. A., et al. Comprehensive validation of cardiovascular magnetic resonance techniques for the assessment of myocardial extracellular volume. Circ Cardiovasc Imaging 6, 373-383 (2013).
58. Everett, R. J., et al. Assessment of myocardial fibrosis with Tl mapping MRI. Clin Radiol 71, 768-778 (2016).

### Sequences

SEQ ID NOs: 56-59 are exemplary full-length dystrophin sequences, which both have wild-type functions. SEQ ID NO: 56 and SEQ ID NO: 58 have 5 nucleic acid polymorphisms compared to each other, as shown in Table 2. SEQ ID NO: 57 and SEQ ID NO: 59 have 4 corresponding amino acid polymorphisms compared to each other, as shown in Table 2.

TABLE 2
Nucleic acid and amino acid polymorphisms between SEQ ID NOs: 56-59

| Nucleic | SEQ ID | SEQ ID | Corresponding | SEQ ID | SEQ ID NO: 59 | Dystrophin |
|---|---|---|---|---|---|---|
| acid | NO: 56 | NO: 58 | amino acid | NO: 57 | (NP_003997.2) | location |
| 2645 | G | A | 882 | Gly (G) | Asp (D) | Exon 21 |
| 4406 | T | A | 1469 | Leu (L) | Gln (Q) | SR 11 |
| 5530 | A | C | 1844 | Arg (R) | Arg (R) - no AA | Exon 39 |
| | | | | | change | |
| 7096 | C | A | 2366 | Gln (Q) | Lys (K) | Last codon of |
| | | | | | | exon 48 |
| 8810 | A | G | 2937 | Gln (Q) | Arg (R) | SR24 |

```
dystrophin coding sequence (CDS), 11058 nucleotides (nt)
SEQ ID NO: 56
ATGCTTTGGTGGGAAGAAGTAGAGGACTGTTATGAAAGAGAAGATGTTCAAAAGAAAAC
ATTCACAAAATGGGTAAATGCACAATTTTCTAAGTTTGGGAAGCAGCATATTGAGAACCTC
TTCAGTGACCTACAGGATGGGAGGCGCCTCCTAGACCTCCTCGAAGGCCTGACAGGGCAA
AAACTGCCAAAAGAAAAAGGATCCACAAGAGTTCATGCCCTGAACAATGTCAACAAGGC
ACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCA
AAAATGTAATGAAAAATATCATGGCTGGATTGCAACAAACCAACAGTGAAAAGATTCTCCT
GAGCTGGGTCCGACAATCAACTCGTAATTATCCACAGGTTAATGTAATCAACTTCACCACC
AGCTGGTCTGATGGCCTGGCTTTGAATGCTCTCATCCATAGTCATAGGCCAGACCTATTTGA
CTGGAATAGTGTGGTTTGCCAGCAGTCAGCCACACAACGACTGGAACATGCATTCAACAT
CGCCAGATATCAATTAGGCATAGAGAAACTACTCGATCCTGAAGATGTTGATACCACCTATC
CAGATAAGAAGTCCATCTTAATGTACATCACATCACTCTTCCAAGTTTTGCCTCAACAAGT
GAGCATTGAAGCCATCCAGGAAGTGGAAATGTTGCCAAGGCCACCTAAAGTGACTAAAG
AAGAACATTTTCAGTTACATCATCAAATGCACTATTCTCAACAGATCACGGTCAGTCTAGC
ACAGGGATATGAGAGAACTTCTTCCCCTAAGCCTCGATTCAAGAGCTATGCCTACACACAG
GCTGCTTATGTCACCACCTCTGACCCTACACGGAGCCCATTTCCTTCACAGCATTTGGAAG
CTCCTGAAGACAAGTCATTTGGCAGTTCATTGATGGAGAGTGAAGTAAACCTGGACCGTT
ATCAAACAGCTTTAGAAGAAGTATTATCGTGGCTTCTTTCTGCTGAGGACACATTGCAAGC
ACAAGGAGAGATTTCTAATGATGTGGAAGTGGTGAAAGACCAGTTTCATACTCATGAGGG
GTACATGATGGATTTGACAGCCCATCAGGGCCGGGTTGGTAATATTCTACAATTGGGAAGT
AAGCTGATTGGAACAGGAAAATTATCAGAAGATGAAGAAACTGAAGTACAAGAGCAGAT
GAATCTCCTAAATTCAAGATGGGAATGCCTCAGGGTAGCTAGCATGGAAAAACAAAGCAA
TTTACATAGAGTTTTAATGGATCTCCAGAATCAGAAACTGAAAGAGTTGAATGACTGGCTA
ACAAAAACAGAAGAAAGAACAAGGAAAATGGAGGAAGAGCCTCTTGGACCTGATCTTGA
AGACCTAAAACGCCAAGTACAACAACATAAGGTGCTTCAAGAAGATCTAGAACAAGAAC
AAGTCAGGGTCAATTCTCTCACTCACATGGTGGTGGTAGTTGATGAATCTAGTGGAGATCA
CGCAACTGCTGCTTTGGAAGAACAACTTAAGGTATTGGGAGATCGATGGGCAAACATCTG
TAGATGGACAGAAGACCGCTGGGTTCTTTTACAAGACATCCTTCTCAAATGGCAACGTCTT
ACTGAAGAACAGTGCCTTTTTAGTGCATGGCTTTCAGAAAAAGAAGATGCAGTGAACAAG
ATTCACACAACTGGCTTTAAAGATCAAAATGAAATGTTATCAAGTCTTCAAAAACTGGCCG
TTTTAAAAGCGGATCTAGAAAAGAAAAAGCAATCCATGGGCAAACTGTATTCACTCAAAC
AAGATCTTCTTTCAACACTGAAGAATAAGTCAGTGACCCAGAAGACGGAAGCATGGCTGG
ATAACTTTGCCCGGTGTTGGGATAATTTAGTCCAAAAACTTGAAAAGAGTACAGCACAGAT
TTCACAGGCTGTCACCACCACTCAGCCATCACTAACACAGACAACTGTAATGGAAACAGT
AACTACGGTGACCACAAGGGAACAGATCCTGGTAAAGCATGCTCAAGAGGAACTTCCAC
CACCACCTCCCCAAAAGAAGAGGCAGATTACTGTGGATTCTGAAATTAGGAAAAGGTTGG
ATGTTGATATAACTGAACTTCACAGCTGGATTACTCGCTCAGAAGCTGTGTTGCAGAGTCC
TGAATTTGCAATCTTTCGGAAGGAAGGCAACTTCTCAGACTTAAAAGAAAAAGTCAATGC
CATAGAGCGAGAAAAAGCTGAGAAGTTCAGAAAACTGCAAGATGCCAGCAGATCAGCTC
AGGCCCTGGTGGAACAGATGGTGAATGAGGGTGTTAATGCAGATAGCATCAAACAAGCCT
CAGAACAACTGAACAGCCGGTGGATCGAATTCTGCCAGTTGCTAAGTGAGAGACTTAACT
GGCTGGAGTATCAGAACAACATCATCGCTTTCTATAATCAGCTACAACAATTGGAGCAGAT
GACAACTACTGCTGAAAACTGGTTGAAAATCCAACCCACCACCCCATCAGAGCCAACAGC
AATTAAAAGTCAGTTAAAAATTTGTAAGGATGAAGTCAACCGGCTATCAGGTCTTCAACCT
CAAATTGAACGATTAAAAATTCAAAGCATAGCCCTGAAAGAGAAAGGACAAGGACCCATG
TTCCTGGATGCAGACTTTGTGGCCTTTACAAATCATTTTAAGCAAGTCTTTTCTGATGTGCA
GGCCAGAGAGAAAGAGCTACAGACAATTTTTGACACTTTGCCACCAATGCGCTATCAGGA
GACCATGAGTGCCATCAGGACATGGGTCCAGCAGTCAGAAACCAAACTCTCCATACCTCA
ACTTAGTGTCACCGACTATGAAATCATGGAGCAGAGACTCGGGGAATTGCAGGCTTTACA
AAGTTCTCTGCAAGAGCAACAAAGTGGCCTATACTATCTCAGCACCACTGTGAAAGAGAT
GTCGAAGAAAGCGCCCTCTGAAATTAGCCGGAAATATCAATCAGAATTTGAAGAAATTGA
GGGACGCTGGAAGAAGCTCTCCTCCCAGCTGGTTGAGCATTGTCAAAAGCTAGAGGAGC
AAATGAATAAACTCCGAAAAATTCAGAATCACATACAAACCCTGAAGAAATGGATGGCTG
AAGTTGATGTTTTTCTGAAGGAGGAATGGCCTGCCCTTGGGGATTCAGAAATTCTAAAAA
AGCAGCTGAAACAGTGCAGACTTTTAGTCAGTGATATTCAGACAATTCAGCCCAGTCTAA
ACAGTGTCAATGAAGGTGGGCAGAAGATAAAGAATGAAGCAGAGCCAGAGTTTGCTTCG
AGACTTGAGACAGAACTCAAAGAACTTAACACTCAGTGGGATCACATGTGCCAACAGGT
CTATGCCAGAAAGGAGGCCTTGAAGGGAGGTTTGGAGAAAACTGTAAGCCTCCAGAAAG
ATCTATCAGAGATGCACGAATGGATGACACAAGCTGAAGAAGAGTATCTTGAGAGAGATT
TTGAATATAAAACTCCAGATGAATTACAGAAAGCAGTTGAAGAGATGAAGAGAGCTAAAG
AAGAGGCCCAACAAAAAGAAGCGAAAGTGAAACTCCTTACTGAGTCTGTAAATAGTGTC
ATAGCTCAAGCTCCACCTGTAGCACAAGAGGCCTTAAAAAAGGAACTTGAAACTCTAACC
ACCAACTACCAGTGGCTCTGCACTAGGCTGAATGGGAAATGCAAGACTTTGGAAGAAGTT
TGGGCATGTTGGCATGAGTTATTGTCATACTTGGAGAAAGCAAACAAGTGGCTAAATGAA
GTAGAATTTAAACTTAAAACCACTGAAAACATTCCTGGCGGAGCTGAGGAAATCTCTGAG
GTGCTAGATTCACTTGAAAATTTGATGCGACATTCAGAGGATAACCCAAATCAGATTCGCA
TATTGGCACAGACCCTAACAGATGGCGGAGTCATGGATGAGCTAATCAATGAGGAACTTG
AGACATTTAATTCTCGTTGGAGGGAACTACATGAAGAGGCTGTAAGGAGGCAAAAGTTGC
TTGAACAGAGCATCCAGTCTGCCCAGGAGACTGAAAAATCCTTACACTTAATCCAGGAGT
CCCTCACATTCATTGACAAGCAGTTGGCAGCTTATATTGCAGACAAGGTGGACGCAGCTCA
AATGCCTCAGGAAGCCCAGAAAATCCAATCTGATTTGACAAGTCATGAGATCAGTTTAGA
AGAAATGAAGAAACATAATCAGGGGAAGGAGGCTGCCCAAAGAGTCCTGTCTCAGATTG
ATGTTGCACAGAAAAAATTACAAGATGTCTCCATGAAGTTTCGATTATTCCAGAAACCAGC
CAATTTTGAGCTGCGTCTACAAGAAAGTAAGATGATTTTAGATGAAGTGAAGATGCACTTG
CCTGCATTGGAAACAAAGAGTGTGGAACAGGAAGTAGTACAGTCACAGCTAAATCATTGT
GTGAACTTGTATAAAAGTCTGAGTGAAGTGAAGTCTGAAGTGGAAATGGTGATAAAGACT
GGACGTCAGATTGTACAGAAAAAGCAGACGGAAAATCCCAAAGAACTTGATGAAAGAGT
AACAGCTTTGAAATTGCATTATAATGAGCTGGGAGCAAAGGTAACAGAAAGAAAGCAACA
GTTGGAGAAATGCTTGAAATTGTCCCGTAAGATGCGAAAGGAAATGAATGTCTTGACAGA
ATGGCTGGCAGCTACAGATATGGAATTGACAAAGAGATCAGCAGTTGAAGGAATGCCTAG
TAATTTGGATTCTGAAGTTGCCTGGGGAAAGGCTACTCAAAAAGAGATTGAGAAACAGAA
GGTGCACCTGAAGAGTATCACAGAGGTAGGAGAGGCCTTGAAAACAGTTTTGGGCAAGA
AGGAGACGTTGGTGGAAGATAAACTCAGTCTTCTGAATAGTAACTGGATAGCTGTCACCTC
CCGAGCAGAAGAGTGGTTAAATCTTTTGTTGGAATACCAGAAACACATGGAAACTTTTGA
CCAGAATGTGGACCACATCACAAAGTGGATCATTCAGGCTGACACACTTTTGGATGAATC
AGAGAAAAAGAAACCCCAGCAAAAAGAAGACGTGCTTAAGCGTTTAAAGGCAGAACTG
AATGACATACGCCCAAAGGTGGACTCTACACGTGACCAAGCAGCAAACTTGATGGCAAAC
CGCGGTGACCACTGCAGGAAATTAGTAGAGCCCCAAATCTCAGAGCTCAACCATCGATTT
GCAGCCATTTCACACAGAATTAAGACTGGAAAGGCCTCCATTCCTTTGAAGGAATTGGAG
CAGTTTAACTCAGATATACAAAAATTGCTTGAACCACTGGAGGCTGAAATTCAGCAGGGG
GTGAATCTGAAAGAGGAAGACTTCAATAAAGATATGAATGAAGACAATGAGGGTACTGTA
AAAGAATTGTTGCAAAGAGGAGACAACTTACAACAAAGAATCACAGATGAGAGAAAGAG
AGAGGAAATAAAGATAAAACAGCAGCTGTTACAGACAAAACATAATGCTCTCAAGGATTT
GAGGTCTCAAAGAAGAAAAAAGGCTCTAGAAATTTCTCATCAGTGGTATCAGTACAAGAG
GCAGGCTGATGATCTCCTGAAATGCTTGGATGACATTGAAAAAAAATTAGCCAGCCTACCT
GAGCCCAGAGATGAAAGGAAAATAAAGGAAATTGATCGGGAATTGCAGAAGAAGAAAGA
GGAGCTGAATGCAGTGCGTAGGCAAGCTGAGGGCTTGTCTGAGGATGGGGCCGCAATGG
CAGTGGAGCCAACTCAGATCCAGCTCAGCAAGCGCTGGCGGGAAATTGAGAGCAAATTT
GCTCAGTTTCGAAGACTCAACTTTGCACAAATTCACACTGTCCGTGAAGAAACGATGATG
GTGATGACTGAAGACATGCCTTTGGAAATTTCTTATGTGCCTTCTACTTATTTGACTGAAAT
CACTCATGTCTCACAAGCCCTATTAGAAGTGGAACAACTTCTCAATGCTCCTGACCTCTGT
GCTAAGGACTTTGAAGATCTCTTTAAGCAAGAGGAGTCTCTGAAGAATATAAAAGATAGTC
TACAACAAAGCTCAGGTCGGATTGACATTATTCATAGCAAGAAGACAGCAGCATTGCAAA
GTGCAACGCCTGTGGAAAGGGTGAAGCTACAGGAAGCTCTCTCCCAGCTTGATTTCCAAT
GGGAAAAAGTTAACAAAATGTACAAGGACCGACAAGGGCGATTTGACAGATCTGTTGAG
AAATGGCGGCGTTTTCATTATGATATAAAGATATTTAATCAGTGGCTAACAGAAGCTGAACA
GTTTCTCAGAAAGACACAAATTCCTGAGAATTGGGAACATGCTAAATACAAATGGTATCTT
AAGGAACTCCAGGATGGCATTGGGCAGCGGCAAACTGTTGTCAGAACATTGAATGCAACT
GGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAG
GCTAGAAGAACAAAAGAATATCTTGTCAGAATTTCAAAGAGATTTAAATGAATTTGTTTTA
TGGTTGGAGGAAGCAGATAACATTGCTAGTATCCCACTTGAACCTGGAAAAGAGCAGCAA
CTAAAAGAAAAGCTTGAGCAAGTCAAGTTACTGGTGGAAGAGTTGCCCCTGCGCCAGGG
AATTCTCAAACAATTAAATGAAACTGGAGGACCCGTGCTTGTAAGTGCTCCCATAAGCCCA
GAAGAGCAAGATAAACTTGAAAATAAGCTCAAGCAGACAAATCTCCAGTGGATAAAGGTT
TCCAGAGCTTTACCTGAGAAACAAGGAGAAATTGAAGCTCAAATAAAAGACCTTGGGCA
GCTTGAAAAAAAGCTTGAAGACCTTGAAGAGCAGTTAAATCATCTGCTGCTGTGGTTATCT
CCTATTAGGAATCAGTTGGAAATTTATAACCAACCAAACCAAGAAGGACCATTIGACGTTC
AGGAAACTGAAATAGCAGTTCAAGCTAAACAACCGGATGTGGAAGAGATTTTGTCTAAAG
GGCAGCATTTGTACAAGGAAAAACCAGCCACTCAGCCAGTGAAGAGGAAGTTAGAAGAT
CTGAGCTCTGAGTGGAAGGCGGTAAACCGTTTACTTCAAGAGCTGAGGGCAAAGCAGCC
TGACCTAGCTCCTGGACTGACCACTATTGGAGCCTCTCCTACTCAGACTGTTACTCTGGTG
ACACAACCTGTGGTTACTAAGGAAACTGCCATCTCCAAACTAGAAATGCCATCTTCCTTGA
TGTTGGAGGTACCTGCTCTGGCAGATTTCAACCGGGCTTGGACAGAACTTACCGACTGGC
TTTCTCTGCTTGATCAAGTTATAAAATCACAGAGGGTGATGGTGGGTGACCTTGAGGATAT
CAACGAGATGATCATCAAGCAGAAGGCAACAATGCAGGATTTGGAACAGAGGCGTCCCC
AGTTGGAAGAACTCATTACCGCTGCCCAAAATTTGAAAAACAAGACCAGCAATCAAGAG
GCTAGAACAATCATTACGGATCGAATTGAAAGAATTCAGAATCAGTGGGATGAAGTACAA
GAACACCTTCAGAACCGGAGGCAACAGTTGAATGAAATGTTAAAGGATTCAACACAATGG
CTGGAAGCTAAGGAAGAAGCTGAGCAGGTCTTAGGACAGGCCAGAGCCAAGCTTGAGTC
ATGGAAGGAGGGTCCCTATACAGTAGATGCAATCCAAAAGAAAATCACAGAAACCAAGCA
GTTGGCCAAAGACCTCCGCCAGTGGCAGACAAATGTAGATGTGGCAAATGACTTGGCCCT
GAAACTTCTCCGGGATTATTCTGCAGATGATACCAGAAAAGTCCACATGATAACAGAGAAT
ATCAATGCCTCTTGGAGAAGCATTCATAAAAGGGTGAGTGAGCGAGAGGCTGCTTTGGAA
GAAACTCATAGATTACTGCAACAGTTCCCCCTGGACCTGGAAAAGTTTCTTGCCTGGCTTA
CAGAAGCTGAAACAACTGCCAATGTCCTACAGGATGCTACCCGTAAGGAAAGGCTCCTAG
AAGACTCCAAGGGAGTAAAAGAGCTGATGAAACAATGGCAAGACCTCCAAGGTGAAATT
GAAGCTCACACAGATGTTTATCACAACCTGGATGAAAACAGCCAAAAAATCCTGAGATCC
CTGGAAGGTTCCGATGATGCAGTCCTGTTACAAAGACGTTTGGATAACATGAACTTCAAGT
GGAGTGAACTTCGGAAAAAGTCTCTCAACATTAGGTCCCATTTGGAAGCCAGTTCTGACC
AGTGGAAGCGTCTGCACCTTTCTCTGCAGGAACTTCTGGTGTGGCTACAGCTGAAAGATG
ATGAATTAAGCCGGCAGGCACCTATTGGAGGCGACTTTCCAGCAGTTCAGAAGCAGAACG
ATGTACATAGGGCCTTCAAGAGGGAATTGAAAACTAAAGAACCTGTAATCATGAGTACTCT
TGAGACTGTACGAATATTTCTGACAGAGCAGCCTTTGGAAGGACTAGAGAAACTCTACCA
GGAGCCCAGAGAGCTGCCTCCTGAGGAGAGAGCCCAGAATGTCACTCGGCTTCTACGAA
AGCAGGCTGAGGAGGTCAATACTGAGTGGGAAAAATTGAACCTGCACTCCGCTGACTGG
CAGAGAAAAATAGATGAGACCCTTGAAAGACTCCAGGAACTTCAAGAGGCCACGGATGA
GCTGGACCTCAAGCTGCGCCAAGCTGAGGTGATCAAGGGATCCTGGCAGCCCGTGGGCG
ATCTCCTCATTGACTCTCTCCAAGATCACCTCGAGAAAGTCAAGGCACTTCGAGGAGAAA
TTGCGCCTCTGAAAGAGAACGTGAGCCACGTCAATGACCTTGCTCGCCAGCTTACCACTT
TGGGCATTCAGCTCTCACCGTATAACCTCAGCACTCTGGAAGACCTGAACACCAGATGGA
AGCTTCTGCAGGTGGCCGTCGAGGACCGAGTCAGGCAGCTGCATGAAGCCCACAGGGAC
TTTGGTCCAGCATCTCAGCACTTTCTTTCCACGTCTGTCCAGGGTCCCTGGGAGAGAGCCA
TCTCGCCAAACAAAGTGCCCTACTATATCAACCACGAGACTCAAACAACTTGCTGGGACC
ATCCCAAAATGACAGAGCTCTACCAGTCTTTAGCTGACCTGAATAATGTCAGATTCTCAGC
TTATAGGACTGCCATGAAACTCCGAAGACTGCAGAAGGCCCTTTGCTTGGATCTCTTGAGC
CTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAA
CAATTTGGTCAACGTCCCTCTCTGCGTGGATATGTGTCTGAACTGGCTGCTGAATGTTTATG
ATACGGGACGAACAGGGAGGATCCGTGTCCTGTCTTTTAAAACTGGCATCATTTCCCTGTG
TAAAGCACATTTGGAAGACAAGTACAGATACCTTTTCAAGCAAGTGGCAAGTTCAACAGG
ATTTTGTGACCAGCGCAGGCTGGGCCTCCTTCTGCATGATTCTATCCAAATTCCAAGACAG
TTGGGTGAAGTTGCATCCTTTGGGGGCAGTAACATTGAGCCAAGTGTCCGGAGCTGCTTC
CAATTTGCTAATAATAAGCCAGAGATCGAAGCGGCCCTCTTCCTAGACTGGATGAGACTGG
AACCCCAGTCCATGGTGTGGCTGCCCGTCCTGCACAGAGTGGCTGCTGCAGAAACTGCCA
AGCATCAGGCCAAATGTAACATCTGCAAAGAGTGTCCAATCATTGGATTCAGGTACAGGA
GTCTAAAGCACTTTAATTATGACATCTGCCAAAGCTGCTTTTTTTCTGGTCGAGTTGCAAA
AGGCCATAAAATGCACTATCCCATGGTGGAATATTGCACTCCGACTACATCAGGAGAAGAT
GTTCGAGACTTTGCCAAGGTACTAAAAAACAAATTTCGAACCAAAAGGTATTTTGCGAAG
CATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTC
ACACGATGATACTCATTCACGCATTGAACATTATGCTAGCAGGCTAGCAGAAATGGAAAAC
AGCAATGGATCTTATCTAAATGATAGCATCTCTCCTAATGAGAGCATAGATGATGAACATTT
GTTAATCCAGCATTACTGCCAAAGTTTGAACCAGGACTCCCCCCTGAGCCAGCCTCGTAGT
CCTGCCCAGATCTTGATTTCCTTAGAGAGTGAGGAAAGAGGGGAGCTAGAGAGAATCCTA
GCAGATCTTGAGGAAGAAAACAGGAATCTGCAAGCAGAATATGACCGTCTAAAGCAGCA
GCACGAACATAAAGGCCTGTCCCCACTGCCGTCCCCTCCTGAAATGATGCCCACCTCTCCC
CAGAGTCCCCGGGATGCTGAGCTCATTGCTGAGGCCAAGCTACTGCGTCAACACAAAGGC
CGCCTGGAAGCCAGGATGCAAATCCTGGAAGACCACAATAAACAGCTGGAGTCACAGTTA
CACAGGCTAAGGCAGCTGCTGGAGCAACCCCAGGCAGAGGCCAAAGTGAATGGCACAAC
GGTGTCCTCTCCTTCTACCTCTCTACAGAGGTCCGACAGCAGTCAGCCTATGCTGCTCCGA
GTGGTTGGCAGTCAAACTTCGGACTCCATGGGTGAGGAAGATCTTCTCAGTCCTCCCCAG
GACACAAGCACAGGGTTAGAGGAGGTGATGGAGCAACTCAACAACTCCTTCCCTAGTTCA
AGAGGAAGAAATACCCCTGGAAAGCCAATGAGAGAGGACACAATGTAG,
dystrophin polypeptide, 3685 amino acids (aa); Sequence Motif A (163-
WSDGLALNAL-172, SEQ ID NO: 1) and Sequence Motif B (181-
FDWNSVVCQQSATQRLEHAF-200, SEQ ID NO: 2) are bolded and double-underlined:
SEQ ID NO: 57
MLWWEEVEDCYEREDVQKKTFTKWVNAQFSKFGKQHIENLFSDLQDGRRLLDLLEGLTGQ
KLPKEKGSTRVHALNNVNKALRVLQNNNVDLVNIGSTDIVDGNHKLTLGLIWNIILHWQVK
NVMKNIMAGLQQTNSEKILLSWVRQSTRNYPQVNVINFTTSWSDGLALNALIHSHRPDLFD
WNSVVCQQSATQRLEHAFNIARYQLGIEKLLDPEDVDTTYPDKKSILMYITSLFQVLPQQVS
IEAIQEVEMLPRPPKVTKEEHFQLHHQMHYSQQITVSLAQGYERTSSPKPRFKSYAYTQAAY
VTTSDPTRSPFPSQHLEAPEDKSFGSSLMESEVNLDRYQTALEEVLSWLLSAEDTLQAQGEIS
NDVEVVKDQFHTHEGYMMDLTAHQGRVGNILQLGSKLIGTGKLSEDEETEVQEQMNLLNSR
WECLRVASMEKQSNLHRVLMDLQNQKLKELNDWLTKTEERTRKMEEEPLGPDLEDLKRQV
QQHKVLQEDLEQEQVRVNSLTHMVVVVDESSGDHATAALEEQLKVLGDRWANICRWTEDR
WVLLQDILLKWQRLTEEQCLFSAWLSEKEDAVNKIHTTGFKDQNEMLSSLQKLAVLKADLE
KKKQSMGKLYSLKQDLLSTLKNKSVTQKTEAWLDNFARCWDNLVQKLEKSTAQISQAVTT
TQPSLTQTTVMETVTTVTTREQILVKHAQEELPPPPPQKKRQITVDSEIRKRLDVDITELHSWI
TRSEAVLQSPEFAIFRKEGNFSDLKEKVNAIEREKAEKFRKLQDASRSAQALVEQMVNEGVN
ADSIKQASEQLNSRWIEFCQLLSERLNWLEYQNNIIAFYNQLQQLEQMTTTAENWLKIQPTTP
SEPTAIKSQLKICKDEVNRLSGLQPQIERLKIQSIALKEKGQGPMFLDADFVAFTNHFKQVFSD
VQAREKELQTIFDTLPPMRYQETMSAIRTWVQQSETKLSIPQLSVTDYEIMEQRLGELQALQS
SLQEQQSGLYYLSTTVKEMSKKAPSEISRKYQSEFEEIEGRWKKLSSQLVEHCQKLEEQMNK
LRKIQNHIQTLKKWMAEVDVFLKEEWPALGDSEILKKQLKQCRLLVSDIQTIQPSLNSVNEG
GQKIKNEAEPEFASRLETELKELNTQWDHMCQQVYARKEALKGGLEKTVSLQKDLSEMHE
WMTQAEEEYLERDFEYKTPDELQKAVEEMKRAKEEAQQKEAKVKLLTESVNSVIAQAPPVA
QEALKKELETLTTNYQWLCTRLNGKCKTLEEVWACWHELLSYLEKANKWLNEVEFKLKTT
ENIPGGAEEISEVLDSLENLMRHSEDNPNQIRILAQTLTDGGVMDELINEELETFNSRWRELHE
EAVRRQKLLEQSIQSAQETEKSLHLIQESLTFIDKQLAAYIADKVDAAQMPQEAQKIQSDLTS
HEISLEEMKKHNQGKEAAQRVLSQIDVAQKKLQDVSMKFRLFQKPANFELRLQESKMILDE
VKMHLPALETKSVEQEVVQSQLNHCVNLYKSLSEVKSEVEMVIKTGRQIVQKKQTENPKEL
DERVTALKLHYNELGAKVTERKQQLEKCLKLSRKMRKEMNVLTEWLAATDMELTKRSAVE
GMPSNLDSEVAWGKATQKEIEKQKVHLKSITEVGEALKTVLGKKETLVEDKLSLLNSNWIA
VTSRAEEWLNLLLEYQKHMETFDQNVDHITKWIIQADTLLDESEKKKPQQKEDVLKRLKAE
LNDIRPKVDSTRDQAANLMANRGDHCRKLVEPQISELNHRFAAISHRIKTGKASIPLKELEQF
NSDIQKLLEPLEAEIQQGVNLKEEDFNKDMNEDNEGTVKELLQRGDNLQQRITDERKREEIKI
KQQLLQTKHNALKDLRSQRRKKALEISHQWYQYKRQADDLLKCLDDIEKKLASLPEPRDER
KIKEIDRELQKKKEELNAVRRQAEGLSEDGAAMAVEPTQIQLSKRWREIESKFAQFRRLNFA
QIHTVREETMMVMTEDMPLEISYVPSTYLTEITHVSQALLEVEQLLNAPDLCAKDFEDLFKQE
ESLKNIKDSLQQSSGRIDIIHSKKTAALQSATPVERVKLQEALSQLDFQWEKVNKMYKDRQG
RFDRSVEKWRRFHYDIKIFNQWLTEAEQFLRKTQIPENWEHAKYKWYLKELQDGIGQRQTV
VRTLNATGEEIIQQSSKTDASILQEKLGSLNLRWQEVCKQLSDRKKRLEEQKNILSEFQRDLN
EFVLWLEEADNIASIPLEPGKEQQLKEKLEQVKLLVEELPLRQGILKQLNETGGPVLVSAPISP
EEQDKLENKLKQTNLQWIKVSRALPEKQGEIEAQIKDLGQLEKKLEDLEEQLNHLLLWLSPIR
NQLEIYNQPNQEGPFDVQETEIAVQAKQPDVEEILSKGQHLYKEKPATQPVKRKLEDLSSEW
KAVNRLLQELRAKQPDLAPGLTTIGASPTQTVTLVTQPVVTKETAISKLEMPSSLMLEVPALA
DFNRAWTELTDWLSLLDQVIKSQRVMVGDLEDINEMIIKQKATMQDLEQRRPQLEELITAAQ
NLKNKTSNQEARTIITDRIERIQNQWDEVQEHLQNRRQQLNEMLKDSTQWLEAKEEAEQVL
GQARAKLESWKEGPYTVDAIQKKITETKQLAKDLRQWQTNVDVANDLALKLLRDYSADDT
RKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRL
DNMNFKWSELRKKSLNIRSHLEASSDQWKRLHLSLQELLVWLQLKDDELSRQAPIGGDFPA
VQKQNDVHRAFKRELKTKEPVIMSTLETVRIFLTEQPLEGLEKLYQEPRELPPEERAQNVTRL
LRKQAEEVNTEWEKLNLHSADWQRKIDETLERLQELQEATDELDLKLRQAEVIKGSWQPVG
DLLIDSLQDHLEKVKALRGEIAPLKENVSHVNDLARQLTTLGIQLSPYNLSTLEDLNTRWKLL
QVAVEDRVRQLHEAHRDFGPASQHFLSTSVQGPWERAISPNKVPYYINHETQTTCWDHPKM
TELYQSLADLNNVRFSAYRTAMKLRRLQKALCLDLLSLSAACDALDQHNLKQNDQPMDILQ
IINCLTTIYDRLEQEHNNLVNVPLCVDMCLNWLLNVYDTGRTGRIRVLSFKTGIISLCKAHLE
DKYRYLFKQVASSTGFCDQRRLGLLLHDSIQIPRQLGEVASFGGSNIEPSVRSCFQFANNKPEI
EAALFLDWMRLEPQSMVWLPVLHRVAAAETAKHQAKCNICKECPIIGFRYRSLKHFNYDICQ
SCFFSGRVAKGHKMHYPMVEYCTPTTSGEDVRDFAKVLKNKFRTKRYFAKHPRMGYLPVQ
TVLEGDNMETPVTLINFWPVDSAPASSPQLSHDDTHSRIEHYASRLAEMENSNGSYLNDSISP
NESIDDEHLLIQHYCQSLNQDSPLSQPRSPAQILISLESEERGELERILADLEEENRNLQAEYDR
LKQQHEHKGLSPLPSPPEMMPTSPQSPRDAELIAEAKLLRQHKGRLEARMQILEDHNKQLES
QLHRLRQLLEQPQAEAKVNGTTVSSPSTSLQRSDSSQPMLLRVVGSQTSDSMGEEDLLSPPQ
DTSTGLEEVMEQLNNSFPSSRGRNTPGKPMREDTM,
dystrophin isoform Dp427m, coding sequence (CDS), NCBI reference
number CCDS14233.1, 11058 nucleotides (nt)
SEQ ID NO: 58
ATGCTTTGGTGGGAAGAAGTAGAGGACTGTTATGAAAGAGAAGATGTTCAAAAGAAAAC
ATTCACAAAATGGGTAAATGCACAATTTTCTAAGTTTGGGAAGCAGCATATTGAGAACCT
CTTCAGTGACCTACAGGATGGGAGGCGCCTCCTAGACCTCCTCGAAGGCCTGACAGGGC
AAAAACTGCCAAAAGAAAAAGGATCCACAAGAGTTCATGCCCTGAACAATGTCAACAAG
GCACTGCGGGTTTTGCAGAACAATAATGTTGATTTAGTGAATATTGGAAGTACTGACATC
GTAGATGGAAATCATAAACTGACTCTTGGTTTGATTTGGAATATAATCCTCCACTGGCAG
GTCAAAAATGTAATGAAAAATATCATGGCTGGATTGCAACAAACCAACAGTGAAAAGAT
TCTCCTGAGCTGGGTCCGACAATCAACTCGTAATTATCCACAGGTTAATGTAATCAACTT
CACCACCAGCTGGTCTGATGGCCTGGCTTTGAATGCTCTCATCCATAGTCATAGGCCAGA
CCTATTTGACTGGAATAGTGTGGTTTGCCAGCAGTCAGCCACACAACGACTGGAACATGC
ATTCAACATCGCCAGATATCAATTAGGCATAGAGAAACTACTCGATCCTGAAGATGTTGA
TACCACCTATCCAGATAAGAAGTCCATCTTAATGTACATCACATCACTCTTCCAAGTTTTG
CCTCAACAAGTGAGCATTGAAGCCATCCAGGAAGTGGAAATGTTGCCAAGGCCACCTAA
AGTGACTAAAGAAGAACATTTTCAGTTACATCATCAAATGCACTATTCTCAACAGATCAC
GGTCAGTCTAGCACAGGGATATGAGAGAACTTCTTCCCCTAAGCCTCGATTCAAGAGCTA
TGCCTACACACAGGCTGCTTATGTCACCACCTCTGACCCTACACGGAGCCCATTTCCTTC
ACAGCATTTGGAAGCTCCTGAAGACAAGTCATTTGGCAGTTCATTGATGGAGAGTGAAG
TAAACCTGGACCGTTATCAAACAGCTTTAGAAGAAGTATTATCGTGGCTTCTTTCTGCTG
AGGACACATTGCAAGCACAAGGAGAGATTTCTAATGATGTGGAAGTGGTGAAAGACCAG
TTTCATACTCATGAGGGGTACATGATGGATTTGACAGCCCATCAGGGCCGGGTTGGTAAT
ATTCTACAATTGGGAAGTAAGCTGATTGGAACAGGAAAATTATCAGAAGATGAAGAAAC
TGAAGTACAAGAGCAGATGAATCTCCTAAATTCAAGATGGGAATGCCTCAGGGTAGCTA
GCATGGAAAAACAAAGCAATTTACATAGAGTTTTAATGGATCTCCAGAATCAGAAACTG
AAAGAGTTGAATGACTGGCTAACAAAAACAGAAGAAAGAACAAGGAAAATGGAGGAAG
AGCCTCTTGGACCTGATCTTGAAGACCTAAAACGCCAAGTACAACAACATAAGGTGCTTC
AAGAAGATCTAGAACAAGAACAAGTCAGGGTCAATTCTCTCACTCACATGGTGGTGGTA
GTTGATGAATCTAGTGGAGATCACGCAACTGCTGCTTTGGAAGAACAACTTAAGGTATTG
GGAGATCGATGGGCAAACATCTGTAGATGGACAGAAGACCGCTGGGTTCTTTTACAAGA
CATCCTTCTCAAATGGCAACGTCTTACTGAAGAACAGTGCCTTTTTAGTGCATGGCTTTCA
GAAAAAGAAGATGCAGTGAACAAGATTCACACAACTGGCTTTAAAGATCAAAATGAAAT
GTTATCAAGTCTTCAAAAACTGGCCGTTTTAAAAGCGGATCTAGAAAAGAAAAAGCAAT
CCATGGGCAAACTGTATTCACTCAAACAAGATCTTCTTTCAACACTGAAGAATAAGTCAG
TGACCCAGAAGACGGAAGCATGGCTGGATAACTTTGCCCGGTGTTGGGATAATTTAGTCC
AAAAACTTGAAAAGAGTACAGCACAGATTTCACAGGCTGTCACCACCACTCAGCCATCA
CTAACACAGACAACTGTAATGGAAACAGTAACTACGGTGACCACAAGGGAACAGATCCT
GGTAAAGCATGCTCAAGAGGAACTTCCACCACCACCTCCCCAAAAGAAGAGGCAGATTA
CTGTGGATTCTGAAATTAGGAAAAGGTTGGATGTTGATATAACTGAACTTCACAGCTGGA
TTACTCGCTCAGAAGCTGTGTTGCAGAGTCCTGAATTTGCAATCTTTCGGAAGGAAGGCA
ACTTCTCAGACTTAAAAGAAAAAGTCAATGCCATAGAGCGAGAAAAAGCTGAGAAGTTC
AGAAAACTGCAAGATGCCAGCAGATCAGCTCAGGCCCTGGTGGAACAGATGGTGAATGA
GGGTGTTAATGCAGATAGCATCAAACAAGCCTCAGAACAACTGAACAGCCGGTGGATCG
AATTCTGCCAGTTGCTAAGTGAGAGACTTAACTGGCTGGAGTATCAGAACAACATCATCG
CTTTCTATAATCAGCTACAACAATTGGAGCAGATGACAACTACTGCTGAAAACTGGTTGA
AAATCCAACCCACCACCCCATCAGAGCCAACAGCAATTAAAAGTCAGTTAAAAATTTGT
AAGGATGAAGTCAACCGGCTATCAGATCTTCAACCTCAAATTGAACGATTAAAAATTCA
AAGCATAGCCCTGAAAGAGAAAGGACAAGGACCCATGTTCCTGGATGCAGACTTTGTGG
CCTTTACAAATCATTTTAAGCAAGTCTTTTCTGATGTGCAGGCCAGAGAGAAAGAGCTAC
AGACAATTTTTGACACTTTGCCACCAATGCGCTATCAGGAGACCATGAGTGCCATCAGGA
CATGGGTCCAGCAGTCAGAAACCAAACTCTCCATACCTCAACTTAGTGTCACCGACTATG
AAATCATGGAGCAGAGACTCGGGGAATTGCAGGCTTTACAAAGTTCTCTGCAAGAGCAA
CAAAGTGGCCTATACTATCTCAGCACCACTGTGAAAGAGATGTCGAAGAAAGCGCCCTC
TGAAATTAGCCGGAAATATCAATCAGAATTTGAAGAAATTGAGGGACGCTGGAAGAAGC
TCTCCTCCCAGCTGGTTGAGCATTGTCAAAAGCTAGAGGAGCAAATGAATAAACTCCGA
AAAATTCAGAATCACATACAAACCCTGAAGAAATGGATGGCTGAAGTTGATGTTTTTCTG
AAGGAGGAATGGCCTGCCCTTGGGGATTCAGAAATTCTAAAAAAGCAGCTGAAACAGTG
CAGACTTTTAGTCAGTGATATTCAGACAATTCAGCCCAGTCTAAACAGTGTCAATGAAGG
TGGGCAGAAGATAAAGAATGAAGCAGAGCCAGAGTTTGCTTCGAGACTTGAGACAGAAC
TCAAAGAACTTAACACTCAGTGGGATCACATGTGCCAACAGGTCTATGCCAGAAAGGAG
GCCTTGAAGGGAGGTTTGGAGAAAACTGTAAGCCTCCAGAAAGATCTATCAGAGATGCA
CGAATGGATGACACAAGCTGAAGAAGAGTATCTTGAGAGAGATTTTGAATATAAAACTC
CAGATGAATTACAGAAAGCAGTTGAAGAGATGAAGAGAGCTAAAGAAGAGGCCCAACA
AAAAGAAGCGAAAGTGAAACTCCTTACTGAGTCTGTAAATAGTGTCATAGCTCAAGCTC
CACCTGTAGCACAAGAGGCCTTAAAAAAGGAACTTGAAACTCTAACCACCAACTACCAG
TGGCTCTGCACTAGGCTGAATGGGAAATGCAAGACTTTGGAAGAAGTTTGGGCATGTTG
GCATGAGTTATTGTCATACTTGGAGAAAGCAAACAAGTGGCTAAATGAAGTAGAATTTA
AACTTAAAACCACTGAAAACATTCCTGGCGGAGCTGAGGAAATCTCTGAGGTGCTAGAT
TCACTTGAAAATTTGATGCGACATTCAGAGGATAACCCAAATCAGATTCGCATATTGGCA
CAGACCCTAACAGATGGCGGAGTCATGGATGAGCTAATCAATGAGGAACTTGAGACATT
TAATTCTCGTTGGAGGGAACTACATGAAGAGGCTGTAAGGAGGCAAAAGTTGCTTGAAC
AGAGCATCCAGTCTGCCCAGGAGACTGAAAAATCCTTACACTTAATCCAGGAGTCCCTCA
CATTCATTGACAAGCAGTTGGCAGCTTATATTGCAGACAAGGTGGACGCAGCTCAAATG
CCTCAGGAAGCCCAGAAAATCCAATCTGATTTGACAAGTCATGAGATCAGTTTAGAAGA
AATGAAGAAACATAATCAGGGGAAGGAGGCTGCCCAAAGAGTCCTGTCTCAGATTGATG
TTGCACAGAAAAAATTACAAGATGTCTCCATGAAGTTTCGATTATTCCAGAAACCAGCCA
ATTTTGAGCAGCGTCTACAAGAAAGTAAGATGATTTTAGATGAAGTGAAGATGCACTTG
CCTGCATTGGAAACAAAGAGTGTGGAACAGGAAGTAGTACAGTCACAGCTAAATCATTG
TGTGAACTTGTATAAAAGTCTGAGTGAAGTGAAGTCTGAAGTGGAAATGGTGATAAAGA
CTGGACGTCAGATTGTACAGAAAAAGCAGACGGAAAATCCCAAAGAACTTGATGAAAG
AGTAACAGCTTTGAAATTGCATTATAATGAGCTGGGAGCAAAGGTAACAGAAAGAAAGC
AACAGTTGGAGAAATGCTTGAAATTGTCCCGTAAGATGCGAAAGGAAATGAATGTCTTG
ACAGAATGGCTGGCAGCTACAGATATGGAATTGACAAAGAGATCAGCAGTTGAAGGAAT
GCCTAGTAATTTGGATTCTGAAGTTGCCTGGGGAAAGGCTACTCAAAAAGAGATTGAGA
AACAGAAGGTGCACCTGAAGAGTATCACAGAGGTAGGAGAGGCCTTGAAAACAGTTTTG
GGCAAGAAGGAGACGTTGGTGGAAGATAAACTCAGTCTTCTGAATAGTAACTGGATAGC
TGTCACCTCCCGAGCAGAAGAGTGGTTAAATCTTTTGTTGGAATACCAGAAACACATGGA
AACTTTTGACCAGAATGTGGACCACATCACAAAGTGGATCATTCAGGCTGACACACTTTT
GGATGAATCAGAGAAAAAGAAACCCCAGCAAAAAGAAGACGTGCTTAAGCGTTTAAAG
GCAGAACTGAATGACATACGCCCAAAGGTGGACTCTACACGTGACCAAGCAGCAAACTT
GATGGCAAACCGCGGTGACCACTGCAGGAAATTAGTAGAGCCCCAAATCTCAGAGCTCA
ACCATCGATTTGCAGCCATTTCACACAGAATTAAGACTGGAAAGGCCTCCATTCCTTTGA
AGGAATTGGAGCAGTTTAACTCAGATATACAAAAATTGCTTGAACCACTGGAGGCTGAA
ATTCAGCAGGGGGTGAATCTGAAAGAGGAAGACTTCAATAAAGATATGAATGAAGACA
ATGAGGGTACTGTAAAAGAATTGTTGCAAAGAGGAGACAACTTACAACAAAGAATCACA
GATGAGAGAAAGCGAGAGGAAATAAAGATAAAACAGCAGCTGTTACAGACAAAACATA
ATGCTCTCAAGGATTTGAGGTCTCAAAGAAGAAAAAAGGCTCTAGAAATTTCTCATCAGT
GGTATCAGTACAAGAGGCAGGCTGATGATCTCCTGAAATGCTTGGATGACATTGAAAAA
AAATTAGCCAGCCTACCTGAGCCCAGAGATGAAAGGAAAATAAAGGAAATTGATCGGG
AATTGCAGAAGAAGAAAGAGGAGCTGAATGCAGTGCGTAGGCAAGCTGAGGGCTTGTCT
GAGGATGGGGCCGCAATGGCAGTGGAGCCAACTCAGATCCAGCTCAGCAAGCGCTGGCG
GGAAATTGAGAGCAAATTTGCTCAGTTTCGAAGACTCAACTTTGCACAAATTCACACTGT
CCGTGAAGAAACGATGATGGTGATGACTGAAGACATGCCTTTGGAAATTTCTTATGTGCC
TTCTACTTATTTGACTGAAATCACTCATGTCTCACAAGCCCTATTAGAAGTGGAACAACT
TCTCAATGCTCCTGACCTCTGTGCTAAGGACTTTGAAGATCTCTTTAAGCAAGAGGAGTC
TCTGAAGAATATAAAAGATAGTCTACAACAAAGCTCAGGTCGGATTGACATTATTCATA
GCAAGAAGACAGCAGCATTGCAAAGTGCAACGCCTGTGGAAAGGGTGAAGCTACAGGA
AGCTCTCTCCCAGCTTGATTTCCAATGGGAAAAAGTTAACAAAATGTACAAGGACCGAC
AAGGGCGATTTGACAGATCTGTTGAGAAATGGCGGCGTTTTCATTATGATATAAAGATAT
TTAATCAGTGGCTAACAGAAGCTGAACAGTTTCTCAGAAAGACACAAATTCCTGAGAAT
TGGGAACATGCTAAATACAAATGGTATCTTAAGGAACTCCAGGATGGCATTGGGCAGCG
GCAAACTGTTGTCAGAACATTGAATGCAACTGGGGAAGAAATAATTCAGCAATCCTCAA
AAACAGATGCCAGTATTCTACAGGAAAAATTGGGAAGCCTGAATCTGCGGTGGCAGGAG
GTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGC
TAGTATCCCACTTGAACCTGGAAAAGAGCAGCAACTAAAAGAAAAGCTTGAGCAAGTCA
AGTTACTGGTGGAAGAGTTGCCCCTGCGCCAGGGAATTCTCAAACAATTAAATGAAACT
GGAGGACCCGTGCTTGTAAGTGCTCCCATAAGCCCAGAAGAGCAAGATAAACTTGAAAA
TAAGCTCAAGCAGACAAATCTCCAGTGGATAAAGGTTTCCAGAGCTTTACCTGAGAAAC
AAGGAGAAATTGAAGCTCAAATAAAAGACCTTGGGCAGCTTGAAAAAAAGCTTGAAGA
CCTTGAAGAGCAGTTAAATCATCTGCTGCTGTGGTTATCTCCTATTAGGAATCAGTTGGA
AATTTATAACCAACCAAACCAAGAAGGACCATTTGACGTTAAGGAAACTGAAATAGCAG
TTCAAGCTAAACAACCGGATGTGGAAGAGATTTTGTCTAAAGGGCAGCATTTGTACAAG
GAAAAACCAGCCACTCAGCCAGTGAAGAGGAAGTTAGAAGATCTGAGCTCTGAGTGGAA
GGCGGTAAACCGTTTACTTCAAGAGCTGAGGGCAAAGCAGCCTGACCTAGCTCCTGGAC
TGACCACTATTGGAGCCTCTCCTACTCAGACTGTTACTCTGGTGACACAACCTGTGGTTA
CTAAGGAAACTGCCATCTCCAAACTAGAAATGCCATCTTCCTTGATGTTGGAGGTACCTG
CTCTGGCAGATTTCAACCGGGCTTGGACAGAACTTACCGACTGGCTTTCTCTGCTTGATC
AAGTTATAAAATCACAGAGGGTGATGGTGGGTGACCTTGAGGATATCAACGAGATGATC
ATCAAGCAGAAGGCAACAATGCAGGATTTGGAACAGAGGCGTCCCCAGTTGGAAGAACT
CATTACCGCTGCCCAAAATTTGAAAAACAAGACCAGCAATCAAGAGGCTAGAACAATCA
TTACGGATCGAATTGAAAGAATTCAGAATCAGTGGGATGAAGTACAAGAACACCTTCAG
AACCGGAGGCAACAGTTGAATGAAATGTTAAAGGATTCAACACAATGGCTGGAAGCTAA
GGAAGAAGCTGAGCAGGTCTTAGGACAGGCCAGAGCCAAGCTTGAGTCATGGAAGGAG
GGTCCCTATACAGTAGATGCAATCCAAAAGAAAATCACAGAAACCAAGCAGTTGGCCAA
AGACCTCCGCCAGTGGCAGACAAATGTAGATGTGGCAAATGACTTGGCCCTGAAACTTC
TCCGGGATTATTCTGCAGATGATACCAGAAAAGTCCACATGATAACAGAGAATATCAAT
GCCTCTTGGAGAAGCATTCATAAAAGGGTGAGTGAGCGAGAGGCTGCTTTGGAAGAAAC
TCATAGATTACTGCAACAGTTCCCCCTGGACCTGGAAAAGTTTCTTGCCTGGCTTACAGA
AGCTGAAACAACTGCCAATGTCCTACAGGATGCTACCCGTAAGGAAAGGCTCCTAGAAG
ACTCCAAGGGAGTAAAAGAGCTGATGAAACAATGGCAAGACCTCCAAGGTGAAATTGA
AGCTCACACAGATGTTTATCACAACCTGGATGAAAACAGCCAAAAAATCCTGAGATCCC
TGGAAGGTTCCGATGATGCAGTCCTGTTACAAAGACGTTTGGATAACATGAACTTCAAGT
GGAGTGAACTTCGGAAAAAGTCTCTCAACATTAGGTCCCATTTGGAAGCCAGTTCTGACC
AGTGGAAGCGTCTGCACCTTTCTCTGCAGGAACTTCTGGTGTGGCTACAGCTGAAAGATG
ATGAATTAAGCCGGCAGGCACCTATTGGAGGCGACTTTCCAGCAGTTCAGAAGCAGAAC
GATGTACATAGGGCCTTCAAGAGGGAATTGAAAACTAAAGAACCTGTAATCATGAGTAC
TCTTGAGACTGTACGAATATTTCTGACAGAGCAGCCTTTGGAAGGACTAGAGAAACTCTA
CCAGGAGCCCAGAGAGCTGCCTCCTGAGGAGAGAGCCCAGAATGTCACTCGGCTTCTAC
GAAAGCAGGCTGAGGAGGTCAATACTGAGTGGGAAAAATTGAACCTGCACTCCGCTGAC
TGGCAGAGAAAAATAGATGAGACCCTTGAAAGACTCCGGGAACTTCAAGAGGCCACGG
ATGAGCTGGACCTCAAGCTGCGCCAAGCTGAGGTGATCAAGGGATCCTGGCAGCCCGTG
GGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACC
ACTTTGGGCATTCAGCTCTCACCGTATAACCTCAGCACTCTGGAAGACCTGAACACCAGA
TGGAAGCTTCTGCAGGTGGCCGTCGAGGACCGAGTCAGGCAGCTGCATGAAGCCCACAG
GGACTTTGGTCCAGCATCTCAGCACTTTCTTTCCACGTCTGTCCAGGGTCCCTGGGAGAG
AGCCATCTCGCCAAACAAAGTGCCCTACTATATCAACCACGAGACTCAAACAACTTGCTG
GGACCATCCCAAAATGACAGAGCTCTACCAGTCTTTAGCTGACCTGAATAATGTCAGATT
CTCAGCTTATAGGACTGCCATGAAACTCCGAAGACTGCAGAAGGCCCTTTGCTTGGATCT
CTTGAGCCTGTCAGCTGCATGTGATGCCTTGGACCAGCACAACCTCAAGCAAAATGACCA
GCCCATGGATATCCTGCAGATTATTAATTGTTTGACCACTATTTATGACCGCCTGGAGCA
AGAGCACAACAATTTGGTCAACGTCCCTCTCTGCGTGGATATGTGTCTGAACTGGCTGCT
GAATGTTTATGATACGGGACGAACAGGGAGGATCCGTGTCCTGTCTTTTAAAACTGGCAT
CATTTCCCTGTGTAAAGCACATTTGGAAGACAAGTACAGATACCTTTTCAAGCAAGTGGC
AAGTTCAACAGGATTTTGTGACCAGCGCAGGCTGGGCCTCCTTCTGCATGATTCTATCCA
AATTCCAAGACAGTTGGGTGAAGTTGCATCCTTTGGGGGCAGTAACATTGAGCCAAGTGT
CCGGAGCTGCTTCCAATTTGCTAATAATAAGCCAGAGATCGAAGCGGCCCTCTTCCTAGA
CTGGATGAGACTGGAACCCCAGTCCATGGTGTGGCTGCCCGTCCTGCACAGAGTGGCTGC
TGCAGAAACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGCTTTTTTT
CTGGTCGAGTTGCAAAAGGCCATAAAATGCACTATCCCATGGTGGAATATTGCACTCCGA
CTACATCAGGAGAAGATGTTCGAGACTTTGCCAAGGTACTAAAAAACAAATTTCGAACC
AAAAGGTATTTTGCGAAGCATCCCCGAATGGGCTACCTGCCAGTGCAGACTGTCTTAGAG
GGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGC
AGGCTAGCAGAAATGGAAAACAGCAATGGATCTTATCTAAATGATAGCATCTCTCCTAA
TGAGAGCATAGATGATGAACATTTGTTAATCCAGCATTACTGCCAAAGTTTGAACCAGGA
CTCCCCCCTGAGCCAGCCTCGTAGTCCTGCCCAGATCTTGATTTCCTTAGAGAGTGAGGA
AAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTC
CCCTCCTGAAATGATGCCCACCTCTCCCCAGAGTCCCCGGGATGCTGAGCTCATTGCTGA
GGCCAAGCTACTGCGTCAACACAAAGGCCGCCTGGAAGCCAGGATGCAAATCCTGGAAG
ACCACAATAAACAGCTGGAGTCACAGTTACACAGGCTAAGGCAGCTGCTGGAGCAACCC
CAGGCAGAGGCCAAAGTGAATGGCACAACGGTGTCCTCTCCTTCTACCTCTCTACAGAGG
TCCGACAGCAGTCAGCCTATGCTGCTCCGAGTGGTTGGCAGTCAAACTTCGGACTCCATG
GGTGAGGAAGATCTTCTCAGTCCTCCCCAGGACACAAGCACAGGGTTAGAGGAGGTGAT
GGAGCAACTCAACAACTCCTTCCCTAGTTCAAGAGGAAGAAATACCCCTGGAAAGCCAA
TGAGAGAGGACACAATGTAG,
dystrophin isoform Dp427m, polypeptide, NCBI reference number
NP_003997.2, 3685 amino acids (aa); Sequence Motif A (163-WSDGLALNAL-172, SEQ ID NO: 1)
and Sequence Motif B (181-FDWNSVVCQQSATQRLEHAF-200, SEQ ID NO: 2) are bolded and
double-underlined:
SEQ ID NO: 59
MLWWEEVEDCYEREDVQKKTFTKWVNAQFSKFGKQHIENLFSDLQDGRRLLDLLEGLTGQ
KLPKEKGSTRVHALNNVNKALRVLQNNNVDLVNIGSTDIVDGNHKLTLGLIWNIILHWQVK
NVMKNIMAGLQQTNSEKILLSWVRQSTRNYPQVNVINFTTSWSDGLALNALIHSHRPDLFD
WNSVVCQQSATQRLEHAFNIARYQLGIEKLLDPEDVDTTYPDKKSILMYITSLFQVLPQQVS
IEAIQEVEMLPRPPKVTKEEHFQLHHQMHYSQQITVSLAQGYERTSSPKPRFKSYAYTQAAY
VTTSDPTRSPFPSQHLEAPEDKSFGSSLMESEVNLDRYQTALEEVLSWLLSAEDTLQAQGEIS
NDVEVVKDQFHTHEGYMMDLTAHQGRVGNILQLGSKLIGTGKLSEDEETEVQEQMNLLNSR
WECLRVASMEKQSNLHRVLMDLQNQKLKELNDWLTKTEERTRKMEEEPLGPDLEDLKRQV
QQHKVLQEDLEQEQVRVNSLTHMVVVVDESSGDHATAALEEQLKVLGDRWANICRWTEDR
WVLLQDILLKWQRLTEEQCLFSAWLSEKEDAVNKIHTTGFKDQNEMLSSLQKLAVLKADLE
KKKQSMGKLYSLKQDLLSTLKNKSVTQKTEAWLDNFARCWDNLVQKLEKSTAQISQAVTT
TQPSLTQTTVMETVTTVTTREQILVKHAQEELPPPPPQKKRQITVDSEIRKRLDVDITELHSWI
TRSEAVLQSPEFAIFRKEGNFSDLKEKVNAIEREKAEKFRKLQDASRSAQALVEQMVNEGVN
ADSIKQASEQLNSRWIEFCQLLSERLNWLEYQNNIIAFYNQLQQLEQMTTTAENWLKIQPTTP
SEPTAIKSQLKICKDEVNRLSDLQPQIERLKIQSIALKEKGQGPMFLDADFVAFTNHFKQVFSD
VQAREKELQTIFDTLPPMRYQETMSAIRTWVQQSETKLSIPQLSVTDYEIMEQRLGELQALQS
SLQEQQSGLYYLSTTVKEMSKKAPSEISRKYQSEFEEIEGRWKKLSSQLVEHCQKLEEQMNK
LRKIQNHIQTLKKWMAEVDVFLKEEWPALGDSEILKKQLKQCRLLVSDIQTIQPSLNSVNEG
GQKIKNEAEPEFASRLETELKELNTQWDHMCQQVYARKEALKGGLEKTVSLQKDLSEMHE
WMTQAEEEYLERDFEYKTPDELQKAVEEMKRAKEEAQQKEAKVKLLTESVNSVIAQAPPVA
QEALKKELETLTTNYQWLCTRLNGKCKTLEEVWACWHELLSYLEKANKWLNEVEFKLKTT
ENIPGGAEEISEVLDSLENLMRHSEDNPNQIRILAQTLTDGGVMDELINEELETFNSRWRELHE
EAVRRQKLLEQSIQSAQETEKSLHLIQESLTFIDKQLAAYIADKVDAAQMPQEAQKIQSDLTS
HEISLEEMKKHNQGKEAAQRVLSQIDVAQKKLQDVSMKFRLFQKPANFEQRLQESKMILDE
VKMHLPALETKSVEQEVVQSQLNHCVNLYKSLSEVKSEVEMVIKTGRQIVQKKQTENPKEL
DERVTALKLHYNELGAKVTERKQQLEKCLKLSRKMRKEMNVLTEWLAATDMELTKRSAVE
GMPSNLDSEVAWGKATQKEIEKQKVHLKSITEVGEALKTVLGKKETLVEDKLSLLNSNWIA
VTSRAEEWLNLLLEYQKHMETFDQNVDHITKWIIQADTLLDESEKKKPQQKEDVLKRLKAE
LNDIRPKVDSTRDQAANLMANRGDHCRKLVEPQISELNHRFAAISHRIKTGKASIPLKELEQF
NSDIQKLLEPLEAEIQQGVNLKEEDFNKDMNEDNEGTVKELLQRGDNLQQRITDERKREEIKI
KQQLLQTKHNALKDLRSQRRKKALEISHQWYQYKRQADDLLKCLDDIEKKLASLPEPRDER
KIKEIDRELQKKKEELNAVRRQAEGLSEDGAAMAVEPTQIQLSKRWREIESKFAQFRRLNFA
QIHTVREETMMVMTEDMPLEISYVPSTYLTEITHVSQALLEVEQLLNAPDLCAKDFEDLFKQE
ESLKNIKDSLQQSSGRIDIIHSKKTAALQSATPVERVKLQEALSQLDFQWEKVNKMYKDRQG
RFDRSVEKWRRFHYDIKIFNQWLTEAEQFLRKTQIPENWEHAKYKWYLKELQDGIGQRQTV
VRTLNATGEEIIQQSSKTDASILQEKLGSLNLRWQEVCKQLSDRKKRLEEQKNILSEFQRDLN
EFVLWLEEADNIASIPLEPGKEQQLKEKLEQVKLLVEELPLRQGILKQLNETGGPVLVSAPISP
EEQDKLENKLKQTNLQWIKVSRALPEKQGEIEAQIKDLGQLEKKLEDLEEQLNHLLLWLSPIR
NQLEIYNQPNQEGPFDVKETEIAVQAKQPDVEEILSKGQHLYKEKPATQPVKRKLEDLSSEW
KAVNRLLQELRAKQPDLAPGLTTIGASPTQTVTLVTQPVVTKETAISKLEMPSSLMLEVPALA
DFNRAWTELTDWLSLLDQVIKSQRVMVGDLEDINEMIIKQKATMQDLEQRRPQLEELITAAQ
NLKNKTSNQEARTIITDRIERIQNQWDEVQEHLQNRRQQLNEMLKDSTQWLEAKEEAEQVL
GQARAKLESWKEGPYTVDAIQKKITETKQLAKDLRQWQTNVDVANDLALKLLRDYSADDT
RKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRL
DNMNFKWSELRKKSLNIRSHLEASSDQWKRLHLSLQELLVWLQLKDDELSRQAPIGGDFPA
VQKQNDVHRAFKRELKTKEPVIMSTLETVRIFLTEQPLEGLEKLYQEPRELPPEERAQNVTRL
LRKQAEEVNTEWEKLNLHSADWQRKIDETLERLRELQEATDELDLKLRQAEVIKGSWQPVG
DLLIDSLQDHLEKVKALRGEIAPLKENVSHVNDLARQLTTLGIQLSPYNLSTLEDLNTRWKLL
QVAVEDRVRQLHEAHRDFGPASQHELSTSVQGPWERAISPNKVPYYINHETQTTCWDHPKM
TELYQSLADLNNVRFSAYRTAMKLRRLQKALCLDLLSLSAACDALDQHNLKQNDQPMDILQ
IINCLTTIYDRLEQEHNNLVNVPLCVDMCLNWLLNVYDTGRTGRIRVLSFKTGIISLCKAHLE
DKYRYLFKQVASSTGFCDQRRLGLLLHDSIQIPRQLGEVASFGGSNIEPSVRSCFQFANNKPEI
EAALFLDWMRLEPQSMVWLPVLHRVAAAETAKHQAKCNICKECPIIGFRYRSLKHFNYDICQ
SCFFSGRVAKGHKMHYPMVEYCTPTTSGEDVRDFAKVLKNKFRTKRYFAKHPRMGYLPVQ
TVLEGDNMETPVTLINFWPVDSAPASSPQLSHDDTHSRIEHYASRLAEMENSNGSYLNDSISP
NESIDDEHLLIQHYCQSLNQDSPLSQPRSPAQILISLESEERGELERILADLEEENRNLQAEYDR
LKQQHEHKGLSPLPSPPEMMPTSPQSPRDAELIAEAKLLRQHKGRLEARMQILEDHNKQLES
QLHRLRQLLEQPQAEAKVNGTTVSSPSTSLQRSDSSQPMLLRVVGSQTSDSMGEEDLLSPPQ
DTSTGLEEVMEQLNNSFPSSRGRNTPGKPMREDTM,
(pAAV-CK8e-H3uDys_SpA, H3μDys)
SEQ ID NO: 60
      1 cagcagctgc gcgctcgctc gctcactgag gccgcccggg caaagcccgg gcgtcgggcg
    61 acctttggtc gcccggcctc agtgagcgag cgagcgcgca gagagggagt ggccaactcc
  121 atcactaggg gttccttgta gttaatgatt aacccgccat gctacttatc tacaaacgct
  181 agcatgctgc ccatgtaagg aggcaaggcc tggggacacc cgagatgcct ggttataatt
  241 aacccagaca tgtggctgcc cccccccccc caacacctgc tgcctctaaa aataaccctg
  301 catgccatgt tcccggcgaa gggccagctg tcccccgcca gctagactca gcacttagtt
  361 taggaaccag tgagcaagtc agcccttggg gcagcccata caaggccatg gggctgggca
  421 agctgcacgc ctgggtccgg ggtgggcacg gtgcccgggc aacgagctga aagctcatct
  481 gctctcaggg gcccctccct ggggacagcc cctcctggct agtcacaccc tgtaggctcc
  541 tctatataac ccaggggcac aggggctgcc ctcattctac caccacctcc acagcacaga
  601 cagacactca ggagccagcc agcgttaacc gtacgttttt tttatcgctg ccttgatata
  661 cactttccac catgctgtgg tgggaagagg tcgaagattg ctacgaaaga gaggacgtgc
  721 agaagaagac attcaccaag tgggtgaacg cccagttcag taaatttgga aagcagcaca
  781 tcgagaatct gttttcagac ctgcaggatg gccggagact gctggatctg ctggaagggc
  841 tgaccggaca gaagctgccc aaagagaagg gctctacacg cgtccatgca ctgaacaatg
  901 tgaacaaagc cctgcgagtc ctgcagaaca ataacgtgga cctggtcaat atcgggagca
  961 ctgacattgt ggatggaaac cacaagctga ccctgggcct gatctggaat atcattctgc
1021 attggcaggt gaaaaatgtc atgaagaaca tcatggccgg cctgcagcag accaacagcg
1081 agaaaattct gctgagctgg gtgagacagt ccacaaggaa ttacccccaa gtgaatgtca
1141 tcaacttcac cacatcttgg agtgacgggc tggctctgaa cgcactgatt cacagccata
1201 gacctgacct gttcgattgg aatagtgtgg tctgccagca gtcagccacc cagagactgg
1261 agcacgcctt taacatcgct aggtatcagc tgggcattga aaagctgctg gacccagagg
1321 acgtggatac tacctacccc gacaagaaat ccatcctgat gtatattaca tctctgttcc
1381 aggtcctgcc acagcaggtg agcatcgagg ccattcagga agtggagatg ctgcccaggc
1441 cccctaaagt caccaaggag gaacactttc agctgcacca tcagatgcat tacagccagc
1501 agatcacagt gtccctggca caggggtatg agcgaactag ctccccaaaa ccccggttca
1561 agagctacgc ctatactcag gccgcttacg tgacaacttc cgatcccacc agaagccctt
1621 tcccatccca gcacctggaa gctcctgagg ataagagctt tggatctagt ctgatggaat
1681 ccgaggtgaa cctggacagg tatcagactg ccctggagga agtcctgtct tggctgctga
1741 gtgcagaaga caccctgcag gcccagggcg aaatctccaa cgatgtggag gtggtcaagg
1801 accagtttca cacacatgag gggtacatga tggatctgac tgcacaccag ggccgcgtgg
1861 ggaatatcct gcagctgggc tctaaactga ttggaaccgg caagctgagt gaggacgagg
1921 aaacagaagt gcaggagcag atgaatctgc tgaactctcg ctgggaatgt ctgcgagtgg
1981 cctctatgga gaagcagagt aatctgcacc gggtcctgat ggatctgcag aatcagaaac
2041 tgaaggagct gaacgactgg ctgaccaaga cagaggaaag aaccaggaaa atggaggaag
2101 agcccctggg ccctgacctg gaagatctga agcgacaggt ccagcagcat aaagtgctgc
2161 aggaggatct ggaacaggag caggtgcggg tcaacagcct gacacacatg gtggtcgtgg
2221 tcgatgaatc aagcggggac catgccactg cagccctgga agagcagctg aaggtgctgg
2281 gagacagatg ggctaatatc tgtcggtgga ccgaggatag atgggtgctg ctgcaggaca
2341 ttctgctgaa atggcagagg ctgacagaag agcagtgcct gttctcagcc tggctgagcg
2401 aaaaagagga tgctgtgaac aagatccaca ccacagggtt taaggaccag aatgaaatgc
2461 tgtcctctct gcagaaactg gcagtgctga aggccgatct ggagaagaaa aagcagtcta
2521 tgggaaaact gtacagtctg aagcaggacc tgctgagcac cctgaaaaac aagagcgtga
2581 ctcagaagac cgaagcttgg ctggataatt tcgcaaggtg ctgggacaac ctggtgcaga
2641 aactggagaa gtcaacagct cagatcagcc aggctgcacc tgggctgact accattggag
2701 caagcccaac tcagaccgtg acactggtca ctcagcccgt ggtcacaaaa gaaactgcca
2761 tctccaagct ggagatgcct agttcactga tgctggaact ggagaggctg caggaactgc
2821 aggaggcaac cgacgaactg gatctgaagc tgcgccaggc cgaagtgatc aagggatcct
2881 ggcagcccgt cggcgacctg ctgattgatt ctctgcagga ccacctggag aaagtgaagg
2941 ccctgagagg agaaatcgct cctctgaagg agaatgtgag ccatgtcaac gatctggcta
3001 ggcagctgac aactctgggc attcagctgt ccccatataa tctgtctact ctggaggacc
3061 tgaacacccg gtggaaactg ctgcaggtgg ccgtcgaaga togcgtgcga cagctgcacg
3121 aggctcatag agacttcggc ccagcatctc agcactttct gtcaacaagc gtgcaggggc
3181 cctgggagag ggctatcagt ccaaataagg tgccctacta tattaaccac gaaacacaga
3241 ccacatgttg ggatcatcct aaaatgactg agctgtacca gtcactggcc gacctgaata
3301 acgtgaggtt tagcgcttat cgcaccgcaa tgaaactgag gcgcctgcag aaggccctgt
3361 gtctggatct gctgtccctg tctgccgctt gcgacgctct ggatcagcac aatctgaagc
3421 agaacgatca gccaatggac atcctgcaga tcattaactg cctgactacc atctacgacc
3481 gcctggaaca ggagcataat aacctggtga atgtccccct gtgtgtggat atgtgcctga
3541 attggctgct gaacgtctat gacaccggac gcacaggccg gatcagagtg ctgagtttca
3601 agacaggcat catttcactg tgtaaagcac acctggagga caagtacaga tacctgttca
3661 agcaggtggc ctccagcacc gggttttgg atcagcgacg gctgggactg ctgctgcacg
3721 actccatcca gattccccgc cagctggggg aagtggcttc cttoggggg tctaacatcg
3781 agcctagtgt gcgatcatgt ttccagtttg caaataacaa gccagaaatt gaggcagccc
3841 tgtttctgga ttggatgcgg ctggagcctc agagcatggt gtggctgcca gtcctgcaca
3901 gagtggctgc agccgaaacc gccaaacatc aggctaagtg caacatctgt aaggagtgcc
3961 ctatcattgg attcaggtac cgctccctga aacatttcaa ctacgacatc tgtcagagtt
4021 gcttcttttc aggccgggtg gctaaggggc acaagatgca ttaccccatg gtcgaatatt
4081 gcactcctac aacttctggc gaggacgtgc gcgatttcgc taaggtcctg aaaaataagt
4141 tccgaaccaa acggtacttt gcaaagcatc cacggatggg atacctgccc gtgcagacag
4201 tgctggaggg ggataatatg gaaacagaca caatgtaaga agtcttttcc acatggcaga
4261 tgactcgaga togatggcgc gccaccggtg gctagtaata aaagatcctt attttcattg
4321 gatctgtgtg ttggtttttt gtgtgggtac cgtttgtaga taagtagcat ggcgggttaa
4381 tcattaacta caaggaaccc ctagtgatgg agttggccac tccctctctg cgcgctcgct
4441 cgctcactga ggccgggcga ccaaaggtcg cccgacgccc gggctttgcc cgggcggcct
4501 cagtgagcga gcgagcgcgc cagctggcgt aatagcgaag aggcccgcac cgatcgccct
4561 tcccaacagt tgcgcagcct gaatggcgaa tggaattcca gacgattgag cgtcaaaatg
4621 taggtatttc catgagcgtt tttcctgttg caatggctgg cggtaatatt gttctggata
4681 ttaccagcaa ggccgatagt ttgagttctt ctactcaggc aagtgatgtt attactaatc
4741 aaagaagtat tgcgacaacg gttaatttgc gtgatggaca gactctttta ctcggtggcc
4801 tcactgatta taaaaacact tctcaggatt ctggcgtacc gttcctgtct aaaatccctt
4861 taatcggcct cctgtttagc tcccgctctg attctaacga ggaaagcacg ttatacgtgc
4921 tcgtcaaagc aaccatagta cgcgccctgt agcggcgcat taagcgcggc gggtgtggtg
4981 gttacgcgca gcgtgaccgc tacacttgcc agogccctag cgcccgctcc tttcgctttc
5041 ttcccttcct ttctcgccac gttcgccggc tttccccgtc aagctctaaa togggggctc
5101 cctttagggt tccgatttag tgctttacgg cacctcgacc ccaaaaaact tgattagggt
5161 gatggttcac gtagtgggcc atcgccctga tagacggttt ttcgcccttt gacgttggag
5221 tccacgttct ttaatagtgg actcttgttc caaactggaa caacactcaa ccctatctog
5281 gtctattctt ttgatttata agggattttg ccgatttcgg cctattggtt aaaaaatgag
5341 ctgatttaac aaaaatttaa cgcgaatttt aacaaaatat taacgtttac aatttaaata
5401 tttgcttata caatcttcct gtttttgggg cttttctgat tatcaaccgg ggtacatatg
5461 attgacatgc tagttttacg attaccgttc atcgattctc ttgtttgctc cagactctca
5521 ggcaatgacc tgatagcctt tgtagagacc tctcaaaaat agctaccctc tccggcatga
5581 atttatcagc tagaacggtt gaatatcata ttgatggtga tttgactgtc tccggccttt
5641 ctcacccgtt tgaatcttta cctacacatt actcaggcat tgcatttaaa atatatgagg
5701 gttctaaaaa tttttatcct tgcgttgaaa taaaggctto tcccgcaaaa gtattacagg
5761 gtcataatgt ttttggtaca accgatttag ctttatgctc tgaggcttta ttgcttaatt
5821 ttgctaattc tttgccttgc ctgtatgatt tattggatgt tggaattcct gatgcggtat
5881 tttctcctta cgcatctgtg cggtatttca caccgcatat ggtgcactct cagtacaatc
5941 tgctctgatg ccgcatagtt aagccagccc cgacacccgc caacacccgc tgacgcgccc
6001 tgacgggctt gtctgctccc ggcatccgct tacagacaag ctgtgaccgt ctccgggagc
6061 tgcatgtgtc agaggttttc accgtcatca ccgaaacgcg cgagacgaaa gggcctcgtg
6121 atacgcctat ttttataggt taatgtcatg ataataatgg tttcttagac gtcaggtggc
6181 acttttcggg gaaatgtgcg cggaacccct atttgtttat ttttctaaat acattcaaat
6241 atgtatccgc tcatgagaca ataaccctga taaatgcttc aataatattg aaaaaggaag
6301 agtatgagta ttcaacattt ccgtgtcgcc cttattccct tttttgcggc attttgcctt
6361 cctgtttttg ctcacccaga aacgctggtg aaagtaaaag atgctgaaga tcagttgggt
6421 gcacgagtgg gttacatcga actggatctc aacagcggta agatccttga gagttttcgc
6481 cccgaagaac gttttccaat gatgagcact tttaaagttc tgctatgtgg cgcggtatta
6541 tcccgtattg acgccgggca agagcaactc ggtcgccgca tacactattc tcagaatgac
6601 ttggttgagt actcaccagt cacagaaaag catcttacgg atggcatgac agtaagagaa
6661 ttatgcagtg ctgccataac catgagtgat aacactgcgg ccaacttact tctgacaacg
6721 atcggaggac cgaaggagct aaccgctttt ttgcacaaca tgggggatca tgtaactcgc
6781 cttgatogtt gggaaccgga gctgaatgaa gccataccaa acgacgagcg tgacaccacg
6841 atgcctgtag caatggcaac aacgttgcgc aaactattaa ctggcgaact acttactcta
6901 gcttcccggc aacaattaat agactggatg gaggcggata aagttgcagg accacttctg
6961 cgctcggccc ttccggctgg ctggtttatt gctgataaat ctggagccgg tgagcgtggg
7021 tctcgcggta tcattgcagc actggggcca gatggtaagc cctcccgtat cgtagttatc
7081 tacacgacgg ggagtcaggc aactatggat gaacgaaata gacagatcgc tgagataggt
7141 gcctcactga ttaagcattg gtaactgtca gaccaagttt actcatatat actttagatt
7201 gatttaaaac ttcattttta atttaaaagg atctaggtga agatcctttt tgataatctc
7261 atgaccaaaa tcccttaacg tgagttttcg ttccactgag cgtcagaccc cgtagaaaag
7321 atcaaaggat cttcttgaga tccttttttt ctgcgcgtaa tctgctgctt gcaaacaaaa
7381 aaaccaccgc taccagcggt ggtttgtttg ccggatcaag agctaccaac tctttttccg
7441 aaggtaactg gcttcagcag agcgcagata ccaaatactg tccttctagt gtagccgtag
7501 ttaggccacc acttcaagaa ctctgtagca ccgcctacat acctcgctct gctaatcctg
7561 ttaccagtgg ctgctgccag tggcgataag togtgtctta ccgggttgga ctcaagacga
7621 tagttaccgg ataaggcgca gcggtcgggc tgaacggggg gttcgtgcac acagcccagc
7681 ttggagcgaa cgacctacac cgaactgaga tacctacagc gtgagctatg agaaagcgcc
7741 acgcttcccg aagggagaaa ggcggacagg tatccggtaa gcggcagggt cggaacagga
7801 gagcgcacga gggagcttcc agggggaaac gcctggtatc tttatagtcc tgtcgggttt
7861 cgccacctct gacttgagcg tcgatttttg tgatgctcgt caggggggcg gagcctatgg
7921 aaaaacgcca gcaacgcggc ctttttacgg ttcctggcct tttgctggcc ttttgctcac
7981 atgttctttc ctgcgttatc ccctgattct gtggataacc gtattaccgc ctttgagtga
8041 gctgataccg ctcgccgcag ccgaacgacc gagcgcagcg agtcagtgag cgaggaagcg
8101 gaagagcgcc caatacgcaa accgcctctc cccgcgcgtt ggccgattca ttaatg
(Codon-Optimized H3μDys); Sequence Motif A (163-WSDGLALNAL-
172, SEQ ID NO: 1) and Sequence Motif B (181-FDWNSVVCQQSATQRLEHAF-200, SEQ ID NO:
2) are bolded and double-underlined:
SEQ ID NO: 61
MLWWEEVEDCYEREDVQKKTFTKWVNAQFSKFGKQHIENLFSDLQDGRRLLDLLEGLTGQ
KLPKEKGSTRVHALNNVNKALRVLQNNNVDLVNIGSTDIVDGNHKLTLGLIWNIILHWQVK
NVMKNIMAGLQQTNSEKILLSWVRQSTRNYPQVNVINFTTSWSDGLALNALIHSHRPDLFD
WNSVVCQQSATQRLEHAFNIARYQLGIEKLLDPEDVDTTYPDKKSILMYITSLFQVLPQQVS
IEAIQEVEMLPRPPKVTKEEHFQLHHQMHYSQQITVSLAQGYERTSSPKPRFKSYAYTQAAY
VTTSDPTRSPFPSQHLEAPEDKSFGSSLMESEVNLDRYQTALEEVLSWLLSAEDTLQAQGEIS
NDVEVVKDQFHTHEGYMMDLTAHQGRVGNILQLGSKLIGTGKLSEDEETEVQEQMNLLNSR
WECLRVASMEKQSNLHRVLMDLQNQKLKELNDWLTKTEERTRKMEEEPLGPDLEDLKRQV
QQHKVLQEDLEQEQVRVNSLTHMVVVVDESSGDHATAALEEQLKVLGDRWANICRWTEDR
WVLLQDILLKWQRLTEEQCLFSAWLSEKEDAVNKIHTTGFKDQNEMLSSLQKLAVLKADLE
KKKQSMGKLYSLKQDLLSTLKNKSVTQKTEAWLDNFARCWDNLVQKLEKSTAQISQAAPG
LTTIGASPTQTVTLVTQPVVTKETAISKLEMPSSLMLELERLQELQEATDELDLKLRQAEVIKG
SWQPVGDLLIDSLQDHLEKVKALRGEIAPLKENVSHVNDLARQLTTLGIQLSPYNLSTLEDLN
TRWKLLQVAVEDRVRQLHEAHRDFGPASQHELSTSVQGPWERAISPNKVPYYINHETQTTC
WDHPKMTELYQSLADLNNVRFSAYRTAMKLRRLQKALCLDLLSLSAACDALDQHNLKQND
QPMDILQIINCLTTIYDRLEQEHNNLVNVPLCVDMCLNWLLNVYDTGRTGRIRVLSFKTGIISL
CKAHLEDKYRYLFKQVASSTGFCDQRRLGLLLHDSIQIPRQLGEVASFGGSNIEPSVRSCFQF
ANNKPEIEAALFLDWMRLEPQSMVWLPVLHRVAAAETAKHQAKCNICKECPIIGFRYRSLKH
FNYDICQSCFFSGRVAKGHKMHYPMVEYCTPTTSGEDVRDFAKVLKNKFRTKRYFAKHPRM
GYLPVQTVLEGDNMETDTM
cysteine-rich (CR) domain
SEQ ID NO: 62
AHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQ
EHNNLVNVPLCVDMCLNWLLNVYDTGRTGRIRVLSFKTGIISLCKAHLEDKYRYLFKQVASS
TGFCDQRRLGLLLHDSIQIPRQLGEVASFGGSNIEPSVRSCFQFANNKPEIEAALFLDWMRLEP
QSMVWLPVLHRVAAAETAKHQAKCNICKECPIIGFRYRSLKHFNYDICQSCFFSGRVAKGHK
MHYPMVEYCTPTTSGEDVRDFAKVLKNKFRTKRYFAKHPRMGYLPVQTVLEGDNMET
(P17, NT-Des1)
SEQ ID NO: 63
ATGGCCGGCCTGCAGCAGACCAACAGCGAGAAAATTCTGCTGAGCTGGGTGAGACAGTC
CACAAGGAATTACCCCCAAGTGAATGTCATCAACTTCACCACATCTTGGAGTAATGGGCT
GGGACTGGACGCACTGATT
(P17, NT-Des2)
SEQ ID NO: 64
ATGGCCGGCCTGCAGCAGACCAACAGCGAGAAAATTCTGCTGAGCTGGGTGAGACAGTC
CACAAGGAATTACCCCCAAGTGAATGTCATCAACTTCACCACATCTTGGAGTGACGGGA
CAGGACTGAACGCACTGATT
(P17, NT-Des3)
SEQ ID NO: 65
ATGGCCGGCCTGCAGCAGACCAACAGCGAGAAAATTCTGCTGAGCTGGGTGAGACAGTC
CACAAGGAATTACCCCCAAGTGAATGTCATCAACTTCACCACATCTCTGAGTGACGGGAC
AGGACTGAACGCACTGATT
(P17, pAAV-CK8e-coh-H3uDys_SpA)
SEQ ID NO: 66
cagcagctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacaaacgctagcatgctgc
ccatgtaaggaggcaaggcctggggacacccgagatgcctggttataattaacccagacatgtggctgcccccccccccccaacacctgctgcct
ctaaaaataaccctgcatgccatgttcccggcgaagggccagctgtcccccgccagctagactcagcacttagtttaggaaccagtgagcaagtca
gcccttggggcagcccatacaaggccatggggctgggcaagctgcacgcctgggtccggggtgggcacggtgcccgggcaacgagctgaaa
gctcatctgctctcaggggcccctccctggggacagcccctcctggctagtcacaccctgtaggctcctctatataacccaggggcacaggggctg
ccctcattctaccaccacctccacagcacagacagacactcaggagccagccagcgttaaccgtacgttttttttatcgctgccttgatatacactttcc
accatgctgtggtgggaagaggtcgaagattgctacgaaagagaggacgtgcagaagaagacattcaccaagtgggtgaacgcccagttcagta
aatttggaaagcagcacatcgagaatctgttttcagacctgcaggatggccggagactgctggatctgctggaagggctgaccggacagaagctg
cccaaagagaagggctctacacgcgtccatgcactgaacaatgtgaacaaagccctgcgagtcctgcagaacaataacgtggacctggtcaatat
cgggagcactgacattgtggatggaaaccacaagctgaccctgggcctgatctggaatatcattctgcattggcaggtgaaaaatgtcatgaagaa
catcatggccggcctgcagcagaccaacagcgagaaaattctgctgagctgggtgagacagtccacaaggaattacccccaagtgaatgtcatca
acttcaccacatcttggagtgacgggctggctctgaacgcactgattcacagccatagacctgacctgttcgattggaatagtgtggtctgccagcag
tcagccacccagagactggagcacgcctttaacatcgctaggtatcagctgggcattgaaaagctgctggacccagaggacgtggatactacctac
cccgacaagaaatccatcctgatgtatattacatctctgttccaggtcctgccacagcaggtgagcatcgaggccattcaggaagtggagatgctgc
ccaggccccctaaagtcaccaaggaggaacactttcagctgcaccatcagatgcattacagccagcagatcacagtgtccctggcacaggggtat
gagcgaactagctccccaaaaccccggttcaagagctacgcctatactcaggccgcttacgtgacaacttccgatcccaccagaagccctttcccat
cccagcacctggaagctcctgaggataagagctttggatctagtctgatggaatccgaggtgaacctggacaggtatcagactgccctggaggaa
gtcctgtcttggctgctgagtgcagaagacaccctgcaggcccagggcgaaatctccaacgatgtggaggtggtcaaggaccagtttcacacaca
tgaggggtacatgatggatctgactgcacaccagggccgcgtggggaatatcctgcagctgggctctaaactgattggaaccggcaagctgagtg
aggacgaggaaacagaagtgcaggagcagatgaatctgctgaactctcgctgggaatgtctgcgagtggcctctatggagaagcagagtaatctg
caccgggtcctgatggatctgcagaatcagaaactgaaggagctgaacgactggctgaccaagacagaggaaagaaccaggaaaatggagga
agagcccctgggccctgacctggaagatctgaagcgacaggtccagcagcataaagtgctgcaggaggatctggaacaggagcaggtgcggg
tcaacagcctgacacacatggtggtcgtggtcgatgaatcaagcggggaccatgccactgcagccctggaagagcagctgaaggtgctgggag
acagatgggctaatatctgtcggtggaccgaggatagatgggtgctgctgcaggacattctgctgaaatggcagaggctgacagaagagcagtgc
ctgttctcagcctggctgagcgaaaaagaggatgctgtgaacaagatccacaccacagggtttaaggaccagaatgaaatgctgtcctctctgcag
aaactggcagtgctgaaggccgatctggagaagaaaaagcagtctatgggaaaactgtacagtctgaagcaggacctgctgagcaccctgaaaa
acaagagcgtgactcagaagaccgaagcttggctggataatttcgcaaggtgctgggacaacctggtgcagaaactggagaagtcaacagctca
gatcagccaggctgcacctgggctgactaccattggagcaagcccaactcagaccgtgacactggtcactcagcccgtggtcacaaaagaaactg
ccatctccaagctggagatgcctagttcactgatgctggaactggagaggctgcaggaactgcaggaggcaaccgacgaactggatctgaagct
gcgccaggccgaagtgatcaagggatcctggcagcccgtcggcgacctgctgattgattctctgcaggaccacctggagaaagtgaaggccctg
agaggagaaatcgctcctctgaaggagaatgtgagccatgtcaacgatctggctaggcagctgacaactctgggcattcagctgtccccatataatc
tgtctactctggaggacctgaacacccggtggaaactgctgcaggtggccgtcgaagatcgcgtgcgacagctgcacgaggctcatagagacttc
ggcccagcatctcagcactttctgtcaacaagcgtgcaggggccctgggagagggctatcagtccaaataaggtgccctactatattaaccacgaa
acacagaccacatgttgggatcatcctaaaatgactgagctgtaccagtcactggccgacctgaataacgtgaggtttagcgcttatcgcaccgcaa
tgaaactgaggcgcctgcagaaggccctgtgtctggatctgctgtccctgtctgccgcttgcgacgctctggatcagcacaatctgaagcagaacg
atcagccaatggacatcctgcagatcattaactgcctgactaccatctacgaccgcctggaacaggagcataataacctggtgaatgtccccctgtgt
gtggatatgtgcctgaattggctgctgaacgtctatgacaccggacgcacaggccggatcagagtgctgagtttcaagacaggcatcatttcactgt
gtaaagcacacctggaggacaagtacagatacctgttcaagcaggtggcctccagcaccgggttttgcgatcagcgacggctgggactgctgctg
cacgactccatccagattccccgccagctgggggaagtggcttccttcggcgggtctaacatcgagcctagtgtgcgatcatgtttccagtttgcaaa
taacaagccagaaattgaggcagccctgtttctggattggatgcggctggagcctcagagcatggtgtggctgccagtcctgcacagagtggctgc
agccgaaaccgccaaacatcaggctaagtgcaacatctgtaaggagtgccctatcattggattcaggtaccgctccctgaaacatttcaactacgac
atctgtcagagttgcttcttttcaggccgggtggctaaggggcacaagatgcattaccccatggtcgaatattgcactcctacaacttctggcgagga
cgtgcgcgatttcgctaaggtcctgaaaaataagttccgaaccaaacggtactttgcaaagcatccacggatgggatacctgcccgtgcagacagt
gctggagggggataatatggaaacagacacaatgtaagaagtcttttccacatggcagatgactcgagatcgatggcgcgccaccggtggctagt
aataaaagatccttattttcattggatctgtgtgttggttttttgtgtgggtaccgtttgtagataagtagcatgggggttaatcattaactacaaggaacc
cctagtgatggagttggccactccctctctgcgcgctcgctcgctcactgaggccgggcgaccaaaggtcgcccgacgcccgggctttgcccgg
gcggcctcagtgagcgagcgagcgcgccagctggcgtaatagcgaagaggcccgcaccgatcgcccttcccaacagttgcgcagcctgaatgg
cgaatggaattccagacgattgagcgtcaaaatgtaggtatttccatgagcgtttttcctgttgcaatggctggcggtaatattgttctggatattaccag
caaggccgatagtttgagttcttctactcaggcaagtgatgttattactaatcaaagaagtattgcgacaacggttaatttgcgtgatggacagactcttt
tactcggtggcctcactgattataaaaacacttctcaggattctggcgtaccgttcctgtctaaaatccctttaatcggcctcctgtttagctcccgctctg
attctaacgaggaaagcacgttatacgtgctcgtcaaagcaaccatagtacgcgccctgtagcggcgcattaagcgcggcgggtgtggtggttacg
cgcagcgtgaccgctacacttgccagcgccctagcgcccgctcctttcgctttcttcccttcctttctcgccacgttcgccggctttccccgtcaagctc
taaatcgggggctccctttagggttccgatttagtgctttacggcacctcgaccccaaaaaacttgattagggtgatggttcacgtagtgggccatogc
cctgatagacggtttttcgccctttgacgttggagtccacgttctttaatagtggactcttgttccaaactggaacaacactcaaccctatctcggtctatt
cttttgatttataagggattttgccgatttcggcctattggttaaaaaatgagctgatttaacaaaaatttaacgcgaattttaacaaaatattaacgtttaca
atttaaatatttgcttatacaatcttcctgtttttggggcttttctgattatcaaccggggtacatatgattgacatgctagttttacgattaccgttcatcga
ttctcttgtttgctccagactctcaggcaatgacctgatagcctttgtagagacctctcaaaaatagctaccctctccggcatgaatttatcagctagaacg
gttgaatatcatattgatggtgatttgactgtctccggcctttctcacccgtttgaatctttacctacacattactcaggcattgcatttaaaatatatgaggg
ttctaaaaatttttatccttgcgttgaaataaaggcttctcccgcaaaagtattacagggtcataatgtttttggtacaaccgatttagctttatgctctgagg
ctttattgcttaattttgctaattctttgccttgcctgtatgatttattggatgttggaattcctgatgcggtattttctccttacgcatctgtgcggtatttc
acaccgcatatggtgcactctcagtacaatctgctctgatgccgcatagttaagccagccccgacacccgccaacacccgctgacgcgccctgacgggct
tgtctgctcccggcatccgcttacagacaagctgtgaccgtctccgggagctgcatgtgtcagaggttttcaccgtcatcaccgaaacgcgcgagac
gaaagggcctcgtgatacgcctatttttataggttaatgtcatgataataatggtttcttagacgtcaggtggcacttttcggggaaatgtgcgcggaac
ccctatttgtttatttttctaaatacattcaaatatgtatccgctcatgagacaataaccctgataaatgcttcaataatattgaaaaaggaagagtatgagt
attcaacatttccgtgtcgcccttattcccttttttgcggcattttgccttcctgtttttgctcacccagaaacgctggtgaaagtaaaagatgctgaagatc
agttgggtgcacgagtgggttacatcgaactggatctcaacagcggtaagatccttgagagttttcgccccgaagaacgttttccaatgatgagcact
tttaaagttctgctatgtggcgcggtattatcccgtattgacgccgggcaagagcaactcggtcgccgcatacactattctcagaatgacttggttgag
tactcaccagtcacagaaaagcatcttacggatggcatgacagtaagagaattatgcagtgctgccataaccatgagtgataacactgcggccaact
tacttctgacaacgatcggaggaccgaaggagctaaccgcttttttgcacaacatgggggatcatgtaactcgccttgatcgttgggaaccggagct
gaatgaagccataccaaacgacgagcgtgacaccacgatgcctgtagcaatggcaacaacgttgcgcaaactattaactggcgaactacttactct
agcttcccggcaacaattaatagactggatggaggcggataaagttgcaggaccacttctgcgctcggcccttccggctggctggtttattgctgata
aatctggagccggtgagcgtgggtctcgcggtatcattgcagcactggggccagatggtaagccctcccgtatcgtagttatctacacgacgggga
gtcaggcaactatggatgaacgaaatagacagatcgctgagataggtgcctcactgattaagcattggtaactgtcagaccaagtttactcatatatac
tttagattgatttaaaacttcatttttaatttaaaaggatctaggtgaagatcctttttgataatctcatgaccaaaatcccttaacgtgagttttogttccac
tgagcgtcagaccccgtagaaaagatcaaaggatcttcttgagatcctttttttctgcgcgtaatctgctgcttgcaaacaaaaaaaccaccgctaccagc
ggtggtttgtttgccggatcaagagctaccaactctttttccgaaggtaactggcttcagcagagcgcagataccaaatactgtccttctagtgtagcc
gtagttaggccaccacttcaagaactctgtagcaccgcctacatacctcgctctgctaatcctgttaccagtggctgctgccagtggcgataagtcgt
gtcttaccgggttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacaggtatccggtaag
cggcagggtcggaacaggagagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttcctggccttttgctggccttttg
ctcacatgttctttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatg
In some embodiments of any of the aspects, a recombinant human dystrophin polypeptide
described herein comprises one or more mutations in the N-terminal actin-binding domain (e.g.,
amino acids ~1-245 (e.g., residues 1-245, 1-246, 9-244, 9-246, 14-240) of the dystrophin
polypeptide), see e.g., SEQ ID NOs: 67-68, in which Sequence Motif A (163-WSDGLALNAL-172,
SEQ ID NO: 1) and Sequence Motif B (181-FDWNSVVCQQSATQRLEHAF-200, SEQ ID NO: 2)
are bolded and double-underlined:
SEQ ID NO: 67
9_DSYEREDVQKKTFTKWVNAQFSKFGKQHIENLFSDLQDGRRLLDLLEGLTGQKLPKEKGS
TRVHALNNVNKALRVLQNNNVDLVNIGSTDIVDGNHKLTLGLIWNIILHWQVKNVMKNIMA
GLQQTNSEKILLSWVRQSTRNYPQVNVINFTTSWSDGLALNALIHSHRPDLFDWNSVVCQQ
SATQRLEHAFNIARYQLGIEKLLDPEDVDTTYPDKKSILMYITSLFQVLPQQVSIE_246
SEQ ID NO: 68
1-
MLWWEEVEDCYEREDVQKKTFTKWVNAQFSKFGKQHIENLFSDLQDGRRLLDLLEGLTGQ
KLPKEKGSTRVHALNNVNKALRVLQNNNVDLVNIGSTDIVDGNHKLTLGLIWNIILHWQVK
NVMKNIMAGLQQTNSEKILLSWVRQSTRNYPQVNVINFTTSWSDGLALNALIHSHRPDLFD
WNSVVCQQSATQRLEHAFNIARYQLGIEKLLDPEDVDTTYPDKKSILMYITSLFQVLPQQVS
IE -246
(P19 WT Ser)
SEQ ID NO: 69
FDWNSVVSQQSATQRLEHAF
(P19 WT Cys, with Cys residue bolded and double-underlined)
SEQ ID NO: 2
FDWNSVVCQQSATQRLEHAF
(P19 WT DNA from GENSCRIPT, previous codon optimization
(human/mouse), Cys codon bolded and double-underlined
SEQ ID NO: 70
TTCGATTGGAATAGTGTGGTCTGCCAGCAGTCAGCCACCCAGAGACTGGAGCACGCCTT
T
(P19_M1), Cys residue bolded and double-underlined, other mutations
bolded,
SEQ ID NO: 16
FDWNSVVCQKSAKYRLEHAF
(P19_M1), Cys codon bolded and double-underlined
SEQ ID NO: 71
TTCGATTGGAATAGTGTGGTCTGCCAGaagTCAGCCaagtacAGACTGGAGCACGCCTTT
(P19_M2), mutations bolded
SEQ ID NO: 17
FDWNSVVQQDSAKYRLQHAF
(P19_M2)
SEQ ID NO: 72
TTCGATTGGAATAGTGTGGTCcagCAGgacTCAGCCaagtacAGACTGcagCACGCCTTT
(P19_M3), mutations bolded
SEQ ID NO: 18
FDWNSVVKQDSAKYRLKHAF
(P19_M3)
SEQ ID NO: 73
TTCGATTGGAATAGTGTGGTCaagCAGgacTCAGCCaagtacAGACTGaagCACGCCTTT
(P19_M4), aspartic acid residue bolded and double-underlined, other
mutations bolded
SEQ ID NO: 19
FDWDSVVQQQSAKYRLQHAF
(P19_M4)
SEQ ID NO: 74
TTCGATTGGgacAGTGTGGTCcagCAGCAGTCAGCCaagtacAGACTGcagCACGCCTTT
(P19_M5), mutations bolded
SEQ ID NO: 20
FDWNSVVNQQSAQYRLKHAF
(P19_M5)
SEQ ID NO: 75
TTCGATTGGAATAGTGTGGTCaacCAGCAGTCAGCCcagtacAGACTGaagCACGCCTTT
(Wild type (MluI_Bsu36I) P19 epitope); MluI is bolded; Bsu36I is
italicized; P19 epitope is double-underlined:
SEQ ID NO: 76
ACGCGTCCATGCACTGAACAATGTGAACAAAGCCCTGCGAGTCCTGCAGAACAATAACG
TGGACCTGGTCAATATCGGGAGCACTGACATTGTGGATGGAAACCACAAGCTGACCCTG
GGCCTGATCTGGAATATCATTCTGCATTGGCAGGTGAAAAATGTCATGAAGAACATCATG
GCCGGCCTGCAGCAGACCAACAGCGAGAAAATTCTGCTGAGCTGGGTGAGACAGTCCAC
AAGGAATTACCCCCAAGTGAATGTCATCAACTTCACCACATCTTGGAGTGACGGGCTGGC
TCTGAACGCACTGATTCACAGCCATAGACCTGACCTGTTCGATTGGAATAGTGTGGTCTG
CCAGCAGTCAGCCACCCAGAGACTGGAGCACGCCTTTAACATCGCTAGGTATCAGCTGG
GCATTGAAAAGCTGCTGGACCCAGAGGACGTGGATACTACCTACCCCGACAAGAAATCC
ATCCTGATGTATATTACATCTCTGTTCCAGGTCCTGCCACAGCAGGTGAGCATCGAGGCC
ATTCAGGAAGTGGAGATGCTGCCCAGGCCCCCTAAAGTCACCAAGGAGGAACACTTTCA
GCTGCACCATCAGATGCATTACAGCCAGCAGATCACAGTGTCCCTGGCACAGGGGTATG
AGCGAACTAGCTCCCCAAAACCCCGGTTCAAGAGCTACGCCTATACTCAGGCCGCTTACG
TGACAACTTCCGATCCCACCAGAAGCCCTTTCCCATCCCAGCACCTGGAAGCTCCTGAGG
A
```

What is claimed is:

1. A recombinant human dystrophin polypeptide comprising one or more mutations in one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, wherein the recombinant polypeptide retains dystrophin activity.

2. The recombinant human dystrophin polypeptide of claim 1, wherein the polypeptide is a full-length dystrophin polypeptide, a mini-dystrophin polypeptide, or a microdystrophin polypeptide.

3. The recombinant human dystrophin polypeptide of claim 1, which comprises:
i) one or more mutations in Sequence Motif A that blunt T cell activity against a T cell epitope in Sequence Motif A;
ii) one or more mutations in Sequence Motif B that blunt T cell activity against a T cell epitope in Sequence Motif B; or
iii) one or more mutations in Sequence Motif A and Sequence Motif B that blunt T cell activity against T cell epitopes in Sequence Motifs A and B.

4-5. (canceled)

6. The recombinant human dystrophin polypeptide of claim 1, wherein the one or more mutations is/are substitution mutations.

7. The recombinant human dystrophin polypeptide of claim 1, wherein the mutation in Sequence Motif A comprises amino acid changes at positions 167 and 168.

8. (canceled)

9. The recombinant human dystrophin polypeptide of claim 7 wherein:
i) the mutation at position 167 is L167T,
ii) the mutation at position 168 is A168G, or
iii) the mutation at position 167 is L167T, and the mutation at position 168 is A168G.

10-11. (canceled)

12. The recombinant human dystrophin polypeptide of claim 1, wherein the mutation in Sequence Motif A comprises amino acid changes at positions 163, 167 and 168.

13. (canceled)

14. The recombinant human dystrophin polypeptide of claim 12, wherein:
i) the mutation at position 163 is W163L,
ii) the mutation at position 167 is L167T,
iii) the mutation at position 168 is A168G, or
iv) the mutation at position 163 is W163L, the mutation at position 167 is L167T, and the mutation at position 168 is A168G.

15-17. (canceled)

18. The recombinant human dystrophin polypeptide of claim 1, wherein;
i) the mutation in Sequence Motif B comprises amino acid changes at positions 188, 190, 193, 194 and 197; or
ii) the mutation in Sequence Motif B comprises amino acid changes at positions 184, 188, 190, 193, 194 and 197.

19-22. (canceled)

23. The recombinant human dystrophin polypeptide of claim 18, wherein:
i) the amino acid change at position 184 is N184D,
ii) the amino acid change at position 188 is C188Q, C188K or C188N,
iii) the amino acid change at position 190 is Q190D,
iv) the amino acid change at position 193 is T193K,
v) the amino acid change at position 194 is Q194Y, and/or
vi) the amino acid change at position 197 is E197Q or E197K.

24-27. (canceled)

28. A recombinant human dystrophin polypeptide in which:
i) the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9);
ii) the sequence motif 181-FDWNSVVCQQSATORLEHAF-200 (SEQ ID NO: 2) is mutated to 181-FDWNSVVQQDSAKYRLQHAF-200 (SEQ ID NO: 17), 181-FDWNSVVKQDSAKYRLKHAF-200 (SEQ ID NO: 18), or 181-FDWDSVVQQQSAKYRLOHAF-200 (SEQ ID NO: 19); or
iii) the sequence motif 163-WSDGLALNAL-172 (SEQ ID NO: 1) is mutated to 163-WSDGTGLNAL-172 (SEQ ID NO: 8) or 163-LSDGTGLNAL-172 (SEQ ID NO: 9), and the sequence motif 181-FDWNSVVCQQSATORLEHAF-200 (SEQ ID NO: 2) is mutated to 181-FDWNSVVQQDSAKYRLOHAF-200 (SEQ ID NO: 17), 181-FDWNSVVKQDSAKYRLKHAF-200 (SEQ ID NO: 18), or 181-FDWDSVVQQQSAKYRLOHAF-200 (SEQ ID NO: 19).

29-30. (canceled)

31. The recombinant dystrophin polypeptide of claim 28, wherein:
i) the dystrophin polypeptide retains dystrophin activity; and/or
ii) the dystrophin polypeptide exhibits blunted T cell activity against the polypeptide relative to wild-type dystrophin.

32. (canceled)

33. A method of reducing T cell activity against a recombinant human dystrophin polypeptide, the method comprising mutating one or both of Sequence Motif A (163-WSDGLALNAL-172, SEQ ID NO: 1) and Sequence Motif B (181-FDWNSVVCQQSATQRLEHAF-200, SEQ ID NO: 2) wherein the one or more mutations blunts T cell activity against the polypeptide relative to wild-type dystrophin, and wherein the recombinant polypeptide retains dystrophin activity.

34. (canceled)

35. A nucleic acid encoding a human recombinant dystrophin polypeptide of claim 1.

36. A vector comprising a nucleic acid of claim 35.

37-38. (canceled)

39. A pharmaceutical formulation comprising a nucleic acid of claim 35 and a pharmaceutically acceptable carrier.

40. A cell comprising a recombinant human dystrophin polypeptide of claim 1, or a nucleic acid or vector encoding such polypeptide.

41. A method of increasing dystrophin activity in a cell, the method comprising introducing a nucleic acid of claim 35 to a cell.

42. (canceled)

43. A method of treating a muscular dystrophy in a subject in need thereof, the method comprising introducing a nucleic acid of claim 35 to a muscle cell in the subject.

44. (canceled)

45. The method of claim 43, wherein the muscular dystrophy is myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, or an X-linked dilated cardiomyopathy.

46-47. (canceled)
